SEQUENCE LISTING (82 sequences)

SEQ ID NO: 1 - RNA, 42 nt, Streptococcus pyogenes
  misc_difference 1..20: note = n is a, c, g, t or u.
  source 1..42: mol_type = other RNA
nnnnnnnnnnnnnnnnnnnngttttagagctatgctgttttg

SEQ ID NO: 2 - RNA, 81 nt, Streptococcus pyogenes
  source 1..81: mol_type = other RNA
ggaaccattcaaaacagcatagcaagttaaaataaggctagtccgttatcaacttgaaaaagtggcaccgagtcggtgctt

SEQ ID NO: 3 - RNA, 94 nt, synthetic construct
  misc_feature 1..94: note = lgRNA, nn (32, 33) is either base-paired or not, and joined by non-nucleotide linker between the two, n32 (A) and n33 (B).
  misc_difference 1..20: note = n is a, c, g, t or u.
  misc_difference 32..33: note = n is a, c, g, t or u.
  source 1..94: mol_type = other RNA
nnnnnnnnnnnnnnnnnnnngttttagagctnnagcaagttaaaataaggctagtccgttatcaacttgaaaaagtggcaccgagtcggtgctt

SEQ ID NO: 4 - RNA, 100 nt, synthetic construct
  misc_feature 1..100: note = sgRNA: crRNA and tracrRNA are fused as a single whole RNA by a tetraloop, gaaa.
  misc_difference 1..20: note = n is a, c, g, t or u.
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 5 - RNA, 94 nt, synthetic construct; identical to SEQ ID NO: 3
  misc_feature 1..94: note = lgRNA formed by two ligations between nn (32, 33) of crgRNA and tracrgRNA, and between a70 and a71 in tracrgRNA.
  misc_difference 1..20: note = n is a, c, g, t or u.
  misc_difference 32..33: note = n is a, c, g, t or u.
  source 1..94: mol_type = other RNA

SEQ ID NO: 6 - RNA, 94 nt, synthetic construct
  misc_feature 1..94: note = gRNA inserted with an aptamer between a32 and u33. The aptamer sequence is not shown.
  misc_difference 1..20: note = n is a, c, g, t or u.
  source 1..94: mol_type = other RNA
nnnnnnnnnnnnnnnnnnnngttttagagctatagcaagttaaaataaggctagtccgttatcaacttgaaaaagtggcaccgagtcggtgctt

SEQ ID NO: 7 - RNA, 92 nt, synthetic construct
  misc_feature 1..92: note = gRNA inserted with two aptamers, positioned between a32 and u33 and between g69 and a70. The two aptamer sequences are not shown.
  misc_difference 1..20: note = n is a, c, g, t or u.
  source 1..92: mol_type = other RNA
nnnnnnnnnnnnnnnnnnnngttttagagctatagcaagttaaaataaggctagtccgttatcaacttgaaagtggcaccgagtcggtgctt

SEQ ID NO: 8 - RNA, 94 nt, synthetic construct; identical to SEQ ID NO: 3
  misc_feature 1..94: note = lgRNA inserted with two chemically ligated aptamers by nNt-linkers, positioned between n32 and n33 and between a70 and a71. The two aptamer sequences are not shown.
  misc_difference 1..20: note = n is a, c, g, t or u.
  misc_difference 32..33: note = n is a, c, g, t or u.
  source 1..94: mol_type = other RNA

SEQ ID NO: 9 - RNA, 62 nt, synthetic construct
  misc_feature 1..62: note = Chemically ligated tracrRNA inserted with two aptamers by nNt-linkers, positioned at 5'-end and n33 and between a38 and a39. The two aptamer sequences are not shown.
  misc_difference 1: note = n is a, c, g, t or u.
  source 1..62: mol_type = other RNA
nagcaagttaaaataaggctagtccgttatcaacttgaaaaagtggcaccgagtcggtgctt

SEQ ID NO: 10 - RNA, 95 nt, synthetic construct
  misc_feature 1..95: note = lgRNA conjugated with a chemically ligated aptamer at u33 (modified) and joined by an nNt-linker positioned between u33 and n34. The aptamer sequence is not shown.
  misc_difference 1..20: note = n is a, c, g, t or u.
  misc_difference 32: note = n is a, c, g, t or u.
  misc_difference 34: note = n is a, c, g, t or u.
  source 1..95: mol_type = other RNA
nnnnnnnnnnnnnnnnnnnngttttagagctntnagcaagttaaaataaggctagtccgttatcaacttgaaaaagtggcaccgagtcggtgctt

SEQ ID NO: 11 - RNA, 36 nt, Streptococcus pyogenes
  misc_difference 1..20: note = n is a, c, g, t or u.
  source 1..36: mol_type = other RNA
nnnnnnnnnnnnnnnnnnnngttttagagctatgct

SEQ ID NO: 12 - RNA, 66 nt, Streptococcus pyogenes
  source 1..66: mol_type = other RNA
agcatagcaagttaaaataaggctagtccgttatcaacttgaaaaagtggcaccgagtcggtgctt

SEQ ID NO: 13 - RNA, 47 nt, synthetic construct
  misc_feature 1..47: note = lgRNA (partial sequence) formed by chemical ligation bwteen tracrRNA and crRNA. The nNt-linker is positioned bwteen c15 (3'-end of tracrRNA) and g16 (5'-end of crRNA).
  misc_difference 28..47: note = n is a, c, g, t or u.
  source 1..47: mol_type = other RNA
ggaagggaaccctccggtgggttaaagnnnnnnnnnnnnnnnnnnnn

SEQ ID NO: 14 - RNA, 94 nt, synthetic construct; identical to SEQ ID NO: 3
  misc_feature 1..94: note = lgRNA, nn (32, 33) is either base-paired or not, and joined by non-nucleotide linker between the two, n32 (A) and n33 (B). GalAc ligands are conjugated at 3'-end of lgRNA via a Linker.
  misc_difference 1..20: note = n is a, c, g, t or u.
  misc_difference 32..33: note = n is a, c, g, t or u.
  source 1..94: mol_type = other RNA

SEQ ID NO: 15 - RNA, 47 nt, synthetic construct; identical to SEQ ID NO: 13
  misc_feature 1..47: note = lgRNA (partial sequence) formed by chemical ligation bwteen tracrRNA and crRNA. The nNt-linker of lgRNA is positioned bwteen c15 (3'-end of tracrRNA) and g16 (5'-end of crRNA). GalAc ligands are conjugated at 3'-end of lgRNA via a second Linker.
  misc_difference 28..47: note = n is a, c, g, t or u.
  source 1..47: mol_type = other RNA

SEQ ID NO: 16 - RNA, 42 nt, synthetic construct; identical to SEQ ID NO: 1
  misc_feature 1..42: note = Truncated crRNA with a general spacer.
  misc_difference 1..20: note = n is a, c, g, t or u.
  source 1..42: mol_type = other RNA

SEQ ID NO: 17 - RNA, 81 nt, synthetic construct; identical to SEQ ID NO: 2
  misc_feature 1..81: note = GalAc ligands are conjugated at 3'-end of tracrRNA via a Linker.
  source 1..81: mol_type = other RNA

SEQ ID NO: 18 - RNA, 42 nt, synthetic construct; identical to SEQ ID NO: 1
  misc_feature 1..42: note = A cholesterol is conjugated at 3'-end of crRNA via a Linker.
  misc_difference 1..20: note = n is a, c, g, t or u.
  source 1..42: mol_type = other RNA

SEQ ID NO: 19 - RNA, 42 nt, synthetic construct; identical to SEQ ID NO: 1
  misc_feature 1..42: note = Tocopherol is conjugated at 3'-end of crRNA via a Linker.
  misc_difference 1..20: note = n is a, c, g, t or u.
  source 1..42: mol_type = other RNA

SEQ ID NO: 20 - RNA, 42 nt, synthetic construct; identical to SEQ ID NO: 1
  misc_feature 1..42: note = An aptamer is conjugated at 3'-end of crRNA via a Linker. The aptamer sequence is not shown.
  misc_difference 1..20: note = n is a, c, g, t or u.
  source 1..42: mol_type = other RNA

SEQ ID NO: 21 - RNA, 42 nt, synthetic construct; identical to SEQ ID NO: 1
  misc_feature 1..42: note = An antibody is conjugated at 3'-end of crRNA via a Linker. The amino acid sequence of the antibody is not shown.
  misc_difference 1..20: note = n is a, c, g, t or u.
  source 1..42: mol_type = other RNA

SEQ ID NO: 22 - skipped

SEQ ID NO: 23 - RNA, 43 nt, synthetic construct
  misc_feature 1..43: note = crRNA-ssDNA conjugate with a linker between n43 and ssDNA. The sequence of ssDNA is not shown.
  misc_difference 21..43: note = n is a, c, g, t or u.
  source 1..43: mol_type = other RNA
taatttctactgttgtagatnnnnnnnnnnnnnnnnnnnnnnn

SEQ ID NO: 24 - RNA, 43 nt, synthetic construct
  misc_feature 1..43: note = crRNA-ssDNA conjugate with a linker between ssDNA and u1. The sequence of ssDNA is not shown.
  misc_difference 1..23: note = n is a, c, g, t or u.
  source 1..43: mol_type = other RNA
nnnnnnnnnnnnnnnnnnnnnnntaatttctactgttgtagat

SEQ ID NO: 25 - RNA, 43 nt, synthetic construct; identical to SEQ ID NO: 23
  misc_feature 1..43: note = ssDNA_crRNA-ssDNA conjugate with a linker between ssDNA and u1 and a second linker between n43 and a second ssDNA. The sequences of both ssDNA's are not shown.
  misc_difference 21..43: note = n is a, c, g, t or u.
  source 1..43: mol_type = other RNA

SEQ ID NO: 26 - DNA, 32 bp, synthetic construct
  misc_feature 1..32: note = crRNA-ssDNA conjugate with a linker between n32 and ssDNA. The sequence of ssDNA is not shown.
  misc_difference 1..20: note = n is a, c, g, t or u.
  misc_difference 32: note = n is a, c, g, t or u.
  source 1..32: mol_type = other DNA
nnnnnnnnnnnnnnnnnnnngttttagagctn

SEQ ID NO: 27 - DNA, 67 bp, synthetic construct
  misc_feature 1..67: note = tracrRNA-ssDNA conjugate with a linker between ssDNA and n1, The sequence of ssDNA is not shown.
  misc_difference 1: note = n is a, c, g, t or u.
  source 1..67: mol_type = other DNA
nagcatagcaagttaaaataaggctagtccgttatcaacttgaaaaagtggcaccgagtcggtgctt

SEQ ID NO: 28 - DNA, 94 bp, synthetic construct
  misc_feature 1..94: note = lgRNA formed by two ligations between nn (32, 33) of crRNA and tracrRNA, and between a70 and a71 in tracrRNA. An ssDNA is conjugated at the ligation site bwteen n32 and n33. The sequence of ssDNA is not shown.
  misc_difference 1..20: note = n is a, c, g, t or u.
  misc_difference 32..33: note = n is a, c, g, t or u.
  source 1..94: mol_type = other DNA
nnnnnnnnnnnnnnnnnnnngttttagagctnnagcaagttaaaataaggctagtccgttatcaacttgaaaaagtggcaccgagtcggtgctt

SEQ ID NO: 29 - DNA, 94 bp, synthetic construct; identical to SEQ ID NO: 28
  misc_feature 1..94: note = lgRNA formed by two ligations between nn (32, 33) of crRNA and tracrRNA, and between a70 and a71 in tracrRNA. An ssDNA is conjugated at the ligation site bwteen n70 and n71. The sequence of ssDNA is not shown.
  misc_difference 1..20: note = n is a, c, g, t or u.
  misc_difference 32..33: note = n is a, c, g, t or u.
  source 1..94: mol_type = other DNA

SEQ ID NO: 30 - DNA, 94 bp, synthetic construct; identical to SEQ ID NO: 28
  misc_feature 1..94: note = lgRNA formed by two ligations between nn (32, 33) of crRNA and tracrRNA, and between a70 and a71 in tracrRNA. An ssDNA is conjugated at the 3'-end (u94) of lgRNA via a nNt-Linker or nucleotide linker. The sequence of ssDNA is not shown.
  misc_difference 1..20: note = n is a, c, g, t or u.
  misc_difference 32..33: note = n is a, c, g, t or u.
  source 1..94: mol_type = other DNA

SEQ ID NO: 31 - DNA, 61 bp, synthetic construct
  misc_feature 1..61: note = tracrRNA formed by ligation between a37 and a38. An ssDNA is conjugated at the 3'-end (u61) of tracrRNA via a nNt-Linker or nucleotide linker. The sequence of ssDNA is not shown.
  source 1..61: mol_type = other DNA
agcaagttaaaataaggctagtccgttatcaacttgaaaaagtggcaccgagtcggtgctt

SEQ ID NO: 32 - DNA, 144 bp, synthetic construct
  misc_feature 1..144: note = lgRNA formed by two ligations between nn (82, 83) of crRNA and tracrRNA, and between a120 and a121 in tracrRNA. An ssDNA as an example is conjugated at the 5'-end of lgRNA via an nNt-Linker or nucleotide linker. The sequence of ssDNA is not shown.
  misc_difference 1..70: note = n is a, c, g, t or u.
  misc_difference 82..83: note = n is a, c, g, t or u.
  source 1..144: mol_type = other DNA
nnnnnnnnnnnnnnnnnnnnnnnnnnnnnnnnnnnnnnnnnnnnnnnnnnnnnnnnnnnnnnnnnnnnnngttttagagctnnagcaagttaaaataaggctagtccgttatcaacttgaaaaagtggcaccgagtcggtgctt

SEQ ID NO: 33 - DNA, 94 bp, synthetic construct; identical to SEQ ID NO: 28
  misc_feature 1..94: note = lgRNA formed by two ligations between nn (32, 33) of crRNA and tracrRNA, and between a70 and a71 in tracrRNA. An ssDNA is conjugated at the 5'-end of lgRNA via a Linker. A second ssDNA is conjugated at the 3'-end of lgRNA.
  misc_difference 1..20: note = n is a, c, g, t or u.
  misc_difference 32..33: note = n is a, c, g, t or u.
  source 1..94: mol_type = other DNA

SEQ ID NO: 34 - DNA, 95 bp, synthetic construct
  misc_feature 1..95: note = lgRNA formed by two ligations between n34 of crRNA and n35 of tracrRNA, and between a71 and a72 in tracrRNA. An ssDNA is conjugated between the 3'-end of ssDNA and n1.
  misc_difference 1..20: note = n is a, c, g, t or u.
  misc_difference 32: note = n is a, c, g, t or u.
  misc_difference 34: note = n is a,c, g, or u.
  source 1..95: mol_type = other DNA
nnnnnnnnnnnnnnnnnnnngttttagagctntnagcaagttaaaataaggctagtccgttatcaacttgaaaaagtggcaccgagtcggtgctt

SEQ ID NO: 35 - RNA, 95 nt, synthetic construct; identical to SEQ ID NO: 10
  misc_feature 1..95: note = lgRNA formed by two ligations between u33 of crRNA and n34 of tracrRNA, and between a71 and a72 in tracrRNA. An ssDNA is conjugated at the ligation site between nn (32, 33) via a Linker between the 3'-end of ssDNA and u33.
  misc_difference 1..20: note = n is a, c, g, t or u.
  misc_difference 32: note = n is a, c, g, t or u.
  misc_difference 34: note = n is a, c, g, t or u.
  source 1..95: mol_type = other RNA

SEQ ID NO: 36 - DNA, 144 bp, synthetic construct
  misc_feature 1..144: note = lgRNA formed by two ligations between nn (32, 33) of crRNA and tracrRNA, and between a70 and a71 in tracrRNA. An ssDNA is conjugated at the 3'-end of lgRNA via a triazole nNt-Linker between the 3'-end of lgRNA and the 5'-end of ssDNA.
  misc_difference 1..20: note = n is a, c, g, t or u.
  misc_difference 32..33: note = n is a, c, g, t or u.
  misc_difference 95..144: note = n is a, c, g, t or u.
  source 1..144: mol_type = other DNA
nnnnnnnnnnnnnnnnnnnngttttagagctnnagcaagttaaaataaggctagtccgttatcaacttgaaaaagtggcaccgagtcggtgcttnnnnnnnnnnnnnnnnnnnnnnnnnnnnnnnnnnnnnnnnnnnnnnnnnn

SEQ ID NO: 37 - DNA, 95 bp, synthetic construct; identical to SEQ ID NO: 34
  misc_feature 1..95: note = lgRNA formed by two ligations between u33 of crRNA and n34 of tracrRNA, and between a71 and a72 in tracrRNA. An ssDNA is conjugated at the ligation site between nn (32, 33) via an nNt-Linker between the 5'-end of ssDNA and u33.
  misc_difference 1..20: note = n is a, c, g, t or u.
  misc_difference 32: note = n is a, c, g, t or u.
  misc_difference 34: note = n is a, c, g, t or u.
  source 1..95: mol_type = other DNA

SEQ ID NO: 38 - RNA, 43 nt, synthetic construct; identical to SEQ ID NO: 23
  misc_feature 1..43: note = crRNA-ssDNA conjugate with an nNt-linker between n43 and the 5'-end of ssDNA .
  misc_difference 21..43: note = n is a, c, g, t or u.
  source 1..43: mol_type = other RNA

SEQ ID NO: 39 - DNA, 43 bp, synthetic construct
  misc_feature 1..43: note = crRNA-ssDNA conjugate with an nNt-linker between u1 and the 5'-end of ssDNA.
  misc_difference 21..43: note = n is a, c, g, t or u.
  source 1..43: mol_type = other DNA
taatttctactgttgtagatnnnnnnnnnnnnnnnnnnnnnnn

SEQ ID NO: 40 - DNA, 43 bp, synthetic construct; identical to SEQ ID NO: 39
  misc_feature 1..43: note = crRNA-ssDNA conjugate with an nNt-linker between n43 and the 3'-end of ssDNA.
  misc_difference 21..43: note = n is a, c, g, t or u.
  source 1..43: mol_type = other DNA

SEQ ID NO: 41 - DNA, 43 bp, synthetic construct; identical to SEQ ID NO: 39
  misc_feature 1..43: note = crRNA-ssDNA conjugate with an nNt-linker between u1 and the 3'-end of ssDNA.
  misc_difference 21..43: note = n is a, c, g, t or u.
  source 1..43: mol_type = other DNA

SEQ ID NO: 42 - DNA, 43 bp, synthetic construct; identical to SEQ ID NO: 39
  misc_feature 1..43: note = ssDNA-crRNA-ssDNA conjugate with an nNt-linker between u1 and the 3'-end of ssDNA, and a second nNt-linker between n43 and the 5'-end of a second ssDNA.
  misc_difference 21..43: note = n is a, c, g, t or u.
  source 1..43: mol_type = other DNA

SEQ ID NO: 43 - DNA, 194 bp, synthetic construct
  misc_feature 1..194: note = An ssDNA (n145 to n194) is conjugated at the 3'-end of lgRNA via a triazole nNt-Linker between the 3'-end of lgRNA and the 5'-end of ssDNA, and a second ssDNA s conjugated at the 5'-end of lgRNA.
  misc_difference 1..70: note = n is a, c, g, t or u.
  misc_difference 82..83: note = n is a, c, g, t or u.
  misc_difference 145..194: note = n is a, c, g, t or u.
  source 1..194: mol_type = other DNA
nnnnnnnnnnnnnnnnnnnnnnnnnnnnnnnnnnnnnnnnnnnnnnnnnnnnnnnnnnnnnnnnnnnnnngttttagagctnnagcaagttaaaataaggctagtccgttatcaacttgaaaaagtggcaccgagtcggtgcttnnnnnnnnnnnnnnnnnnnnnnnnnnnnnnnnnnnnnnnnnnnnnnnnnn

SEQ ID NO: 44 - DNA, 144 bp, synthetic construct; identical to SEQ ID NO: 36
  misc_feature 1..144: note = An ssDNA is conjugated at the 3'-end of lgRNA via a triazole nNt-Linker between the 3'-end of lgRNA and a dsDNA. Its complementary strand is not shown.
  misc_difference 1..20: note = n is a, c, g, t or u.
  misc_difference 32..33: note = n is a, c, g, t or u.
  misc_difference 95..144: note = n is a, c, g, t or u.
  source 1..144: mol_type = other DNA

SEQ ID NO: 45 - DNA, 23 bp, Hepatitis B virus
  source 1..23: mol_type = genomic DNA
ctctgctagatcccagagtgagg

SEQ ID NO: 46 - DNA, 23 bp, Hepatitis B virus
  source 1..23: mol_type = genomic DNA
gctatcgctggatgtgtctgcgg

SEQ ID NO: 47 - DNA, 25 bp, Hepatitis B virus
  source 1..25: mol_type = genomic DNA
tggacttctctcaattttctagggg

SEQ ID NO: 48 - DNA, 23 bp, Hepatitis B virus
  source 1..23: mol_type = genomic DNA
gggggatcacccgtgtgtcttgg

SEQ ID NO: 49 - DNA, 25 bp, Hepatitis B virus
  source 1..25: mol_type = genomic DNA
tatgtggatgatgtggtactggggg

SEQ ID NO: 50 - DNA, 21 bp, Hepatitis B virus
  source 1..21: mol_type = genomic DNA
cctcaccatacagcactcggg

SEQ ID NO: 51 - DNA, 26 bp, Hepatitis B virus
  source 1..26: mol_type = genomic DNA
gtgttggggtgagttgatgaatctgg

SEQ ID NO: 52 - DNA, 25 bp, Hepatitis B virus
  source 1..25: mol_type = genomic DNA
tatgtggatgatgtggtactggggg

SEQ ID NO: 53 - DNA, 25 bp, Hepatitis B virus
  source 1..25: mol_type = genomic DNA
tatatggatgatgtggtattggggg

SEQ ID NO: 54 - DNA, 25 bp, Hepatitis B virus
  source 1..25: mol_type = genomic DNA
tatgtggatgatgtggtattggggg

SEQ ID NO: 55 - DNA, 25 bp, Hepatitis B virus
  source 1..25: mol_type = genomic DNA
tatatagatgatgtggtactggggg

SEQ ID NO: 56 - DNA, 23 bp, Human immunodeficiency virus
  source 1..23: mol_type = genomic DNA
gattggcagaactacacaccagg

SEQ ID NO: 57 - DNA, 23 bp, Human immunodeficiency virus
  source 1..23: mol_type = genomic DNA
atcagatatccactgacctttgg

SEQ ID NO: 58 - DNA, 23 bp, Human immunodeficiency virus
  source 1..23: mol_type = genomic DNA
gcgtggcctgggcgggactgggg

SEQ ID NO: 59 - DNA, 23 bp, Human immunodeficiency virus
  source 1..23: mol_type = genomic DNA
cagcagttcttgaagtactccgg

SEQ ID NO: 60 - DNA, 23 bp, human herpesvirus
  source 1..23: mol_type = genomic DNA
gccctggaccaacccggcccggg

SEQ ID NO: 61 - DNA, 23 bp, human herpesvirus
  source 1..23: mol_type = genomic DNA
ggccgctgccccgctccgggtgg

SEQ ID NO: 62 - DNA, 22 bp, human herpesvirus
  source 1..22: mol_type = genomic DNA
ggaagacaatgtgccgccatgg

SEQ ID NO: 63 - DNA, 23 bp, human herpesvirus
  source 1..23: mol_type = genomic DNA
tctggaccagaaggctccggcgg

SEQ ID NO: 64 - DNA, 23 bp, human herpesvirus
  source 1..23: mol_type = genomic DNA
gctgccgcggagggtgatgacgg

SEQ ID NO: 65 - DNA, 23 bp, human herpesvirus
  source 1..23: mol_type = genomic DNA
ggtggcccaccgggtccgctggg

SEQ ID NO: 66 - DNA, 23 bp, human herpesvirus
  source 1..23: mol_type = genomic DNA
gtcctcgagggggccgtcgcggg

SEQ ID NO: 67 - DNA, 21 bp, human
  source 1..21: mol_type = genomic DNA
aaagaaaatatmmmtggtgtt

SEQ ID NO: 68 - DNA, 21 bp, human
  source 1..21: mol_type = genomic DNA
aaagaaaatatctttggtgtt

SEQ ID NO: 69 - RNA, 50 nt, synthetic construct
  misc_feature 1..50: note = GalAcs are conjugated at 3'-end of crRNA via an triazole nNt-Linker.
  source 1..50: mol_type = other RNA
gcgtggcctgggcgggactggttttagtactctgtaattttaggtatgag

SEQ ID NO: 70 - RNA, 31 nt, synthetic construct
  misc_feature 1..31: note = crRNA 3'-azide
  source 1..31: mol_type = other RNA
ctatatggatgatgtggtacgttttagagct

SEQ ID NO: 71 - RNA, 61 nt, synthetic construct
  misc_feature 1..61: note = 5'-alkynyl tracrRNA 3'-maleimide.
  source 1..61: mol_type = other RNA
agcaagttaaaataaggctagtccgttatcaacttgaaaaagtggcaccgagtcggtgctt

SEQ ID NO: 72 - RNA, 92 nt, synthetic construct
  misc_feature 1..92: note = lgRNA 3'-maleimide. lgRNA contains a triazole nNt-Linker between u31 and a32.
  source 1..92: mol_type = other RNA
ctatatggatgatgtggtacgttttagagctagcaagttaaaataaggctagtccgttatcaacttgaaaaagtggcaccgagtcggtgctt

SEQ ID NO: 73 - DNA, 37 bp, synthetic construct
  misc_feature 1..37: note = ssDNA, 5'-thiol
  source 1..37: mol_type = other DNA
tcagacttggcccccatcaatcaatcaatccatatag

SEQ ID NO: 74 - DNA, 129 bp, synthetic construct
  misc_feature 1..129: note = lgRNA-ssDNA conjugate with a thio-ether nNt-Linker (u93 and u94).
  source 1..129: mol_type = other DNA
ctatatggatgatgtggtacgttttagagctagcaagttaaaataaggctagtccgttatcaacttgaaaaagtggcaccgagtcggtgctttcagacttggcccccatcaatcaatcaatccatatag

SEQ ID NO: 75 - RNA, 101 nt, synthetic construct
  misc_feature 1..101: note = sgRNA 3'-maleimide.
  source 1..101: mol_type = other RNA
gctatatggatgatgtggtacgttttagagctagaaatagcaagttaaaataaggctagtccgttatcaacttgaaaaagtggcaccgagtcggtgctttt

SEQ ID NO: 76 - DNA, 138 bp, synthetic construct
  misc_feature 1..138: note = lgRNA-ssDNA conjugate with a thio-ether nNt-Linker (u101 and u102).
  source 1..138: mol_type = other DNA
gctatatggatgatgtggtacgttttagagctagaaatagcaagttaaaataaggctagtccgttatcaacttgaaaaagtggcaccgagtcggtgctttttcagacttggcccccatcaatcaatcaatccatatag

SEQ ID NO: 77 - DNA, 129 bp, synthetic construct
  misc_feature 1..129: note = ssDNA-lgRNA conjugate with a thio-ether nNt-Linker (g36 and u37). A second nNt-Linker is between u68 and a69.
  source 1..129: mol_type = other DNA
cagacttggcccccatcaatcaatcaatccatatagtctatatggatgatgtggtacgttttagagctagcaagttaaaataaggctagtccgttatcaacttgaaaaagtggcaccgagtcggtgctt

SEQ ID NO: 78 - DNA, 128 bp, synthetic construct
  misc_feature 1..128: note = lgRNA-ssDNA conjugate with an nNt-Linker between u31 and a32, and a second nNt-Linker (thio-ether ) between g67 and u68. c93-g129 is a DNA oligonucleotide.
  source 1..128: mol_type = other DNA
ctatatggatgatgtggtacgttttagagctagcaagttaaaataaggctagtccgttatcaacttgtaaagtggcaccgagtcggtgctttcagacttggcccccatcaatcaatcaatccatatag

SEQ ID NO: 79 - DNA, 84 bp, synthetic construct
  misc_feature 1..84: note = cDNA encoding SaCas9 tracrRNA.
  source 1..84: mol_type = other DNA
caccacttatacctaaaattacagaatctactaaaacaaggcaaaatgccgtgtttatctcgtcaacttgttggcgagattttt

SEQ ID NO: 80 - DNA, 84 bp, synthetic construct
  misc_feature 1..84: note = cDNA encoding SaCas9 tracrRNA.
  source 1..84: mol_type = other DNA
ggccaaaaatctcgccaacaagttgacgagataaacacggcattttgccttgttttagtagattctgtaattttaggtataagt

SEQ ID NO: 81 - DNA, 4104 bp, synthetic construct
  misc_feature 1..4104: note = DNA sequence of engineered SpCas9 protein by site-directed mutagenesis.
  source 1..4104: mol_type = other DNA
tgcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatcgcctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggactgcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgcctgcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagtgcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagtgcgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 82 - protein, 1392 aa, synthetic construct
  REGION 1..1392: note = Sequence of an engineered SpCas9-NLS protein.
CDKKYSIGLDIGTNSVGWAVITDEYKVPSKKFKVLGNTDRHSIKKNLIGALLFDSGETAEATRLKRTARRRYTRRKNRIAYLQEIFSNEMAKVDDSFFHRLEESFLVEEDKKHERHPIFGNIVDEVAYHEKYPTIYHLRKKLVCSTDKADLRLIYLALAHMIKFRGHFLIEGDLNPDNSDVDKLFIQLVQTYNQLFEENPINACGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKCEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKCVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVD

(19) United States
(12) Patent Application Publication
(10) Pub. No.: US 2025/0027115 A1
(43) Pub. Date: Jan. 23, 2025

(54) Conjugates of guide RNA-Cas protein complex for genome and epigenome editing

(71) Applicant: Minghong Zhong, Sellersville, PA (US)

(72) Inventor: Minghong ZHONG, Sellersville, PA (US)

(21) Appl. No.: 18/628,766

(22) Filed: Apr. 7, 2024

Related U.S. Application Data
(63) Continuation-in-part of application No. 16/994,660, filed on Aug. 17, 2020.
Application No. 16/994,660 is a continuation-in-part of application No. PCT/US2020/036860, filed on Jun. 10, 2020.
(60) Provisional application No. 62/888,551, filed on Aug. 19, 2019.
(60) Provisional application No. 62/914,565, filed on Oct. 14, 2019.
(60) Provisional application No. 62/937,876, filed on Nov. 20, 2019.

(30) Foreign Application Priority Data
Jun. 10, 2020 (CN) 202080073024.3

(51) Int. Cl. C12N 15/90; C12N 9/22; C12N 9/96; C12N 15/11

(52) U.S. Cl. CPC C12N 15/902; C12N 9/22; C12N 9/96; C12N 15/111; C12N 2310/20; C12N 2310/3341; C12N 2310/3513

(57) ABSTRACT

Provided herein are compositions of conjugates of a guide RNA(s)-CRISPR Cas protein (RNP) complex. The conjugate comprises a guide RNA(s)-CRISPR Cas protein (RNP) complex and one or more molecules selected from PEG, non-PEG polymers, ligands for cellular receptors, lipids, oligonucleotides, polysaccharides and peptides and chemically linked to the Cas protein and/or guide RNA(s). Also provided a guide RNA-DNA template conjugate comprises a DNA template with 5-methylcytosines at specific positions for epigenome editing by DNA methylation at a single nucleotide resolution level. The conjugates are delivered to targeted cells as RNP complexes, or formed in targeted cells from guide RNA conjugates and an mRNA or a viral vector encoding a Cas protein, or formed in targeted cells from a crRNA conjugate(s) and a viral vector encoding both a Cas protein and a tracrRNA. Also provided are preparation methods and uses of these conjugates.

### CROSS REFERENCE TO RELATED APPLICATIONS

The present application is a continuation in part of U.S. Nonprovisional application Ser. No. 16/994,660, filed on Aug. 17, 2020, which claims U.S. Provisional Applications Ser. No. 62/888,551, filed on Aug. 19, 2019, Ser. No. 62/914,565, filed on Oct. 14, 2019, Ser. No. 62/937,876, filed on Nov. 20, 2019, and Ser. No. PCT/US2020/036860, filed on Jun. 10, 2020, the entire said inventions being incorporated herein by reference.

### INCORPORATION BY REFERENCE OF SEQUENCE LISTING

The contents of the sequence listing text named “CRISPR_RNP_conjugate_CIP_2024_1”, which was created on Sep. 22, 2024, and 131,829 bytes in size, are incorporated herein by reference in its entirety.

### TECHNICAL FIELD OF THE INVENTION

The present invention relates to compositions of conjugates of a guide RNA(s)-Cas protein (RNP) complex and their uses as medicinal agents for treatment of viral infectious diseases and as gene regulation, disruption and/or correction-based therapeutics. The conjugate(s) comprises a guide RNA(s)-Cas protein (RNP) complex and one or more molecules selected from a group comprising PEG, non-PEG polymers, ligands for cellular receptors, lipids, oligonucleotides, antibodies, polysaccharides and peptides, and chemically linked to Cas protein and/or guide RNA(s). The guide RNA(s) is chemically modified to increase their stability, enhance the specificity for target recognitions and minimize/eliminate toxicities. The conjugates are delivered to targeted cells as RNP complexes, or formed in targeted cells from guide RNA conjugates and an mRNA or a plasmid or a viral vector encoding a Cas protein, or formed in targeted cells from a crRNA conjugate(s) and a plasmid or a viral vector encoding both a Cas protein and a tracrRNA. These conjugates are useful in improving preciseness in gene editing by driving templated DNA repair, in decreasing or preventing host preexisting immunity to guide RNA-Cas protein complexes by masking epitopes and chemical modifications of guide RNA(s), and also in improving the non-viral delivery of RNP complexes.

The present invention relates to compositions of conjugates of a guide RNA(s)-Cas protein (RNP) complex, wherein NLS sequence(s) is added to guide RNA conjugates instead of proteins to prevent the guide-independent off-target activities of Cas and Cas-effector fusion proteins (unbound by a targeting RNA moiety) in cell nuclei and enable controlled gene manipulations by controlled dosing of guide RNA-peptide conjugates.

The present invention further relates to a synthetic guide RNA-DNA template conjugate and its use in STAR editor for precise genome and/or epigenome editing, and the DNA template contains one or more 5-methyl cytosines. Also disclosed is the use of STAR editor for precise genome and/or epigenome editing as medicinal agents for treatment of diseases.

### BACKGROUND OF THE INVENTION

The following description of the background is provided simply as an aid in understanding the present disclosure and is not admitted to describe or constitute prior art to the present disclosure.

The CRISPR-Cas system is an adaptive immune system of bacteria and composed of clustered regularly interspaced short palindromic DNA repeats and CRISPR-associated genes that protect bacteria against invading phages and mobile genetic elements. CRISPR-Cas9 is being developed for numerous applications in biotechnology and biomedical research and as a gene therapy agent for treatment of multiple conditions including cancers, infectious diseases, and genetic diseases such as sickle cell anemia and Duchenne's muscular dystrophy (DMD), with 92 trials around the world that involve CRISPR in human cells listed in the NIH's database of global clinical trials to date. Using CRISPR-Cas9 multiplexing gene editing, allogenic universal CAR T cells that are deficient in the TCR beta chain, B2M, PD-1, TCR and CTLA-4 have been produced, with enhanced potency. CRISPR-Cas9 has been applied to silencing/correcting pathogenic proteins in neurodegenerative diseases such as Alzheimer's disease, Huntington's disease, and Parkinson's disease, which should essentially block further progression of symptoms, and it may also be applicable for treatment of dementia with Lewy bodies, frontotemporal dementias, various other tauopathies and amyotrophic lateral sclerosis (ALS). Catalytically impaired Cas9 (dCas9 and nCas9) can target many genomic loci, which has led to technological developments such as base editing, prime editing, epigenetic editing, gene regulation, and chromatin imaging and modeling.

Chronic and/or latent viral infections such as HIV, HBV, and HSV cause enormous suffering, life loss, and financial burdening among the infected individuals. These infectious diseases are incurable, and contagious to variable degrees, and are prominent threats for public health, highlighting the urgent needs for curative therapies. To date, effective antiviral therapies only suppress viral replication but do not clear virus in patients, and do not target the viral genetic materials of latently integrated (e.g. proviral DNA) or non-replicating episomal viral genomes (such as cccDNA) in human cells. Nevertheless, these viral DNAs have been reported to directly cause these chronic or latent infections.

Several reports showed CRISPR-Cas9 as potential antiviral treatments targeting viral genomes such as HBV, HIV, HSV, and Epstein-Barr virus (EBV). Yang et al. reported the CRISPR/Cas9 system could significantly reduce the production of HBV core and surface proteins in Huh-7 cells transfected with an HBV-expression vector, and disrupt the HBV encoding templates both in vitro and in vivo, indicating its potential in eradicating persistent HBV infection. They observed that two combinatorial gRNAs targeting different sites could increase the efficiency in causing indels. The study by Seeger and Sohn also reported CRISPR/Cas9 efficiently inactivated HBV genes in NTCP encoding HepG2 cells permissive for HBV infection. Wang and Quake observed patient-derived cells from a Burkitt's lymphoma with latent Epstein-Barr virus infection presented dramatic proliferation arrest and a concomitant decrease in viral load after exposure to a CRISPR/Cas9 vector targeted to the viral genome and a mixture of seven guide RNAs at the same molar ratio via plasmid. Hu et al. reported CRISPR/Cas9 system could eliminate the integrated HIV-1 genome by targeting the HIV-1 LTR U3 region in single and multiplex configurations. It inactivated viral gene expression and replication in latently infected microglial, promonocytic, and T cells, completely excised a 9,709-bp fragment of integrated proviral DNA that spanned from its 5′ to 3′ LTRs, and caused neither genotoxicity nor off-target editing to the host cells. CRISPR-Cas9 has been most recently shown to clear HIV-1 in a subset of humanized mice, when used in combination with long-acting slow-effective release antiretroviral therapy, promising a cure for this so-far incurable disease (Dash et al. Nat. Comm. 2019, 10, 2753). More studies can be found in a recent review on antiviral applications of CRISPR-Cas9 (Lee, C. Molecules 2019, 24, 1349).

As supported by previous studies using in vitro or in vivo transcribed crRNAs or sgRNAs, targeting genes or viral DNA at multiple sites could enhance the effectiveness, which can be better practiced by delivering a mixture/chemical library of different chemically modified crRNAs, sgRNAs or lgRNAs (including various spacers), targeting multiple sites and/or variants/mutations of a single site in viral genomes equivalent to combination therapies such as HAART.

Numerous diseases are caused by genetic alterations, other than mutations of DNA sequences, i.e., epigenetic alterations such as DNA methylation. Genetic base sequences (exons) encode proteins, while cell type-specific properties, such as gene expression, are encoded at both a genetic sequence and an epigenetic level including DNA methylation. Genetic sequence mutations have been compiled in literature, and their corrections have been explored for cure of the corresponding diseases. Epigenetics is less advanced, but many diseases, such as neurodegenerative diseases including Alzheimer's, aging, cancers and substance addictions have found causal correlations with distinct epigenetic changes.

DNA methylation involves the covalent transfer of a methyl group from S-adenosyl-methionine (SAM) to the C-5 position of cytosine to form 5-methylcytosine (m5C). DNA methylation is mediated by DNA methyltransferases (DNMT) and proteins involved in DNA demethylation (TET and etc.), and plays a critical role in numerous biological processes including transcription regulation, chromosome maintenance, and genomic imprinting.

One such example is hepatitis B. HBV Infection is a world-wide health problem with over 300 million of chronically infected patients, causing hepatitis B, cirrhosis and hepatocellular carcinoma (HCC). The mini-chromosome of HBV and integrated HBV DNA includes three putative CpG islands. It was reported that the methylation status of one of the CpG islands (CpG III) significantly correlates with hepatocarcinogenesis (Su et al. 2015). In addition, epigenetic modifications have been implicated as regulatory mechanisms of cccDNA transcription. Methylation status of CpG II was reported to correlate with transcription of cccDNA, HBeAg status and serum viral DNA levels, and CpG II is located near the core promoter (Guo et al. 2017).

Another such example is Alzheimer's disease (AD). Aberrant DNA methylation occurs in many AD susceptibility genes including the amyloid precursor protein gene (APP). A large CpG island is located in the promoter region of APP. APP encodes amyloid precursor protein, a key protein involved in Alzheimer's disease (AD) pathogenesis. Hypomethylation of APP gene was detected in AD patients, and was suggested to control AD pathogenesis.

Therapeutic treatments for diseases by epigenetic approaches include small molecules and potentially epigenome editing. Small molecule drugs targeting epigenome by inhibiting chromatin-structure modifying enzymes affect the entire genome, leading to substantial side cytotoxicity in patients. Epigenome editing targeting only one or a few DNA sequences in genome promises a significant decrease in such toxicity. In addition, chromatin structures modified by epigenome editing were reported to remain inherited after hundred times of cell division, due to the endogenous epigenetic maintenance mechanism within cells.

It is well known the DNA methylation is maintained in cellular DNA synthesis; the methylation pattern of parental DNA is faithfully copied to the newly formed daughter strand by cellular protein effectors such as UHRFI and DNMT1. After the formation of a double stranded break, DNA repair pathways of both NHEJ and HDR were suggested to contribute to maintenance of DNA methylation patterns inheritable through numerous cell division cycles. An exogenous methylated long dsDNA template (1.4 Kb) was used in combination with Cas9 to produce stably methylated cell lines, though short templates (190 nt ssDNA and dsDNA) showed generated methylation were not maintained through cell divisions and thus not possibly applicable for therapeutic epigenome editing. Consequently, epigenome editing may be suitable for the treatment of dominant genetic diseases, such as those caused by gain-or loss-of-function type epigenomic alterations.

Various genetic editors towards precise DNA manipulations have been developed or in development, including base editors (BE) and prime editor (PE) and epigenome editors (CRISPRon/CRISPRoff). These editors comprise a Cas protein fused with one or more effector proteins such as nucleotide deaminases, reverse transcriptase (error-prone) and DNA methyl transferases (DNMT) or proteins involved in DNA demethylation (TET and etc.). In addition to off-targets caused by tolerated mismatches between the spacer and protospacer sequences, the activities of these fused effector proteins are independent of guide-directed target recognitions, and cause guide-independent off-targets. Another challenge is the lack of single nucleotide-resolution (because of editing windows), which makes both base editors and epigenome editors imprecise. In addition, the protospacer is unavailable for editing by both base editors and epigenome editors.

The fusion protein used in these editors present an insurmountable challenge in viral delivery, in particular, by an AAV viral vector (with a packing capacity of less than 4.7 kbp). AAVs are ideal delivery systems for gene editors because of their low immunogenicity, high serotype abundance, ability to preferentially infect specific tissues and are used in clinic for gene therapy.

STAR editor uses guide RNA-ssDNA conjugates and a DNA-directed DNA polymerase for precise gene editing (See, e.g., US Patent Publication US 2021/0054371, the entire disclosures of which are incorporated herein by reference.). The DNA polymerase(s) can be endogenous, and thus no fusion proteins of a Cas (active, partial active or in active) with a DNA polymerase are required though the fusion protein has certain advantages such as being encoded by a single nucleic acid if the packaging size in a delivery vehicle is not a limit, e.g. by a non-viral vector. In comparison to guide-independent deamination function of fused DNA deaminases and error-prone reverse transcriptase (lack of proofreading), and because the needed editing is integrated in its tethered DNA template, STAR editor is a superior precise DNA sequence editor.

By arbitrary installation 5-methyl cytosines in its DNA template, STAR editor is capable of nucleotide-specific methylation or demethylation particularly valuable for epigenome editing at single CpG dinucleotide resolution level, which is critical for deconvolution of the synergic effects of CpG methylations and lowering the risk of regional or genome-wide epigenome editing, which disrupts numerous biological processes potentially leading to serious adverse events (SAE) in therapeutic treatments. This invention further pertains to a synthetic guide RNA-DNA template conjugate and its use in STAR editor for both genome editing and precise epigenome editing, and the DNA template contains absent, one or more 5-methyl cytosines (The DNA template can be either hypomethylated or hypermethylated.). With this novel nucleic acid conjugate, STAR editor is both a precise DNA sequence editor and a precise epigenome editor. In contrast to reported HDR-based approaches to install methylations, which requires hardly-accessible long DNA templates position-specifically substituted with 5-methyl cytosines, STAR editor-based epigenome editing takes a multiplexing approach with short methylated DNA templates.

This invention further pertains to the use of STAR editor and its conjugates for genome editing and precise epigenome editing as medicinal agents for treatment of diseases.

### SUMMARY OF THE INVENTION

This invention pertains to compositions of conjugates of a guide RNA(s)-Cas protein (RNP) complex and their uses as medicinal agents in treatment of viral infectious diseases and as gene regulation, disruption and/or correction-based therapeutics. The conjugate(s) comprises a guide RNA(s)-Cas protein (RNP) complex and one or more molecules selected from the group comprising PEG, non-PEG polymers, ligands for cellular receptors, lipids, oligonucleotides, antibodies, polysaccharides and peptides, and chemically linked to Cas protein and/or guide RNA(s). The conjugates are delivered to targeted cells as RNP complexes, or formed in targeted cells from guide RNA conjugates and an mRNA or a plasmid or a viral vector encoding a Cas protein delivered by co-injections or separate injections, or formed in targeted cells from crRNA conjugate(s) and a plasmid or a viral vector encoding both a Cas protein and a tracrRNA delivered by co-injections or separate injections. These conjugates are useful in improving preciseness in gene editing by driving templated DNA repair, in decreasing or preventing host preexisting immunity to guide RNA(s)-Cas protein complexes by masking epitopes and chemical modifications of guide RNA(s), and also in improving the non-viral delivery of RNP complexes.

The present invention relates to compositions of conjugates of a guide RNA(s)-Cas protein (RNP) complex, wherein NLS sequence(s) is added to guide RNA conjugates instead of proteins to prevent the guide-independent off-target activities of Cas and Cas-effector fusion proteins (unbound by a targeting RNA moiety) in cell nuclei and enable controlled gene manipulations by controlled dosing of guide RNA-peptide conjugates.

The guide RNA(s) of said RNP complex conjugates is a chemically modified crRNA, dual guide RNAs (crRNA and tracrRNA), a sgRNA or a lgRNA oligonucleotide comprising nucleotides modified at sugar moieties such as 2′-deoxyribonucleotides, 2′-methoxyribonucleotides, 2′-F-ribonucleotides, 2′-F-arabinonucleotides, 2′-0,4′-C-methylene nucleotides (LNA), unlocked nucleotides (UNA), nucleoside phosphonoacetates (PACE), thiophosphonoacetates (thioPACE), and phosphoromonothioates:

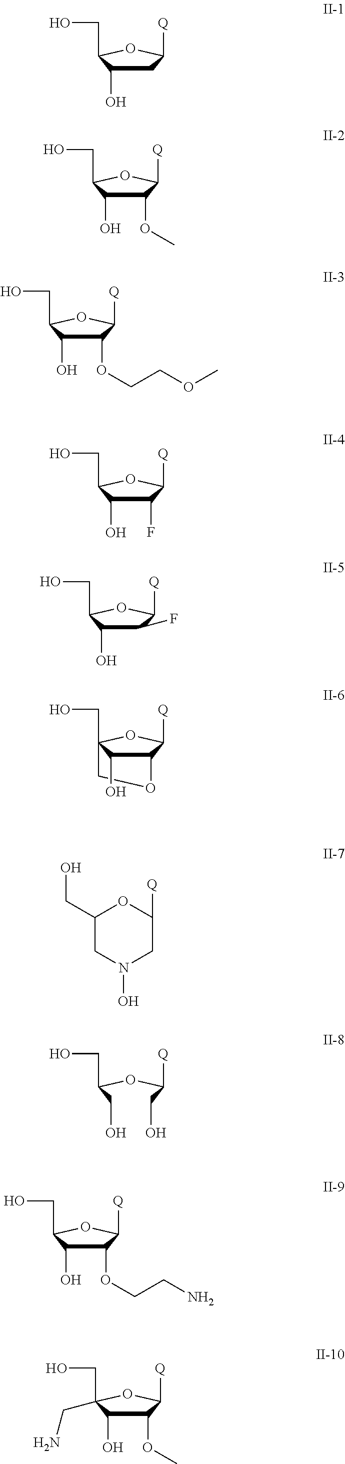

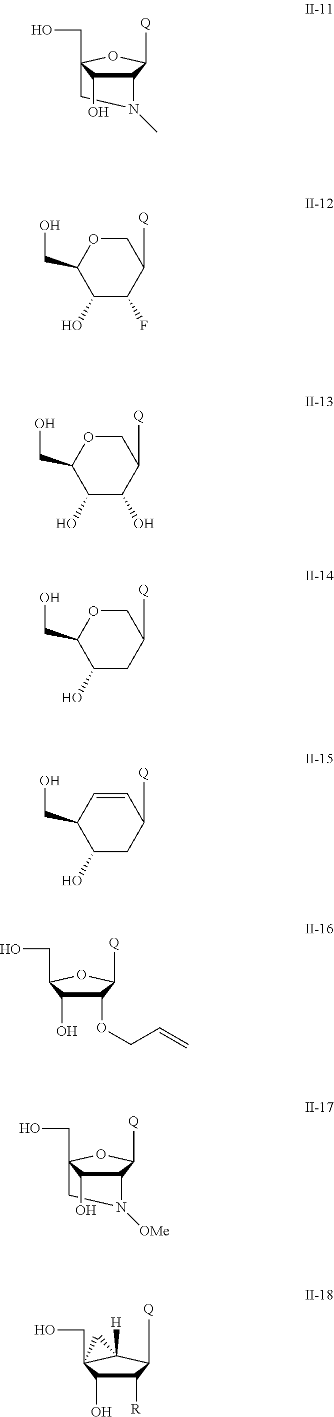

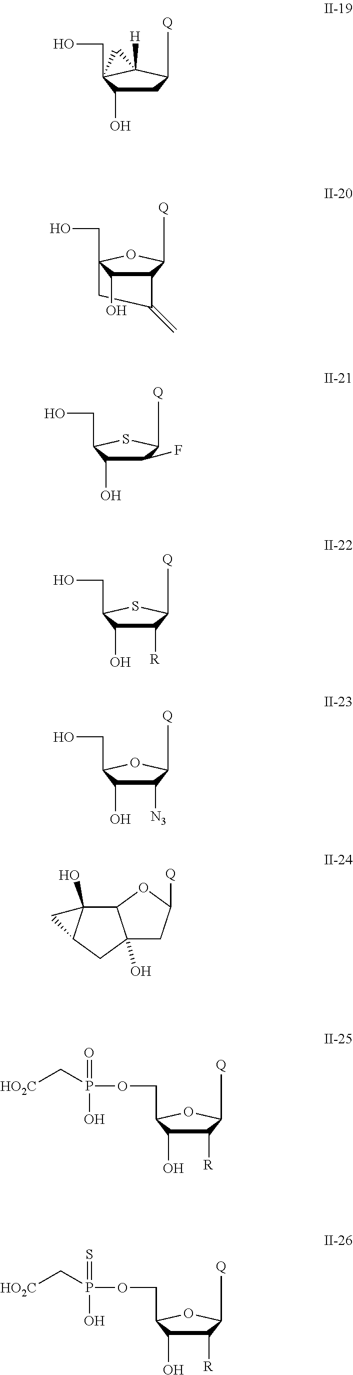

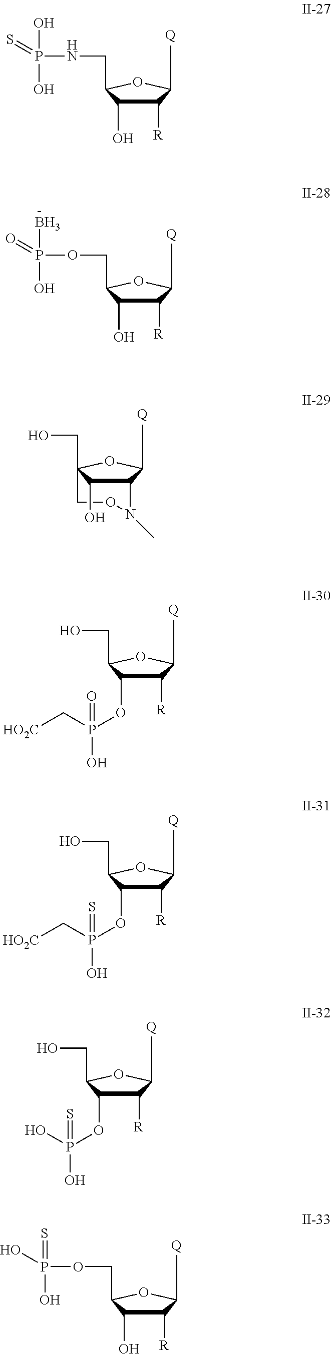

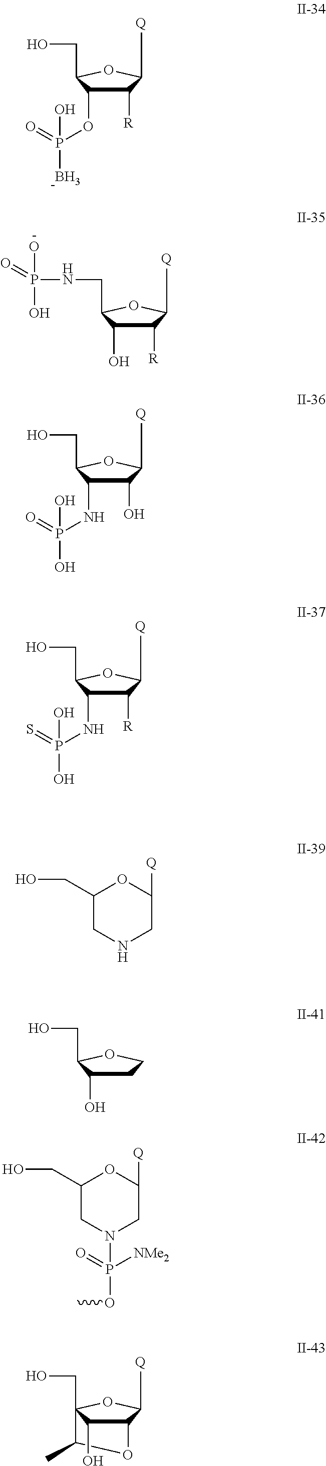

wherein Q is a nucleobase and R is H, OH, F, OMe, or OCH₂CH₂OCH₃; The chemically modified crRNA, sgRNA or lgRNA oligonucleotides optionally comprise modified nucleotide base moieties such as G-clamps, A-clamps and other modified bases:

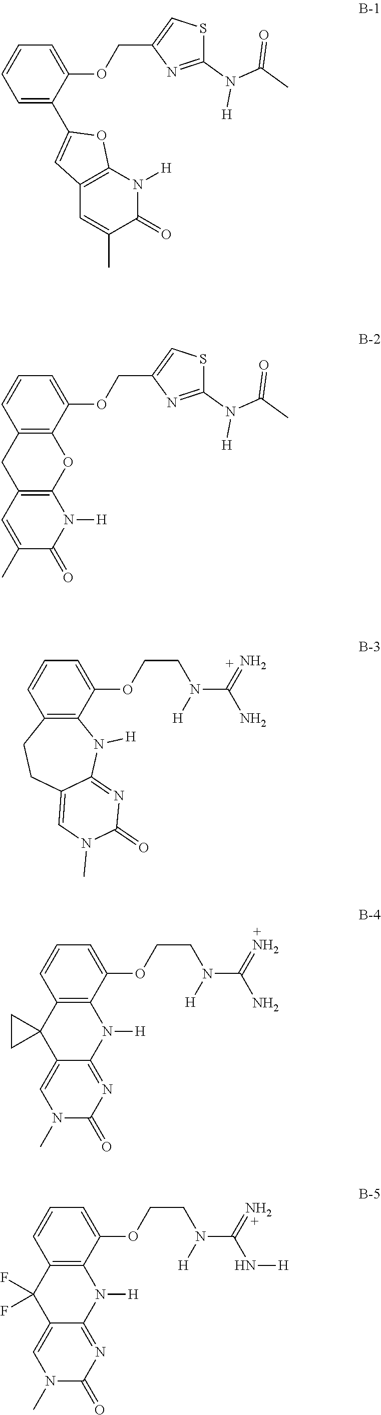 [SMILES: CC(=O)Nc1nc(COc2ccccc2-c2cc3cc(C)c(=O)[nH]c3o2)cs1 ; CC(=O)Nc1nc(COc2cccc3c2Oc2[nH]c(=O)c(C)cc2C3)cs1 ; Cn1cc2c(nc1=O)Nc1c(cccc1OCCNC(=N)N)CC2 ; Cn1cc2c(nc1=O)Nc1c(OCCNC(=N)N)cccc1C21CC1 ; Cn1cc2c(nc1=O)Nc1c(OCCNC(=N)N)cccc1C2(F)F]

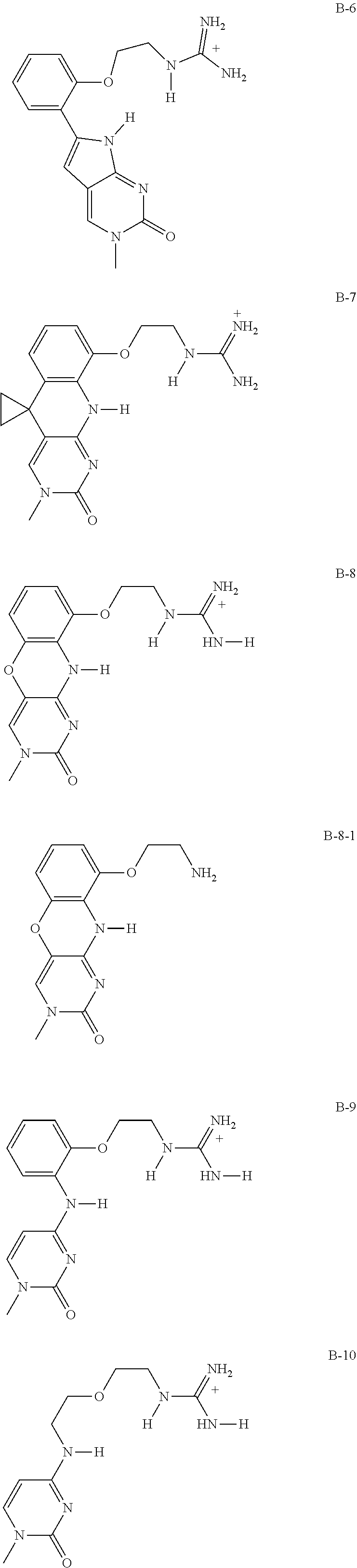 [SMILES: Cn1cc2cc(-c3ccccc3OCCNC(=N)N)[nH]c2nc1=O ; Cn1cc2c(nc1=O)Nc1c(OCCNC(=N)N)cccc1C21CC1 ; Cn1cc2c(nc1=O)Nc1c(OCCNC(=N)N)cccc1O2 ; Cn1cc2c(nc1=O)Nc1c(OCCN)cccc1O2 ; Cn1ccc(Nc2ccccc2OCCNC(=N)N)nc1=O ; Cn1ccc(NCCOCCNC(=N)N)nc1=O]

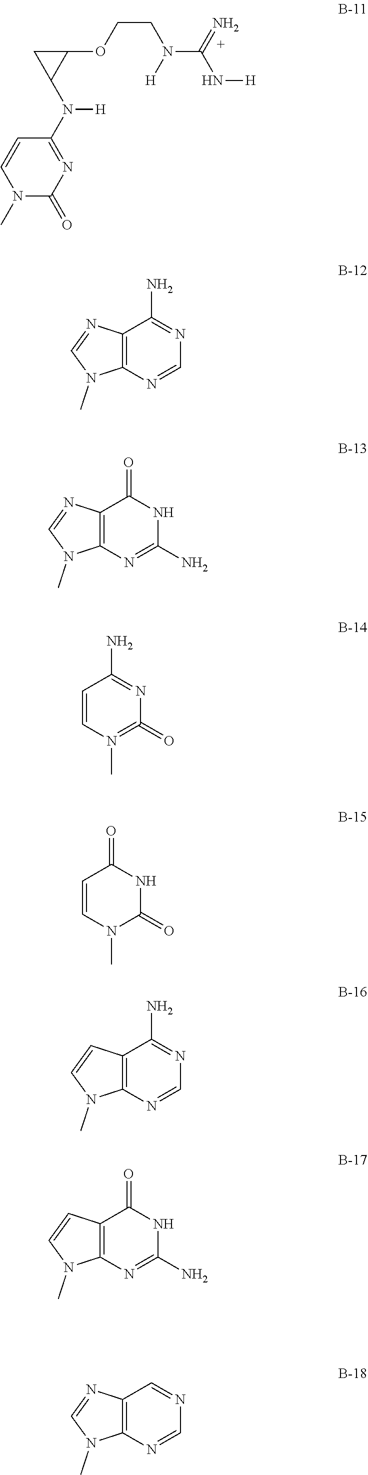

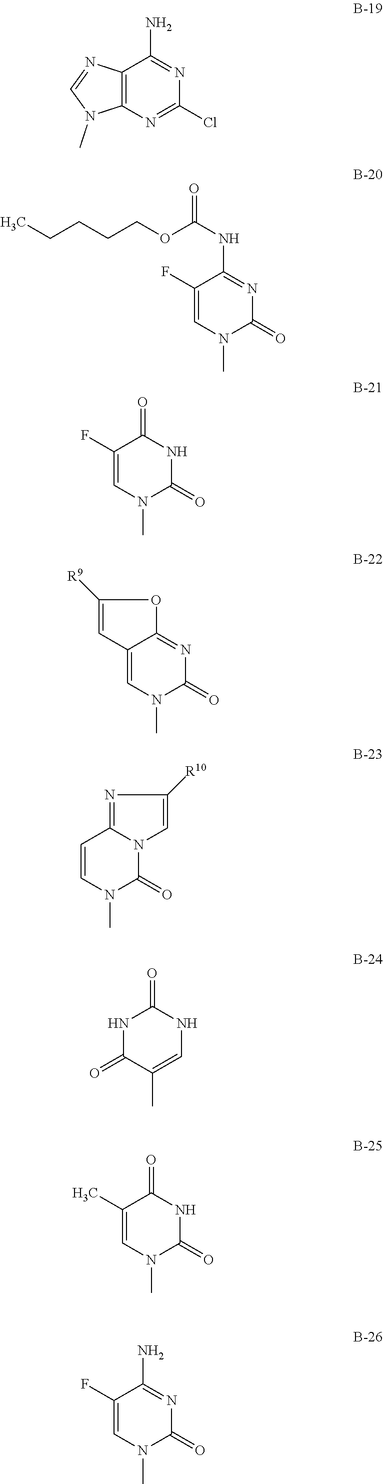

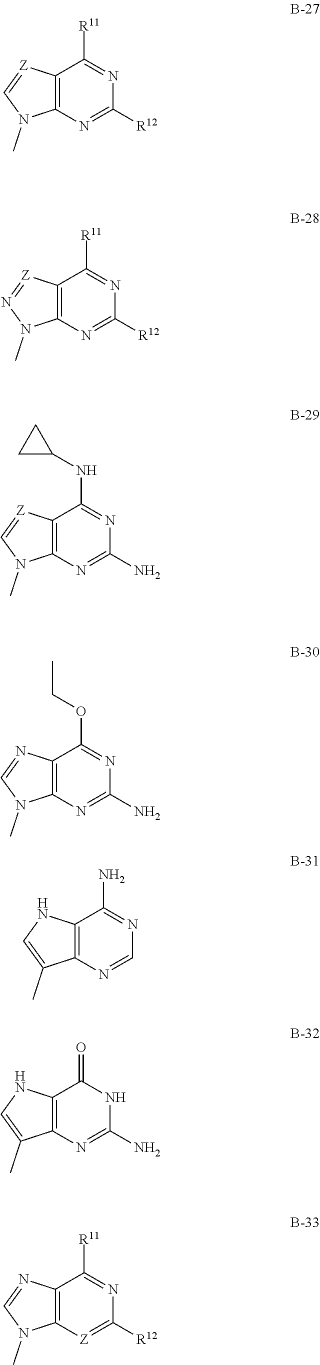

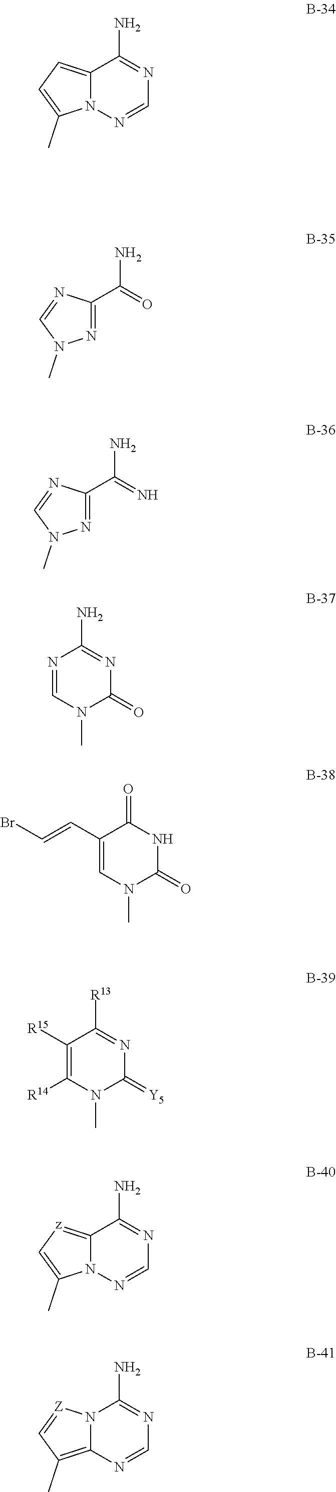

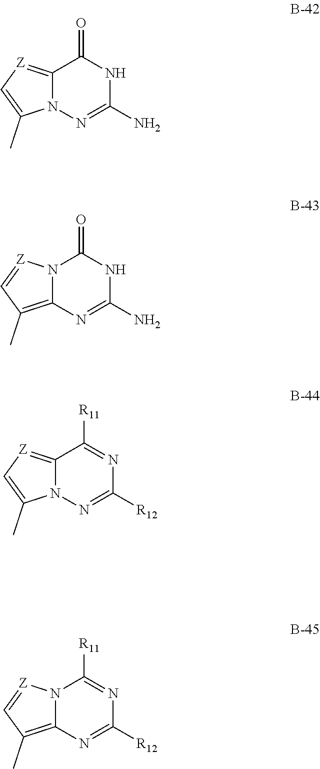

wherein:
(i) Z is N or CR¹⁶; (ii) R⁹, R¹⁰, R¹¹ , R¹², R¹³, R¹⁴, R¹⁵ and R¹⁶ are independently H, F, Cl, Br, I, OH, OR′, SH, SR′, SeH, SeR′, NH₂, NHR′, NHOH, NHOR′, NR′OR′, NR′₂, NHNH₂, NR′NH₂, NR′NHR′, NHNR′₂, NR′NR′₂, lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, CN, CO₂H, CO₂R′, CONH₂, CONHR′, CONR′₂, CH—CHCO₂H, or CH═CHCO₂R′, wherein R′is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, chemical modifications of guide RNAs at either sugar or base moiety or both optimize the complementary recognition of the guide-target duplex to improve the cutting efficiency and lower the off-target effects.

In some embodiments, chemical modifications of guide RNAs at either sugar or base moiety or both optimize the shape complementarity between Cas protein and the minor and major grooves of the guide-target duplex to improve the efficiency and lower the off-target effects.

In some embodiments, the chemically modified crRNA, dual guide RNAs, sgRNA or lgRNA oligonucleotide is conjugated with peptides, aptamers, oligonucleotides, antibodies, small molecule receptor ligands such as GalNAc, biotin, cholesterol, tocopherol, lipid, or folate and etc., for selective tissue targeting. The conjugating sites are selected from 3′-end, 5′-end and ligation sites of these oligonucleotides.

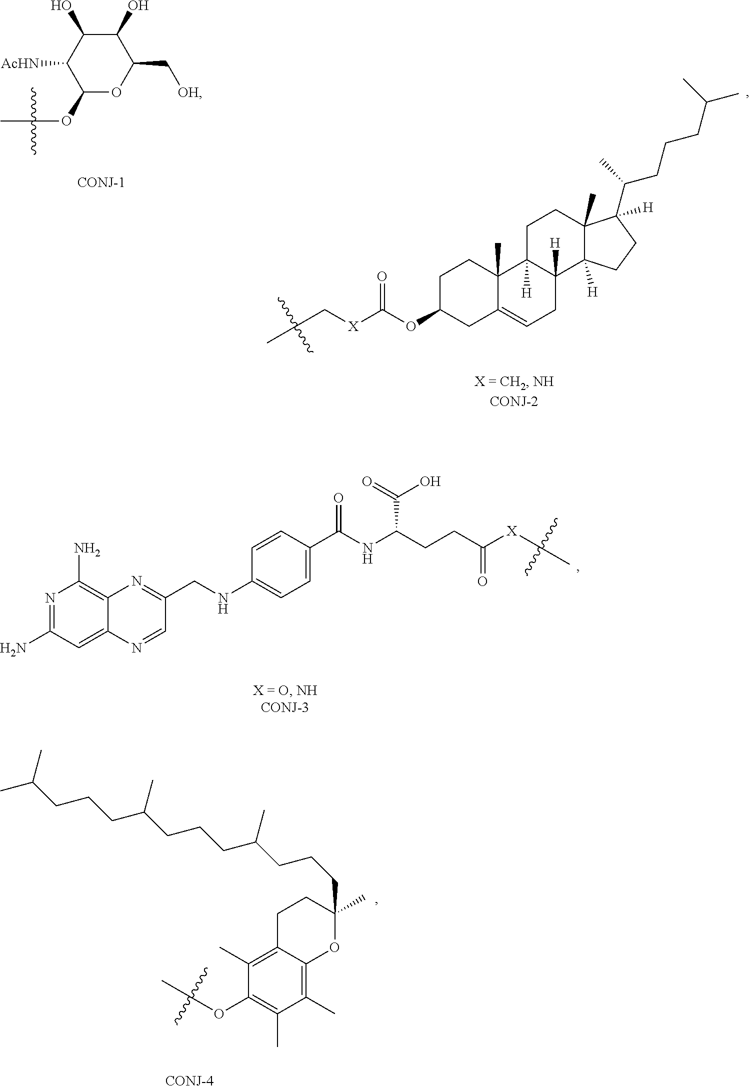

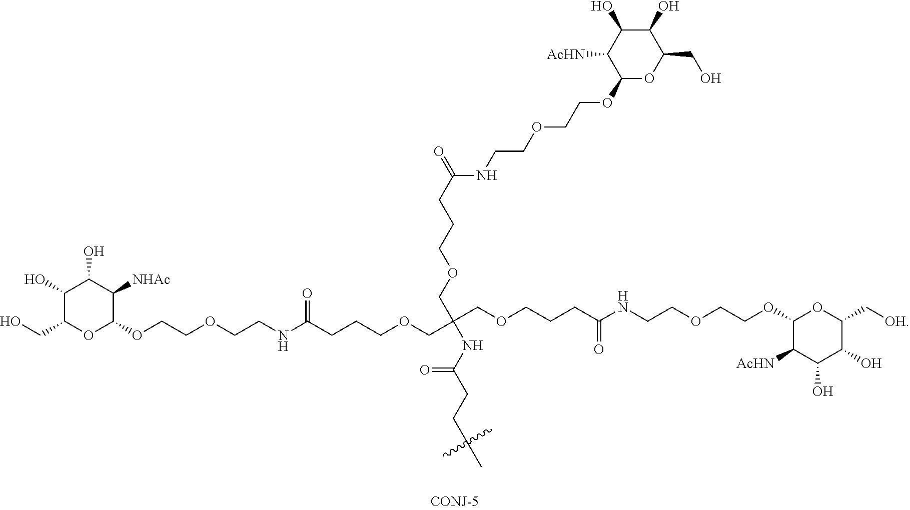 [SMILES: *CCC(=O)NC(COCCCC(=O)NCCOCCO[C@@H]1O[C@H](CO)[C@H](O)[C@H](O)[C@H]1NC(C)=O)(COCCCC(=O)NCCOCCO[C@@H]1O[C@H](CO)[C@H](O)[C@H](O)[C@H]1NC(C)=O)COCCCC(=O)NCCOCCO[C@@H]1O[C@H](CO)[C@H](O)[C@H](O)[C@H]1NC(C)=O]

In certain embodiments, the said viral vectors encoding both a Cas protein and a tracrRNA comprise the following elements, optionally in 5′>3′ orientation: a mammalian promoter and optional enhancer, a cDNA encoding a single Cas protein, one or more nuclear localization sequence, a polyadenylation signal, a U6 promotor and the tracrRNA sequence. As an example, an adeno-associated virus (AAV) vector is depicted in FIG. 7 (A).

In certain embodiments, the said viral vectors encoding both a Cas protein (lacking NLS) and a tracrRNA comprise the following elements, optionally in 5′>3′ orientation: a mammalian promoter and optional enhancer, a cDNA encoding a single Cas protein, a polyadenylation signal, a U6 promotor and the tracrRNA sequence.

In certain embodiments, the said viral vectors enabling targeted cells to stably express Cas comprise the following elements, optionally in 5′>3′ orientation: a mammalian promoter and optional enhancer, a cDNA encoding a single Cas protein, one or more nuclear localization sequence, and a polyadenylation signal. As an example, an adeno-associated virus (AAV) vector is depicted in FIG. 7 (B).

In certain embodiments, the said viral vectors enabling targeted cells to stably express Cas (lacking NLS) comprise the following elements, optionally in 5′>3′ orientation: a mammalian promoter and optional enhancer, a cDNA encoding a single Cas protein, and a polyadenylation signal.

In some embodiments, the Cas protein is a Cas9.

In some embodiments, the cDNAs encoding Cas9 and/or tracrRNA of the said viral vectors are optimized to encode Cas9 variants and tracrRNA for both better efficacy and lower off-target and other side effects.

In some embodiments, the said viral vectors and crRNA(s), or lgRNAs or sgRNAs or their conjugates either in an aqueous solution with or without transfection reagents or packaged in a non-viral carrier, are administrated by co-injections or separate injections.

In some embodiments, the conjugates of functional ternary crRNA-tracrRNA-Cas9 complexes are formed cellularly or in vivo by either tracrRNA-Cas9 binary complex formation followed by hybridization of crRNA or crRNA conjugates to the bound tracrRNA via repeat:anti-repeat recognition and interactions with Cas9, or binding of Cas9 to a crRNA-tracrRNA complex or its conjugates dimerized via repeat:anti-repeat recognition. The conjugates of functional binary lgRNA/sgRNA-Cas9 complexes are formed in vivo by binding of stably or inducibly expressed Cas9 to dosed lgRNA or sgRNA conjugates.

In some embodiments, the conjugates of a guide RNA(s)-Cas protein (RNP) complex, wherein NLS sequence(s) is added to guide RNA conjugates instead of proteins to prevent the guide-independent off-target activities of Cas and Cas-effector fusion proteins (unbound by a targeting RNA moiety) in cell nuclei and enable controlled gene manipulations by controlled dosing of guide RNA-peptide conjugates, are formed cellularly or in vivo by binding of stably or inducibly expressed proteins or tracrRNA-Cas9 binary complexes to dosed crRNA-NLS, lgRNA-NLS or sgRNA-NLS conjugates.

In certain embodiments, arrayed libraries of structurally optimized chemically modified lgRNAs or crRNAs or their conjugates and cells or animals stably or inducibly expressing a Cas protein or a Cas9-tracrRNA binary complex are used for drug discovery, medical research and biological studies, and genome wide screening.

In some embodiments, the Cas protein is a single protein effector of other class 2 CRISPR systems, such as a Cas12a protein, which is delivered in a tissue tropic viral vector, and chemically modified crRNA(s) or its conjugates are delivered either in an aqueous solution gymnotically or with transfection reagents or packaged in a non-viral carrier by co-injections or separate injections.

In some embodiments, the single protein effector such as Cas9 and Cas12a is catalytically inactive and coupled/fused with protein effectors such as transcription activators, transcription repressors, catalytic domains of DNA methyltransferase, histone acetyltransferase and deacetylase, reverse transcriptase, and nucleic acid deaminases, for gene editing and regulations.

In some embodiments, the said vectors and oligonucleotides or oligonucleotide conjugates are administrated to the target cells, i.e. T cells from patients, and the modified cells are infused back to the patients.

In another embodiment, the target tissue or cells are treated with the said vectors encoding a tracrRNA-Cas9 binary complex, and the modified cells are infused back to the patients. The said crRNAs or their conjugates are administrated to activate the CRISPR-Cas9 for regulation, disruption or correction of targeted genes or deactivation of viral genomes.

In yet another embodiment, the target tissue or cells are treated with the said vectors encoding the Cas9 protein, and the modified cells are infused back to the patients. The said lgRNAs or sgRNAs or their conjugates are administrated to activate the CRISPR-Cas9 for regulation, disruption or correction of targeted genes or viral genomes.

In some embodiments, a viral vector encoding a Cas9 orthologue has the tracrRNA encoded in cis, and crRNA(s) or crRNA conjugates are administrated either in an aqueous solution with or without transfection reagents, or packaged in a non-viral carrier, are administrated by co-injections or separate injections with administration ratios of the vector to copies of each crRNA ranging from 1:1 to 1:5. In some embodiments, a single crRNA or its conjugate is administrated. In some embodiments, a mixture of multiple crRNAs or their conjugates is administrated.

In some embodiments, a viral vector encoding a Cas9 orthologue, and lgRNA or lgRNAs or their conjugates either in an aqueous solution with or without transfection reagents, or packaged in a non-viral carrier, are administrated by co-injections or separate injections with administration ratios of the vector to copies of each lgRNA ranging from 1:1 to 1:5.

In certain embodiments, the viral vector is selected from engineered adeno-associated virus (AAV), retrovirus, lentivirus, adenovirus vehicles, etc.

In certain embodiments, the codons for Cas9 protein are codon-optimized for human cells.

In certain embodiments, the codons for Cas9 protein bearing a C-terminal SV40 nuclear localization signal are codon-optimized for human cells.

In certain embodiments, the expression of Cas9 protein is under the control of a single or a plurality of switchable transcription promotors/enhancers/depressors.

In certain embodiments, a Cas9 protein is selectively delivered to specific tissues based on tissue tropism of the viral vector and cell selective promotor of Cas9 gene.

In certain embodiments, crRNA, dual guide RNAs, sgRNA or lgRNA comprises a spacer selected from sequences of 12˜20 nt of DNA genome of a pathogen, e.g. a virus, bacterium, or other microorganism that causes disease(s), of which each thymine is replaced with uracil, immediately next to a PAM (such as NGG for SpCas9). The spacer RNA oligomer has the same sequence as the sense non-target strand (5′->3′) of the genome immediately next to the protospacer adjacent motif (PAM), and namely is its RNA transcript with or without further chemical modifications. The recognition of the complementary anti-sense target DNA strand by crRNA, sgRNA or lgRNA directs Cas DNA endonuclease for site-specific cleavage to form a specific double strand break (DSB) leading to degradation of viral genomes or deadly mutations for the pathogen resulting from DNA repair pathways via non-homologous end joining (NHEJ) and microhomology mediated end joining (MMEJ).

In certain embodiments, crRNA, sgRNA or lgRNA comprises a spacer selected from sequences of 12˜20 nt of HIV genomes, of which each thymine is replaced with uracil, immediately next to a PAM (such as NGG). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer selected from sequences of 12˜20 nt of HBV genomes, of which each thymine is replaced with uracil, immediately next to a PAM (such as NGG for SpCas9). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer selected from sequences of 12˜20 nt of HSV genomes, of which each thymine is replaced with uracil, immediately next to a PAM (such as NGG for SpCas9). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer selected from sequences of 12˜20 nt of EBV genomes, of which each thymine is replaced with uracil, immediately next to a PAM (such as NGG for SpCas9). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer sequence selected from sequences next to a PAM sequence in the antisense strand of target genomes.

In certain embodiments, crRNAs, sgRNAs and lgRNAs comprise different spacers corresponding to different loci of viral genomes and/or variants of a single locus of target genomes.

In certain embodiments, crRNAs, sgRNAs and lgRNAs comprise spacers selected from sequences of 12˜20 nt of host genes encoding host factors involved in viral entry, transcription/reverse transcription and/or replications, of which each thymine is replaced with uracil, immediately next to a PAM (such as NGG for SpCas9). The spacer RNA oligomers have the same sequences as the sense non-target strands (5′→3′) of the genomes immediately next to the protospacer adjacent motif (PAM), and namely are their RNA transcripts with or without further chemical modifications. The recognition of the complementary anti-sense target DNA strand by crRNAs, sgRNAs or lgRNAs directs Cas DNA endonuclease for site-specific cleavage to form a specific double strand break (DSB), introducing mutations in these factors leading to host's resistance to the virus.

In certain embodiments, crRNAs, sgRNAs and lgRNAs comprise spacers selected from sequences of 12˜20 nt of host mutated loci, defective gene or a target gene encoding a protein of defective or partial activity or function, of which each thymine is replaced with uracil, immediately next to a PAM (such as NGG for SpCas9). The spacer RNA oligomers have the same sequences as the sense non-target strands (5′→3′) of the genomes immediately next to the protospacer adjacent motif (PAM), and namely are their RNA transcripts with or without further chemical modifications. The recognition of the complementary anti-sense target DNA strand by crRNAs, sgRNAs or lgRNAs directs Cas DNA endonuclease for site-specific cleavage to form a specific double strand break (DSB), allowing a transgene cassette flanked with homologous regions to recombine with the host loci and replace the mutated DNA with the correct sequence.

In certain embodiments, the said transgene cassette is contained in an ssDNA HDR template conjugated to guide RNAs to form a guide RNA(s)-ssDNA conjugate for replacing a mutated DNA with its correct sequence.

In certain embodiments, the said transgene cassette is contained in an ssDNA HDR template conjugated to guide RNAs for editing viral episomal DNAs and integrated viral DNAs in host genes, and the editions are deletions, insertions or point mutations to suppress or eliminate viral protein expression, or overexpression of host pathogenic proteins upregulated by viral DNA integration.

In certain embodiments, the said editions incorporate stop codon(s) and/or cis regulatory elements to suppress or eliminate viral protein expression, or overexpression of host pathogenic proteins upregulated by viral DNA integration.

In certain embodiments, the said guide RNA(s)-ssDNA conjugates are optionally further conjugated with peptides, aptamers, antibodies, small molecule receptor ligands such as GalNAc, cholesterol, tocopherol, lipids, or folate, etc., for selective tissue targeting. The conjugating sites are selected from 3′-end, 5′-end and ligation sites of these said guide RNA(s)-ssDNA conjugates.

The Cas protein is selected from Cas9 variants comprising SpCas9, St1Cas9, SaCas9, NmCas9, etc. (Jin et al. Adv. Sci. 2020, 1902312; Doudna, J. A. Nature 2020, 578, 229), and can be a nickase or catalytically inactive Cas9 (dCas9) coupled/fused with protein effectors such as transcription activators, transcription repressors, catalytic domains of DNA methyltransferase, histone acetyltransferase and deacetylase, reverse transcriptase and nucleic acid deaminases, for gene editing and regulations.

The Cas protein can be alternatively any single protein effector of other class 2CRISPR systems (Type V and VI), such as a Cas12 (a, b, c, e, g, h, i, etc.), Cas13 and Cas14 protein. The single protein effector such as Cas12 and Cas14 can be catalytically inactive and coupled/fused with protein effectors such as transcription activators, transcription repressors, catalytic domains of DNA methyltransferase, histone acetyltransferase and deacetylase, and nucleic acid deaminases, reverse transcriptase, for gene editing and regulations.

In certain embodiments, the Cas protein is optionally engineered to introduce conjugating cysteines to replace selected solvent exposed amino acids near to or contained by epitopes by site directed mutagenesis, and cysteines of wild type enzymes (e.g. C80 and C573 of SpCas9) are optionally mutated to avoid potential deactivation of enzymes due to conjugations at these cysteines.

In certain embodiments, the guide RNA(s)-Cas protein (RNP) complex(es) is conjugated with molecules selected from PEG, non-PEG polymers, ligands for cellular receptors, antibodies, lipids, oligonucleotides, polysaccharides, glycans and peptides.

In certain embodiments, the Cas conjugating sites are selected from surface/solvent exposed/protruded amino acid residues such as lysine, arginine, serine, cysteine, aspartate, or glutamate of Cas proteins or an amino acid(s), e.g. a cysteine, introduced by site-directed mutagenesis for selective conjugations to mask or shield epitopes.

In certain embodiments, the guide RNA(s)-Cas protein (RNP) complex(es) is conjugated at either Cas protein, or guide RNAs, or both.

In certain embodiments, the guide RNA(s)-Cas protein (RNP) complex(es) is PEGylated.

In certain embodiments, the guide RNA(s)-Cas protein (RNP) complex(es) is PEGylated with more than two PEG polymers to shield/mask epitopes.

In certain embodiments, the guide RNA(s)-Cas protein (RNP) complex(es) are conjugated with other non-PEG polymers, ligands for cellular receptors, lipids, oligonucleotides, antibodies, polysaccharides, glycans or peptides.

In certain embodiments, PEG-conjugated guide RNA-Cas protein (RNP) complex(es) is further covalently linked to other non-PEG polymers, ligands for cellular receptors, lipids, oligonucleotides, antibodies, polysaccharides, glycans or peptides.

In certain embodiments, guide RNA(s)-Cas protein (RNP) complex(es) is covalently linked to one or more molecules selected from PEG, non-PEG polymers, ligands for cellular receptors, lipids, oligonucleotides, antibodies, polysaccharides, glycans and peptides.

In certain embodiments, guide RNA(s)-conjugates, e.g. lgRNA-ssDNA conjugates, are delivered with an mRNA or a vector encoding a Cas protein.

In certain embodiments, guide RNA(s)-conjugates, e.g. lgRNA-ssDNA conjugates, are delivered to cells stably or inducibly expressing a Cas protein.

In certain embodiments, a Cas protein or Cas protein/tracrRNA encoded by an mRNA, a plasmid or a viral vector in eukaryotic cells does not have NLS.

In certain embodiments, a Cas protein or Cas protein/tracrRNA encoded by an mRNA, a plasmid or a viral vector in mammalian cells does not have NLS.

In certain embodiments, conjugates of crRNAs, sgRNAs and lgRNAs comprise one or more NLS peptides.

In certain embodiments, NLS conjugates of sgRNAs and lgRNAs form RNP complexes in cells with a Cas protein comprising no NLS.

In certain embodiments, NLS conjugates of crRNAs form RNP complexes in cells with a Cas protein lacking NLS and bound by a tracrRNA.

In certain embodiments, NLS conjugates of guide RNAs form RNP complexes in cells with a Cas-effector fusion protein comprising no NLS.

In certain embodiments, lgRNA-ssDNA conjugates are used to drive the templated repair of the cleaved/nicked DNA upon hybridization of ssDNA with the PAM distal fragment (primer) of the R-loop asymmetrically released from Cas9:lgRNA:DNA complexes, and thus to promote precise editing and fast release of the RNP complex from the edited DNA to increase its turnover frequency (TOF).

In certain embodiments, lgRNA-ssDNA conjugates are used to drive the templated repair of the cleaved/nicked DNA upon hybridization of ssDNA with the PAM distal fragment (primer) of the R-loop asymmetrically released from Cas9:lgRNA:DNA complexes to form a complementary duplex of 7-14 nt upstream and before a PAM proximal sequence (6-13 nt including cutting site, 5′→3′), the complementarity between the template for chain extension by a DNA-directed DNA polymerase and the PAM proximal sequence (including PAM) is minimized by silent mutations (e.g., replacement with alternative codons), and thus to promote precise editing and fast release of the RNP complex from the edited DNA to increase its turnover frequency (TOF).

In certain embodiments, lgRNA-ssDNA conjugates are used to drive the templated repair of the cleaved/nicked DNA upon hybridization of ssDNA with the PAM distal fragment (primer) of the R-loop asymmetrically released from nCas9 (H840A):lgRNA:DNA complexes to form a complementary duplex of 7-14 nt upstream and before a PAM proximal sequence (6-13 nt including cutting site, 5′→3′), the complementarity between the template for chain extension by a DNA-directed DNA polymerase and the PAM proximal sequence (including PAM) is minimized by silent mutations (e.g., replacement with alternative codons), and thus to promote precise editing and fast release of the RNP complex from the edited DNA to increase its turnover frequency (TOF).

In certain embodiments, lgRNA-ssDNA conjugates are used to drive the templated repair of the cleaved/nicked DNA upon hybridization of ssDNA with the 3′-OH fragment of the R-loop in Cas:lgRNA:DNA complexes, and thus to promote precise editing and fast release of the RNP complex from the edited DNA to increase its turnover frequency (TOF).

In certain embodiments, a synthetic guide RNA-ssDNA template conjugate and its use in STAR editor for precise epigenome editing, and the DNA template contains absent, one or more 5-methyl cytosines (The DNA template can be either hypomethylated or hypermethylated.). With this novel nucleic acid conjugate, STAR editor is both a precise DNA nucleotide sequence editor and a precise epigenome editor. In contrast to reported HDR-based approaches to install methylations, which requires hardly-accessible long DNA templates position-specifically substituted with 5-methyl cytosines, STAR editor-based epigenome editing takes a multiplexing approach with short methylated DNA templates.

In certain embodiments, the use of STAR editor for precise epigenome editing as medicinal agents for treatment of diseases by a hybrid delivery approach consisting of delivering a Cas protein in a tissue tropic AAV and one or more molecule conjugates comprising covalently-joined guide RNA and DNA template (DNA is selectively methylated by design) either naked or in a non-viral vector. Alternatively, the epigenome editing STAR editor is delivered as an RNA mixture packed in a non-viral vector, e.g., LNP nanoparticles.

In some aspects, the invention provides synthetic guide RNA-DNA template conjugates comprising a hypomethylated DNA template.

In some aspects, the invention provides synthetic guide RNA-DNA template conjugates comprising a hypermethylated DNA template.

In some aspects, the invention provides synthetic guide RNA-DNA template conjugates comprising a DNA template which contains one or more 5-methyl cytosines.

In some aspects, the invention provides a hybrid delivery approach consisting of delivering a Cas protein in a tissue tropic AAV and one or more molecule conjugates comprising covalently-joined guide RNA and DNA template (DNA is optionally methylated by design) either naked or in a non-viral vector.

In certain embodiments, lgRNA-oligonucleotide conjugates comprise a guide RNA and an RNA segment for reverse transcription. The RNA segment comprises a reverse transcription template (RTT) and a prime binding sequence (PBS) in 5′→3′ direction.

In certain embodiments, lgRNA-oligonucleotide conjugates comprise an RNA segment for reverse transcription. The RNA template comprises a reverse transcription template (RTT) and a PBS of 7-14 nt in 5′→3′ direction. The PBS hybridizes with the primer which is the PAM distal fragment of the R-loop asymmetrically released from Cas9:lgRNA:DNA complexes or nCas9 (H840A):lgRNA:DNA complexes after nicking to form a complementary duplex of 7-14 nt upstream and before a PAM proximal sequence (6-13 nt including cutting site, 5′→3′). The complementarity between RTT and the PAM proximal sequence (including PAM) is minimized by silent mutations (e.g., replacement with alternative codons).

### DETAILED DESCRIPTION OF THE INVENTION

The most commonly used type of CRISPR system for gene regulation, disruption or correction to date is type II represented predominantly by Cas9. CRISPR-Cas9 is a naturally occurring defense system of bacteria. The CRISPR Cas9 endonuclease is activated by the binding of crRNA:tracrRNA and responds specifically to the DNA sequence (target strand) complementary to the spacer in crRNA and cleaves it upon the recognition of a protospacer adjacent motif (PAM) on the 3′-end of the non-target DNA strand. The presence of tracrRNA and Cas9 is required for processing pre-crRNA into individual crRNA by a double-stranded RNA specific ribonuclease, RNase III, forming crRNA:tracrRNA duplexes. This process excludes any futile secondary structures formed between crRNA and tracrRNA other than the active conformation by repeat:anti-repeat recognition. This duplex was fused into an artificial single molecule guide RNA (sgRNA via nucleotide tetraloop or lgRNA via a non-Nucleotide linker) for genome engineering purpose and other applications. In comparison to a single RNA molecule of sgRNA, lgRNA is a RNA conjugate and has repeat:anti-repeat stem structure and/or stem loop 2 (Nishimasu H. and et al. Cell, 2014, 156, 935-49) locked by nNt-linkers.

Attractive alternatives include other class 2 CRISPR systems such as Type V and VI.

An aspect of the invention is directed to compositions of PEGylated guide RNA(s)-Cas protein complex(es) and their uses as medicinal agents in treatment of viral infectious diseases and as gene regulation, disruption and/or correction based therapeutics. The PEG polymers can be other polymers, ligands for cellular receptors, lipids, oligonucleotides, antibodies, polysaccharides or peptides, conjugated to Cas protein and/or guide RNA(s).

In some embodiments, the crRNAs, sgRNAs and lgRNAs are chemically modified for optimization for better efficiency in cleaving target DNAs such as viral genomic DNAs and for minimizing off-target cleavages of host genomic DNAs with or without engineering Cas proteins.

In some embodiments, the crRNAs, sgRNAs or lgRNAs are chemically modified and conjugated with one or more ssDNA donor templates.

In some embodiments, the conjugate ssDNA donor template comprises one or more 5-methyl cytosines.

In some embodiments, the lgRNA-Cas protein complex conjugates are administrated to edit pathogenic genes, of which the lgRNA or lgRNA conjugate comprises one or more chemically ligated ssDNA donor template including optional chemical modifications.

In some embodiments, the pathogenic genes are episomal and integrated viral DNA.

In some embodiments, the pathogenic genes are mutated host genes or any genes to be edited.

An aspect of the invention is directed to compositions of viral vectors encoding a tracrRNA-Cas protein binary complex, and chemically modified oligonucleotides of crRNAs or crRNA conjugates, both of which are delivered to the same targeted cells, preparation methods of said compositions, and uses as therapeutic agents in treatment of viral infectious diseases and as gene regulation, disruption and/or correction based therapeutics, and a method to deliver a tracrRNA-Cas protein binary complex in a tissue tropic viral vector and chemically modified crRNA(s) or crRNA conjugate with cell targeting ligands either in an aqueous solution gymnotically or with transfection reagents or in a non-viral carrier by co-injections or separate injections.

Another aspect of the invention is directed to compositions of viral vectors encoding a Cas protein, and chemically modified oligonucleotides of lgRNAs, sgRNAs or lgRNA/sgRNA conjugates delivered to the same targeted cells, and a method to deliver a Cas protein in a tissue tropic viral vector and chemically modified lgRNA(s), sgRNAs or lgRNA/sgRNA conjugates with cell targeting ligands either in an aqueous solution gymnotically or with transfection reagents or in a non-viral carrier by co-injections or separate injections, in treatment of viral infectious diseases and as therapeutics based on gene disruption and/or correction.

Yet another aspect of the invention is directed to compositions of viral vectors encoding a Cas protein, which functions in the absence of a tracrRNA, and chemically modified oligonucleotides of crRNAs or crRNA conjugates, delivered to the same targeted cells, and a method to deliver Cas protein in a tissue tropic viral vector and chemically modified crRNA(s) or crRNA conjugates either in an aqueous solution gymnotically or with transfection reagents or in a non-viral carrier by co-injections or separate injections, in treatment of viral infectious diseases and as therapeutics based on gene disruption and/or correction.

Yet another aspect of the invention is directed to compositions of viral vectors encoding a Cas protein lacking NLS in eukaryotic cells, the Cas protein functions in the cells only at the presence of a guide RNA-peptide conjugate and the peptide comprises at least one NLS.

Yet another aspect of the invention is directed to compositions of viral vectors encoding a Cas protein lacking NLS in mammalian cells, the Cas protein functions in the cells only at the presence of a guide RNA-peptide conjugate and the peptide comprises at least one NLS.

Yet another aspect of the invention is directed to administration of a guide RNA-peptide conjugate (the peptide of which comprises at least one NLS) to eukaryotic cells or mammalian cells expressing a Cas protein lacking NLS, and the Cas protein enters nucleus only after complexing with the guide RNA-peptide conjugate.

In some embodiments, the crRNA, sgRNA and lgRNA or their conjugates are chemically modified for optimization for better efficiency in cleaving target DNAs such as viral genomic DNAs and for minimizing off-target cleavages of host genomic DNAs with or without engineering Cas proteins.

In some embodiments, the Cas protein is engineered, or a CRISPR-associated protein of smaller size thus more amenable to viral delivery in human cells, for efficient administrations and better dosage forms.

In other embodiments, the said compositions are administrated, either alone or in combination with small molecule therapeutic agents, therapeutic proteins such as antibodies, or nuclei acids such as mRNAs, antisense oligonucleotides and small interfering RNAs.

### BRIEF DESCRIPTION OF THE DRAWINGS

FIG. 1: Schematic structure of a Cas9/lgRNA complex conjugate.

FIG. 2: Schematic structures of guide RNAs conjugated with tris-GalNAc ligand(s).

FIG. 3: Schematic structure of a PEGylated Cas9/lgRNA complex with guide RNAs conjugated with tris-GalNAc(s).

FIG. 4: Method to prepare a PEGylated Cas9/lgRNA complex by selective conjugations at serine and lysine residues of a preformed RNP complex.

FIG. 5: Method to prepare a PEGylated Cas9/lgRNA complex by selective conjugations at lysine residues of a preformed RNP complex.

FIG. 6: Method to prepare a PEGylated Cas9/lgRNA complex by selective conjugations at cysteine residues of an engineered Cas protein obtained by site-directed mutagenesis, followed by RNP formation.

FIG. 7: Model of a Cas9/lgRNA-ssDNA in synchronizing actions with host enzymes. Host DNA polymerase extends the PAM distal strand of the R-loop asymmetrically released from a Cas9:lgRNA:DNA complex after hybridization with a conjugated ssDNA and uses the ssDNA as the template. The DNA polymerase (domain) and Cas9 protein can be fused as a single fusion protein. The DNA polymerase can alternatively be derived from other organisms other than mammal, is capable of template-dependently extending 3′-end of a primer, and optionally has 5′→3′ or 3′→5′ exonuclease activity or both. The Cas9 protein is replaceable by other single Cas proteins. The Cas9 is optionally partially deactivated as a nickase, such as H840A or D10A of Streptococcus pyogenes Cas9 (SpCas9).

FIG. 8: Multiple-turnover STAR (Seek-Tag-Amend-Release) CRISPR-Cas9 for editing pathogenic genes with enhanced release of the acting RNP complex. 1. Seek: a Cas9/lgRNA-ssDNA complex binds a double-stranded DNA sequence that contains a sequence match to the 17-20 nucleotides of the lgRNA (spacer) and immediately before a protospacer adjacent motif (PAM) to form an R-loop. 2. Tag: HNH cleaves the target strand at the position 3 bases upstream of the PAM, while less precise cleavage and further 3′-end processing by RuvC leave shortened PAM distal strands of various lengths which are asymmetrically released from a Cas9:lgRNA-ssDNA:DNA complex. The released DNA strand acts as a primer and hybridizes with the 3′-homology arm of the conjugated ssDNA, which acts as a template for DNA repair. 3. Amend: the tagged double strand breaks are repaired and the flaps are removed and gaps are ligated by cellular enzymes. 4. Release: Cas9:lgRNA-ssDNA complex is released from the repaired DNA, and is ready for next cycle. The Cas9 protein can be a nickase, e.g. HNH (H840A), and a DNA nick is formed and repaired in a similar way (STAR) to release the dCas9:lgRNA-ssDNA.

FIG. 9: DNA repair through microhomology-mediated end joining (MHEJ) pathway directed by lgRNA-ssDNA in Cas9-based gene editing.

FIG. 10: DNA repair through microhomology-mediated end joining (MHEJ) pathway directed by lgRNA-ssDNA in Cpf1-based gene editing.

FIG. 11: In vitro cleavage data of lgRNA-ssDNA conjugates. ssDNA had a complementary PAM distal sequence of various lengths (primer binding sequence, PBS). The lengths of PBS were 7 nt (lane 2), 9 nt (lane 3), 10 nt (lane 4), 11 nt (lane 5), 12nt (lane 6), 13 nt (lane 7), 14 nt (lane 8) and 17 nt (lane 9). Lane 1 was lgRNA and lane 10 was a negative control. The ssDNA was linked to 5′-or 3′-end or a ligation site of lgRNA at its 3′-or 5′-end. Sec SEQ ID NO: 77 for an example structure of the conjugate. The ssDNA binds to a non-target strand released by CRISPR cleavage, and functions as a template for gene editing by a DNA-directed polymerase.

FIG. 12: Efficiencies of STAR editing in a biochemical assay. The lengths of PBS were 9 nt (lane 1), 10 nt (lane 2) and 11 nt (lane 3). The reaction mixture comprised nCas9 (H840A), DNA polymerase, lgRNA-ssDNA conjugate and synthetic dsDNA substrate in a cleavage buffer.

FIG. 13: Shape complementarity of Cas9-gRNA:DNA duplex recognitions. The major groove between position 8 and 13 (5′→3′ of gRNA) is accessible for modified nucleic acid bases such as a G-clamp.

FIG. 14: Binding of Cas9 at major and minor grooves by hydrogen bonds.

FIG. 15: Example of a bound G-clamp.

FIG. 16: Complementary hydrogen bonding in major and minor grooves of A-Clamps and G-Clamps.

FIG. 17: Positions (N8, N9, N10, N11, N12 and N13) where the nucleotide was replaced with a 2′-deoxy G-Clamp nucleotide (B-8-1). The cleavage activities of corresponding lgRNA-CRISPR-Cas9 complexes are given in FIG. 18.

FIG. 18: G-Clamp modifications of lgRNAs at major groove maintained or increased cleavage activity of lgRNA-CRISPR-Cas9 complexes. See FIG. 17 for the positions (N8, N9, N10, N11, N12 and N13) where a cytidine was replaced with a 2′-deoxy G-Clamp nucleotide (B-8-1). In the example of lgRNA-3, its off-target cleavage was substantially reduced by G-clamp modifications (N10 and N11). A synergetic effect of modifications at more than one position is expected. In addition, 5-methylcytosine is smaller than G-clamp, and is expected to be compatible in this region and can replace cytosine(s) for increasing affinity, specificity and nuclease stability.

FIG. 19: AAV vector encoding a Cas9:tracrRNA complex. The promotors are tissue selective human promotors, which can be inducible, and Cas9 can be a dCas9-effector fusion protein or Cas9 nickase, or any other Cas protein. NLS is optional at the presence of a crRNA-NLS conjugate.

FIG. 20: AAV vector encoding a Cas9 protein. The promotors are tissue selective human promotors, which can be inducible, and Cas9 can be a dCas9-effector fusion protein or Cas9 nickase, or any other Cas protein. NLS is optional at the presence of a lgRNA-NLS or sgRNA-NLS conjugate.

FIG. 21: Division of ternary Cas9:crRNA:tracrRNA RNP complexes into variable crRNA(s) and a fixed binary Cas9:tracrRNA RNP complex for its cellular delivery, and Cas9 can be a dCas9-effector fusion protein or Cas9 nickase, or any other Cas protein. A binary Cas9:tracrRNA RNP complex can be prepared in vitro and delivered to target cells (step b and c) or formed from a Cas9 protein and tacrRNA in target cells (step a). The binary Cas9:tracrRNA RNP complex forms a ternary Cas9:crRNA:tracrRNA RNP complex upon addition of crRNA(s) or its conjugates (step d). Ternary Cas9:crRNA:tracrRNA RNP complexes and their conjugates can also be alternatively prepared in vitro, and are delivered to target cells (step e and f).

FIG. 22: Schematic depiction of administration of an AAV vector encoding both Cas9-NLS and tracrRNA, and crRNA/crRNA conjugates by injections. NLS is optionally absent at the presence of a guide RNA-NLS conjugate.

FIG. 23: Schematic depiction of administration of an AAV vector encoding Cas9-NLS, and lgRNA/lgRNA conjugates by injections. NLS is optionally absent at the presence of a guide RNA-NLS conjugate.

FIG. 24: Schematic depiction of administration of a mixture of mRNA (Cas9-NLS or Cas9) and lgRNA-conjugates packaged in lipid nanoparticles by injections. NLS is optionally absent at the presence of a guide RNA-NLS conjugate.

FIG. 25: Schematic depiction of administration of an AAV vector encoding both Cas9-NLS and tracrRNA, and crRNA/crRNA conjugates by IV injections and the formation of RNP complex conjugates in the liver. NLS is optionally absent at the presence of a guide RNA-NLS conjugate.

FIG. 26: Schematic depiction of administration of a mixture of mRNA (Cas9-NLS or Cas9) and lgRNA/lgRNA-conjugates packaged in lipid nanoparticles by IV injections and the formation of RNP complex conjugates in the liver.

FIG. 27: Schematic depiction of administration of an AAV vector encoding Cas9-NLS, and lgRNA/lgRNA conjugates by IV injections and the formation of RNP complex conjugates in the liver. NLS is optionally absent at the presence of a guide RNA-NLS conjugate.

FIG. 28: Schematic depiction of ex vivo adoptive cell therapy (ACT): allogeneic universal CAR-T cells as an example. NLS is optionally absent at the presence of a guide RNA-NLS conjugate.

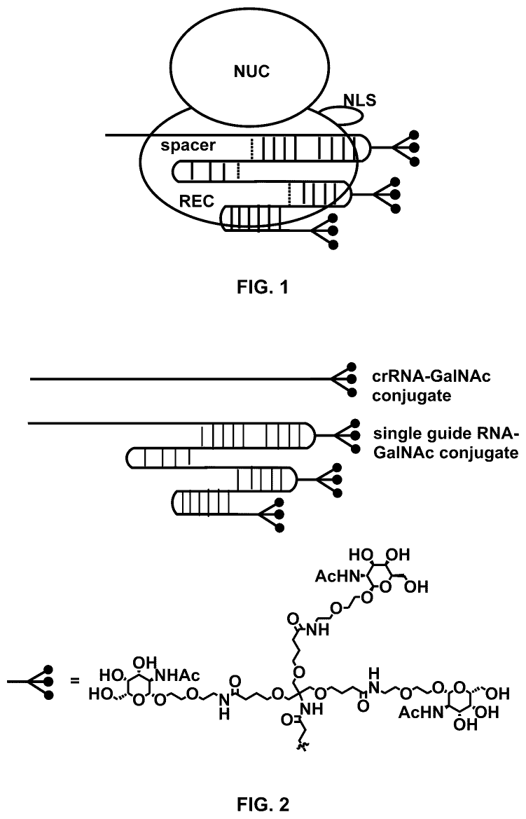

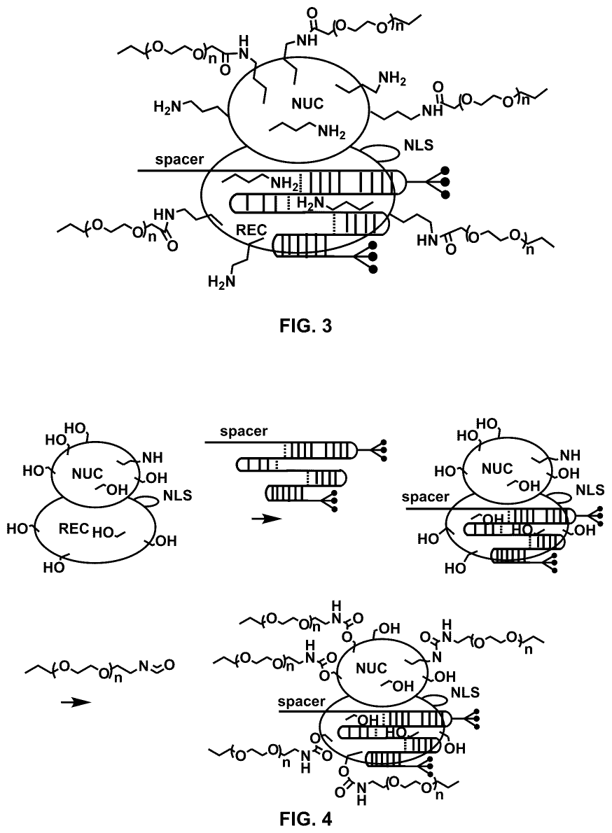

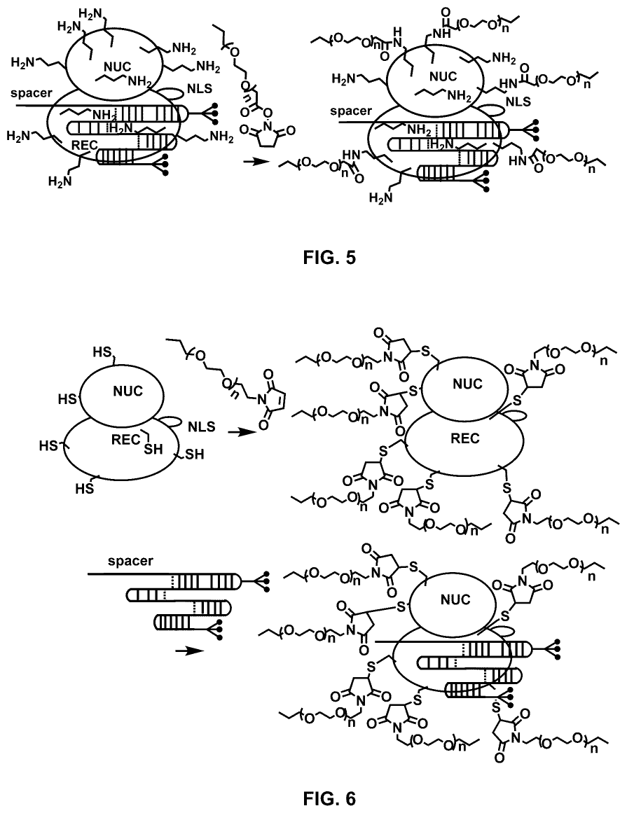

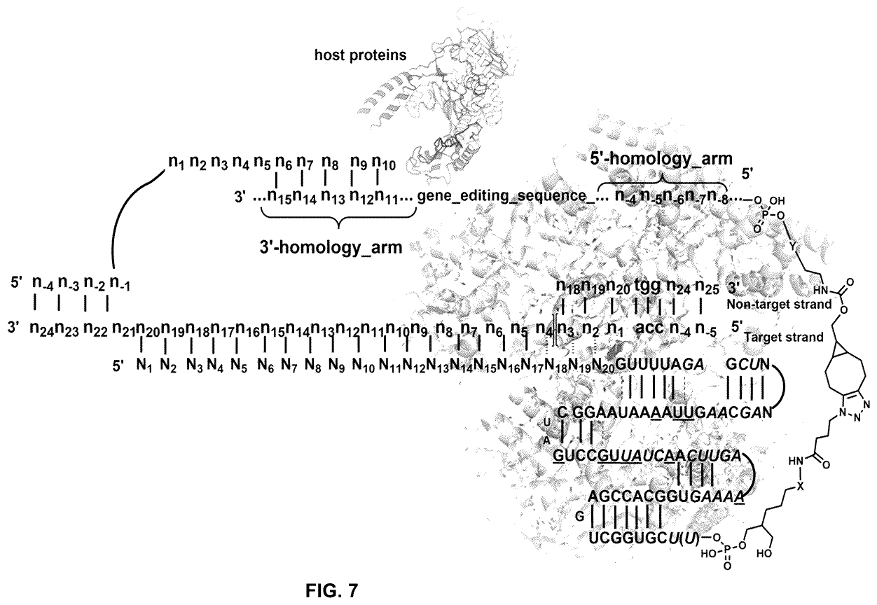

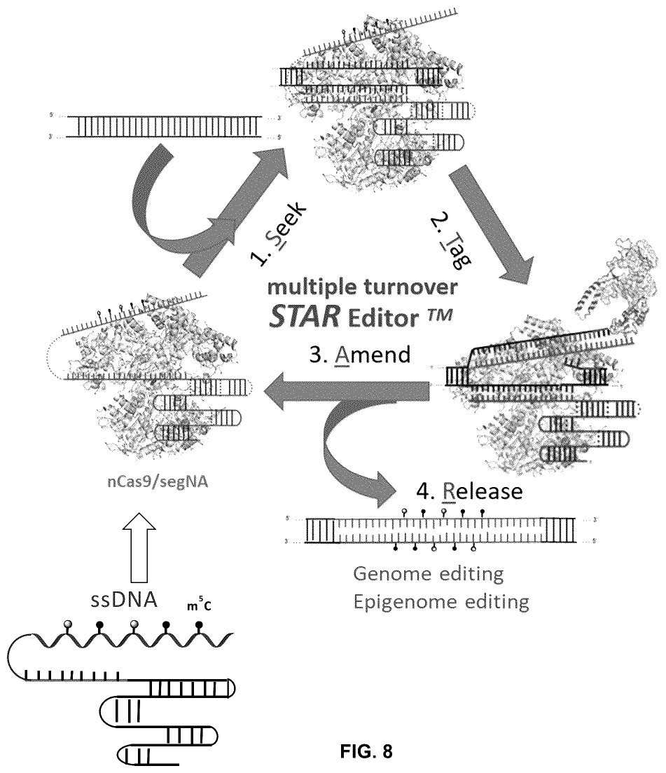

### DEFINITION

The definitions of terms used herein are consistent to those known to those of ordinary skill in the art, and in case of any differences the definitions are used as specified herein instead.

The term “nucleoside” as used herein refers to a molecule composed of a heterocyclic nitrogenous base, containing an N-glycosidic linkage with a sugar, particularly a pentose. An extended term of “nucleoside” as used herein also refers to acyclic nucleosides and carbocyclic nucleosides.

The term “nucleotide” as used herein refers to a molecule composed of a nucleoside monophosphate, di-, or triphosphate containing a phosphate ester at 5′-, 3′-position or both. The phosphate can also be a phosphonate, phosphoramidate, phosphorodiamidate, phosphonoacetate (PACE), thiophosphonoacetate (thioPACE), or phosphoromonothioate.

The term of “oligonucleotide” (ON) is herein used interchangeably with “polynucleotide”, “nucleotide sequence”, and “nucleic acid”, and refers to a polymeric form of nucleotides of any length, either deoxyribonucleotides or ribonucleotides, or analogs thereof. An oligonucleotide may comprise one or more modified nucleotides, which may be imparted before or after assembly of such an oligonucleotide. The sequence of nucleotides may be interrupted by non-nucleotide components.

The term of “CRISPR-Cas system” refers a prokaryotic immune system that confers resistance to foreign genetic elements such as those present within plasmids and phages that provides a form of acquired immunity. RNA harboring the spacer sequence helps Cas (CRISPR-associated) proteins recognize and cut foreign pathogenic DNA. Other RNA-guided Cas proteins cut foreign RNA. CRISPR are found in approximately 50% of sequenced bacterial genomes and nearly 90% of sequenced archaea. The system is being engineered for gene regulation and editing, insertion, disruption and/or correction in eukaryotic cells.

The term of “CRISPR/Cas9” refers to the type II CRISPR-Cas system such as SpCas9 from Streptococcus pyogenes. The type II CRISPR-Cas system comprises protein Cas9 and two noncoding RNAs (crRNA and tracrRNA). These two noncoding RNAs were further fused into one single guide RNA via a tetraloop (sgRNA) and a chemically ligated guide RNA via one or more nNt-Linkers (lgRNA). The Cas9/sgRNA or Cas9/lgRNA complex binds double-stranded DNA sequences that contain a sequence match to the first 17-20 nucleotides of the guide RNA(s) and immediately before a protospacer adjacent motif (PAM). Once bound, two independent nuclease domains (HNH and RuvC) in Cas9 each cleaves one of the DNA strands 3 bases (HNH) or more (RuvC) upstream of the PAM, leaving a DNA double stranded break (DSB).

The term of “Cas protein” refers to a class 2 CRISPR-Cas protein.

The term of “off-target effects” refers to non-targeted cleavage of the genomic DNA target sequence by Cas9 or any other Cas protein despite imperfect matches between the gRNA sequence and the genomic DNA target sequence. Single mismatches of the gRNA can be permissive for off-target cleavage by Cas9. Off-target effects were reported for all the following cases: (a) same length but with 1-5 base mismatches; (b) off-target site in target genomic DNA has one or more bases missing (‘deletions’); (c) off-target site in target genomic DNA has one or more extra bases (‘insertions’).

The term of “guide RNA” (gRNA) refers to the RNA component of a CRISPR-Cas system, e.g. crRNA, dual guide RNAs, a synthetic fusion of crRNA and tracrRNA via a tetraloop (GAAA) (defined as sgRNA) or other chemical linkers such as an nNt-Linker (defined as lgRNA), which is used interchangeably with “chimeric RNA”, “chimeric guide RNA”, “single guide RNA” and “synthetic guide RNA”. The gRNA of CRISPR-Cas9 contains secondary structures of the repeat:anti-repeat duplex, stem loops 1-3, and the linker between stem loops 1 and 2.

The term of “dual RNAs” or “dual guide RNAs” refers to a hybridized complex of the short CRISPR RNAs (crRNA) and the trans-activating crRNA (tracrRNA). The crRNA hybridizes with the tracrRNA to form a crRNA:tracrRNA duplex, which is loaded onto a Cas protein to direct the cleavage of cognate DNA sequences bearing appropriate protospacer-adjacent motifs (PAM).

The term of “lgRNA” refers to a guide RNA (gRNA) joined by chemical ligations to form non-nucleotide linkers (nNt-linkers) between a crgRNA and a tracrgRNA, or at other sites.

The terms of “dual lgRNA”, “triple lgRNA” and “multiple lgRNA” refer to hybridized complexes of the synthetic guide RNA fused by chemical ligations via non-nucleotide linkers. A dual tracrgRNA is formed by chemical ligation between a tracrgRNA1 and a tracrgRNA2 (RNA segments of ˜30 nt), and a crgRNA (˜30 nt) is fused with a dual tracrgRNA to form a triple lgRNA duplex, which is loaded onto Cas9 to direct the cleavage of cognate DNA sequences bearing appropriate protospacer-adjacent motifs (PAM). Each RNA segment can be readily accessible by chemical manufacturing and compatible to extensive chemical modifications.

The term “guide sequence” refers to the about 20 bp sequence within the guide RNA that specifies the target site and is herein used interchangeably with the terms “guide” or “spacer”. The term “tracr mate sequence” may also be used interchangeably with the term “direct repeat(s)”.

The term of “crgRNA” refers to a crRNA equipped with chemical functions for conjugation/ligation. The oligonucleotide may be chemically modified close to its 3′-end, any one or several nucleotides, or for its full sequence. A crgRNA may also be prepared by in vitro transcription at the presence of a RNA polymerase such as bacteriophage T7 RNA polymerase, and the conjugating chemical function, e.g., amine and alkyne, is incorporated at its 5′-end (preferably as 5′-GU . . . or 5′-GC . . . primers with modifications), and 3′-end from a nucleoside triphosphate analogue, e.g. CTP and UTP:

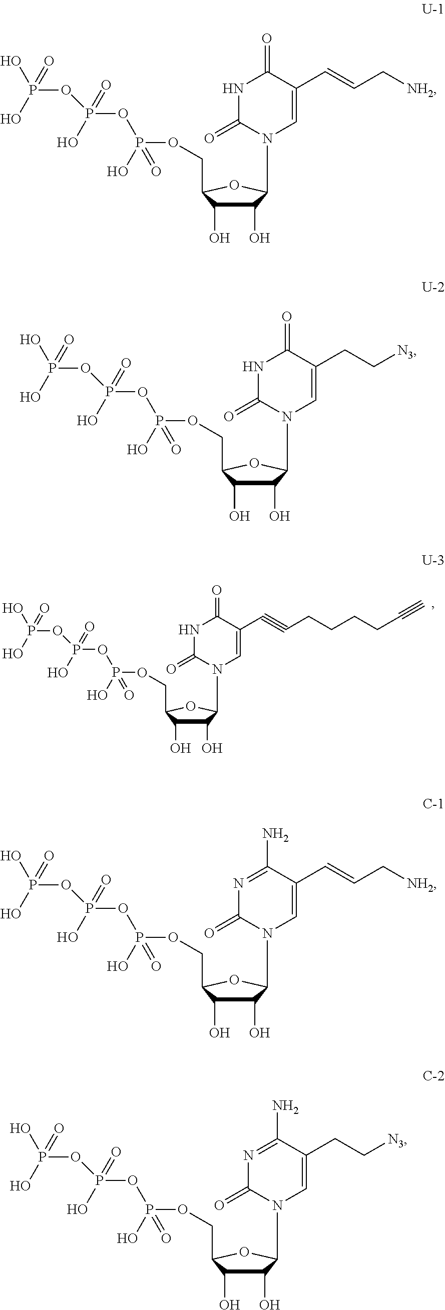

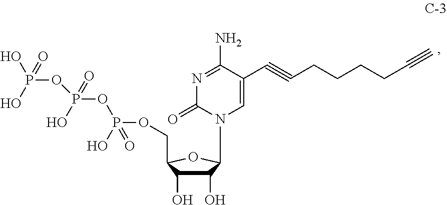

and etc.

The term of “tracrgRNA” refers to a tracrRNA equipped with chemical functions for conjugation/ligation. The oligonucleotide may be chemically modified at any one or several nucleotides, or for its full sequence by chemical synthesis. A tracrgRNA may also be prepared by in vitro transcription at the presence of a RNA polymerase such as bacteriophage T7 RNA polymerase, and the conjugating chemical function, e.g., amine and alkyne, is incorporated at its 5′-end (preferably as 5′-GU . . . or 5′-GC . . . primers with modifications), and 3′-end from a nucleoside triphosphate analogue, e.g. CTP and UTP.

The term of “the protospacer adjacent motif (PAM)” refers to a DNA sequence immediately following the DNA sequence targeted by Cas9 in the CRISPR bacterial adaptive immune system, including NGG, NNNNGATT, NNAGAA, NAAAC, and others from different bacterial species where N is any nucleotide. In CRISPR-Cas12a system, “PAM” refers to a DNA sequence such as TTTN immediately before the targeted DNA sequence.

The term of “chemical ligation” refers to joining together synthetic oligonucleotides via an nNt-linker by chemical methods such as click ligation (the azide-alkyne reaction to produce a triazole linkage), thiol-maleimide reaction, and formations of other chemical groups.

The term of “complementary” refers to the ability of a nucleic acid to form hydrogen bond(s) with another nucleic acid sequence by either traditional Watson-Crick or other non-traditional types. Cas9 contains two nuclease domains, HNH and RuvC, which cleave the DNA strands that are complementary and non-complementary to the 20 nucleotide (nt) guide sequence in crRNAs, respectively.

The term of “a donor template” refers to a transgene cassette or a gene-editing-sequence flanked with homologous regions to recombine with the host loci and replace the mutated DNA with the correct sequence by HDR/SSTR. A donor template can be an ssDNA or a dsDNA or a plasmid/vector, and may be chemically conjugated to guide RNA(s) or Cas protein via a covalent linker.

A donor template can be chemically synthesized and equipped with chemical functions for conjugations/ligations. A conjugating donor template may also be prepared by in vitro gene synthesis at the presence of a DNA polymerase, with chemical functions, e.g. an amine and an alkyne, enzymatically incorporated at its 5′ or 3′-end for chemical conjugation/ligation from a nucleoside triphosphate analogue.

The term of “gene editing sequence”, “gene-editing-sequence” or “gene_editing_sequence” refers to the sequence contained in a donor template sequence to introduce expected gene editing and/or epigenome editing, between the two homology arms identical to the DNA fragments flanking the cleavage site.

The term of “silent mutations” refers to mutations in DNA that do not have an observable effect on the organism's phenotype. Such mutations include a mutation leading to another degenerate codon or a mutation that causes the altered codon to produce an amino acid with similar functionality and thus does not significantly affect protein function.

The term of “Hybridization” refers to a reaction in which one or more polynucleotides form a complex that is stabilized via hydrogen bonding between the bases of the nucleotide residues. The complex may comprise two strands forming a duplex structure, three or more strands forming a multi stranded complex, a single self-hybridizing strand, or any combination of these. A sequence capable of hybridizing with a given sequence is referred to as the “complement” of the given sequence.

The synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include but are not limited to the 2-tetrahydropyranyl group, 2-(bisacetoxyethoxy) methyl group, trityl group, trichloroacetyl group, carbonate-type blocking groups such as benzyloxycarbonyl, trialkylsilyl groups, examples of such being trimethylsilyl, tert-butyldimethylsilyl, tert-butyldiphenylsilyl, phenyldimethylsilyl, triiospropylsilyl and thexyldimethylsilyl, ester groups, examples of such being formyl, (C1-C10) alkanoyl optionally mono-, di- or tri-substituted with (C1-C6) alkyl, (C1-C6) alkoxy, halo, aryl, aryloxy or haloaryloxy, the aroyl group including optionally mono-, di-or tri-substituted on the ring carbons with halo, (C1-C6) alkyl, (C1-C6) alkoxy wherein aryl is phenyl, 2-furyl, carbonates, sulfonates, and ethers such as benzyl, p-methoxybenzyl, methoxymethyl, 2-ethoxyethyl group, etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and G. M. Wuts, T. W. Greene, “Protective Groups in Organic Synthesis”, John Wiley & Sons Inc., Hoboken, New Jersey, 2007, which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include but are not limited to acetyl (Ac), trifluoroacetyl, Boc, Cbz, benzoyl (Bz), N,N-dimethylformamidine (DMF), trityl, and benzyl (Bn). See also G. M. Wuts, T. W. Greene, “Protective Groups in Organic Synthesis”, John Wiley & Sons Inc., Hoboken, New Jersey, 2007, and related publications.

The term “conjugation”, as used herein, refers to a method for covalently crosslinking drug molecules, proteins or nucleic acids to other molecules using crosslinking reagents. The product of conjugation is referred as “conjugate(s)”. Traditional pharmaceuticals can be linked to monoclonal antibodies to deliver targeted doses, prevent breakdown, decrease immunogenicity, and increase bioavailability in circulation. CRISPR RNP complex can be chemically modified by linking to other molecules either covalently or non-covalently.

The term “conjugating site”, as used herein, refers to a chemical moiety which is directly linked to other molecules by conjugation, and a conjugating site can be an amino acid residue, N-terminus or C-terminus of proteins, a nucleoside, a nucleotide, or a phosphate.

The term “PEG,” or “macrogol”, as used herein, refers to polyethylene glycol chains, linear, branched, substituted or unsubstituted. A derivatized linear single PEG chain comprises at least 2 PEG subunits.

The term “peptide” or “peptides”, as used herein, refers to peptide chains, substituted or unsubstituted. A long peptide chain can form a tertiary structure (structural motif), and thus is a protein domain or a protein, such as a cell receptor or antigen binding site or an enzyme domain. A short peptide chain can have a cell penetrating function (CPP) or be a nuclear localization sequence (NLS) or both.

The term “PEGylation”, as used herein, refers to the process of both covalent and non-covalent attachment or amalgamation of polyethylene glycol (PEG) polymer chains to molecules and macrostructures, such as a drug, a CRISPR RNP complex, a therapeutic protein or vesicle, which is then described as PEGylated. PEGylation is routinely achieved by incubation of a reactive derivative of PEG with the target molecule.

The term “glycan”, as used herein, refers to polysaccharides or the carbohydrate portion of a glycoconjugate, such as a glycoprotein, glycolipid, or a proteoglycan, even if the carbohydrate is only an oligosaccharide.

The term “polysaccharides”, as used herein, refers compounds consisting of a large number of monosaccharides linked glycosidically.

The term “epitope” or “antigenic determinant”, as used herein, refers to the part of an antigen that is recognized by the immune system, specifically by antibodies, B cells, or T cells. An epitope can be either conformational or linear.

The term “epitope masking”, as used herein, refers to identifying potentially immunogenic peptide sequences and modifying or removing them to prevent detection by the immune system while still maintaining the therapeutic function of the original protein.

The term of “Isotopically enriched” refers to a compound containing at least one atom having an isotopic composition other than the natural isotopic composition of that atom.

The term of “Isotopic composition” refers to the amount of each isotope present for a given atom, and “natural isotopic composition” refers to the naturally occurring isotopic composition or abundance for a given atom. As used herein, an isotopically enriched compound optionally contains deuterium, carbon-13, nitrogen-15, and/or oxygen-18 at amounts other than their natural isotopic compositions. Conjugates of CRISPR RNP complexes are optionally isotopically enriched at selected positions to optimize their drug properties based on isotope effects.

The term of “Epigenome” refers to inheritable chemical changes to the DNA and histone proteins of an organism, and this invention is particularly related to DNA methylation. The epigenome plays critical in biological processes, such gene expression, development, tissue differentiation, and suppression of transposable elements.

The term of “Epigenome editing” refers to modifying specific sites or region of epigenome using modular modifiers, which comprises a DNA binding protein (DBP) and one or more effector domains (ED). The DBP can be zinc finger protein(s), transcription activator-like effectors (TALE), or dCas proteins such as dCas9. ED can be any chromatin modifying proteins. These modifications include DNA methylation and demethylations, histone methylation, demethylations, acetylation and deacetylation, and others.

The term of “DNA methylation” refers to one or more nucleic acid bases of DNA are methylated. DNA methylations often occur at CpG sites, and play pivotal roles in multiple biological processes such as gene silencing, genomic imprinting and X-chromosome inactivation. Aberrant DNA methylation has been reported in various diseases such as fragile X syndrome (FXS), the immunodeficiency, centromeric instability, and facile anomalies (ICF), and cancer.

The term of “STAR editing” refers to a CRISPR-Cas editor comprising a STAR editing nucleic acid (segNA or segRNA), which comprises a guide RNA and a DNA template tethered to the guide RNA, and a Cas protein optionally fused with a DNA-directed DNA polymerase. STAR (Seek-Tag-Amend-Release) editor functions as follow: 1. Seek: a Cas9/lgRNA-ssDNA complex binds a double-stranded DNA sequence that contains a sequence match to the first 17-20 nucleotides of the lgRNA and immediately before a protospacer adjacent motif (PAM) to form a R-loop. 2. Tag: HNH cleaves the target strand at the position 3 bases upstream of the PAM, while less precise cleavage and further 3′-end processing by RuvC leave shortened PAM distal strands of various lengths which are asymmetrically released from a Cas9:lgRNA-ssDNA:DNA complex. The released DNA strand acts as a primer and hybridizes with the 3′-homology arm of the conjugated ssDNA, which acts as a template for DNA repair. 3. Amend: the tagged double strand breaks are repaired and the flaps are removed and gaps are ligated by cellular enzymes. 4. Release: Cas9:lgRNA-ssDNA complex is released from the repaired DNA, and is ready for next cycle. The Cas protein can be a nickase nCas9, e.g. HNH (H840A), and a DNA nick is formed and repaired in a similar way (STAR) to release the dCas9:lgRNA-ssDNA. STAR editor is capable of full-spectrum genome editing and epigenome editing at a single-nucleotide resolution level. Epigenome editing by a STAR editor unitizes a DNA template with one or more preinstalled methylated nucleic acid bases.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent known to be useful for, or which has been or is currently being used for the treatment or prevention of a disorder or one or more symptoms thereof.

The term of “gene therapy” refers to altering a disease-causing gene in a patient or introducing a healthy copy of a mutated gene to a patient to treat genetic diseases. CRISPR/Cas can potentially be used to introduce site specific gene editing to correct disease-causing mutations, or to deliver a correct gene into human genome to fix a defect gene or a desired gene. CRISPR/Cas can potentially be used to remove and/or deactivate episomal HBV cccDNA and integrated viral genomes such as HIV proviral DNA and integrated HBV DNA to cure these infectious diseases.

It is noted that as used, the singular forms “a,” “an,” and “the” include plural referents unless the context clearly dictates otherwise. Thus, for example, reference to “a guide RNA(s)-Cas protein (RNP) complex” includes a plurality of such complexes. Reference to “the conjugate” includes reference to one or more conjugates and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

### Nucleotides

In some embodiments, the crRNA and tracrRNA are truncated at 3′-end and 5′-end, respectively:

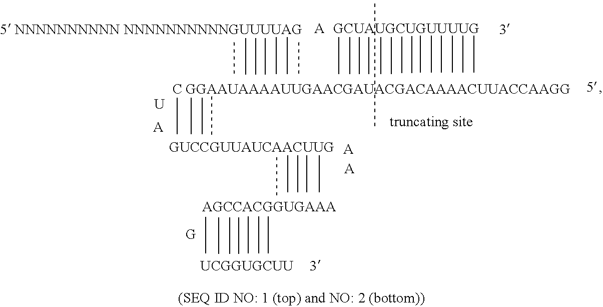

and the duplex ends are rejoined by a small molecule non-nucleotide linker (nNt-linker, ligation1) to form a dual lgRNA:

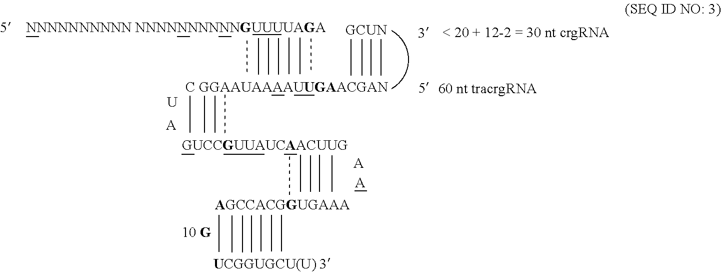

wherein “NNNNNNNNNN NNNNNNNNNN” is a guide sequence of 17-20 nt, and N is preferably a ribonucleotide with intact 2′-OH, and wherein “) [symbol] ” is a chemical nNt-linker.

In some embodiments, the duplex ends are rejoined by a tetraloop (e.g. GAAA) to form an sgRNA:

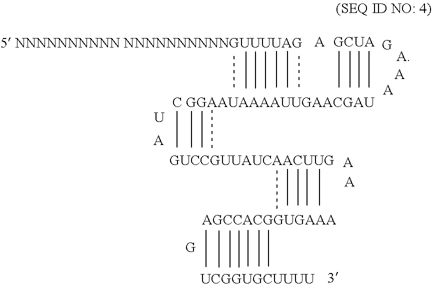

In other embodiments, tracrRNA is a ligated dual oligonucleotide (via ligation2, the inner ligation between tracrgRNA1 and tracrgRNA2), or a multiple oligonucleotide. Non-limiting examples include:

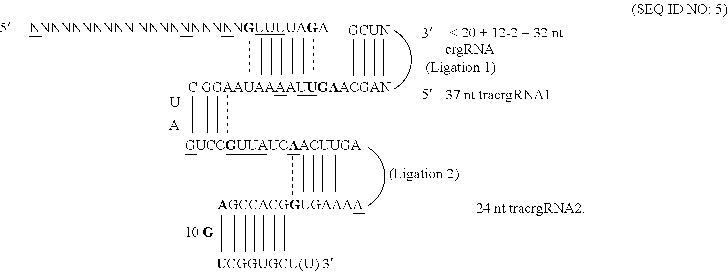

In some embodiments, the crRNA and tracrRNA are further truncated and the ligation site can be located at other positions, as illustrated by non-limiting examples of resulting lgRNAs in U.S. Pat. No. 10,059,940 (cited herein and incorporated by reference in its entirety).

In some embodiments, crRNA and tracrRNA are truncated at 3′-end and 5′-end, respectively, and the duplex ends are rejoined by a nucleotide linker such as an aptamer and thus to provide an extended single gRNA with small molecule and protein recognition module(s):

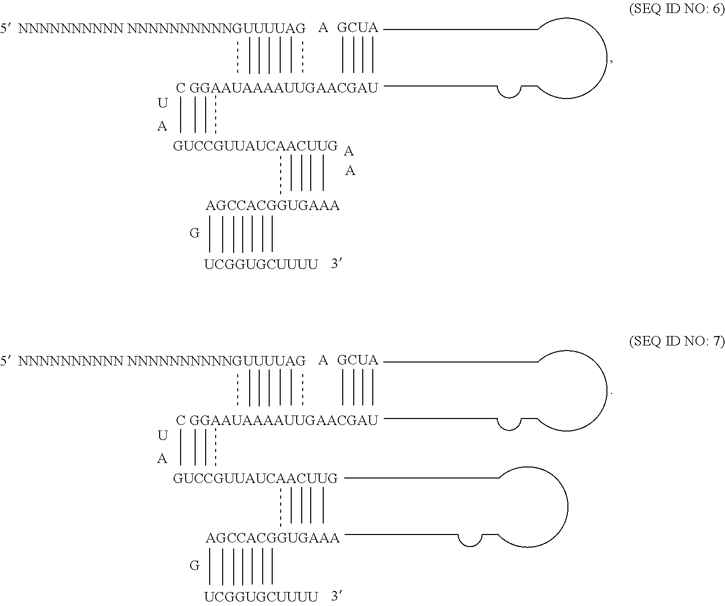

In another embodiment, the crRNA and tracrRNA are truncated at 3′-end and 5′-end, respectively, and the duplex ends are rejoined by a non-nucleotide linker-aptamer conjugate to provide an extended single lgRNA with small molecule and protein recognition module(s):

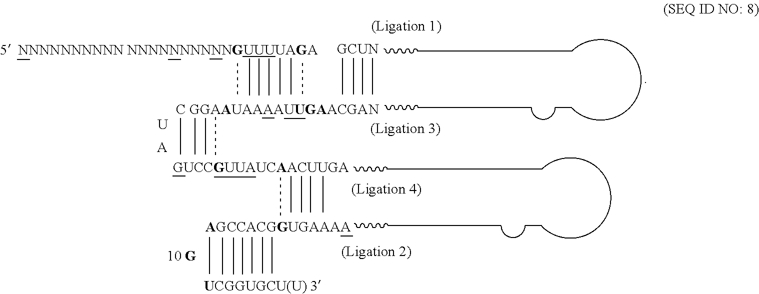

In another embodiment, the stem loop of tracrRNA is split at the GAAA tetraloop, and the duplex ends are rejoined by a non-nucleotide linker-aptamer conjugate to provide an extended tracrRNA with small molecule and protein recognition module(s):

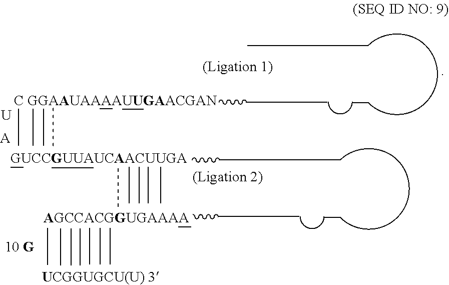

In yet another embodiment, lgRNA is conjugated with an aptamer by a non-nucleotide linker at either of the two GAAA tetraloops or both, or 5′/3′-end of sgRNA, to bind a small molecule or a biopolymer such as a protein or a nucleic acid:

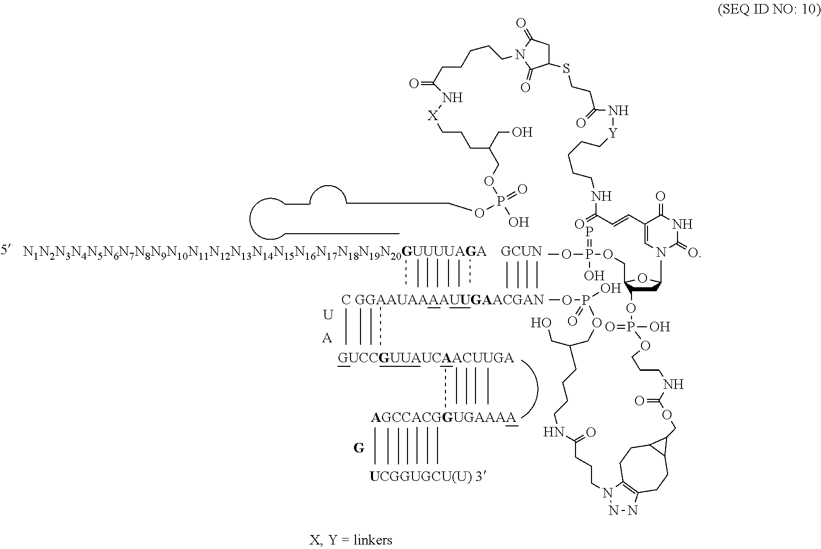

In some embodiments, the crRNA and tracrRNA are shortened by truncation at 3′-end and 5′-end, respectively, and the repeat:anti-repeat duplex comprises a bulge and >12 Watson-Crick base pairs:

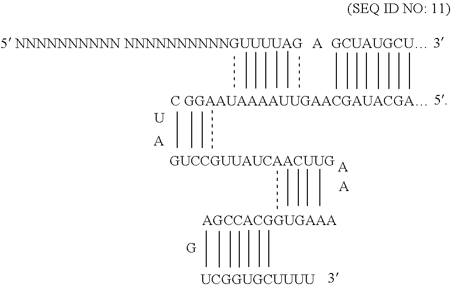

In some embodiments, the crRNA and tracrRNA are joined at 3′-end of tracrRNA and 5′-end of crRNA by a nucleotide linker or a non-nucleotide linker to form a sgRNA or lgRNA, respectively; and the tracrRNA is optionally a ligated tracrRNA comprising one or more than one non-nucleotide linker:

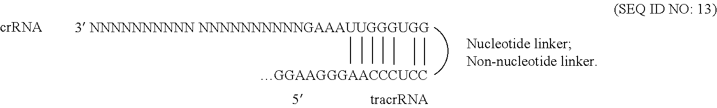

In some embodiments, the dual guide RNAs, sgRNA and lgRNA are conjugates of small molecule ligands, cell penetrating peptides (CPP), nuclear localization signal (NLS) peptides, antibodies or aptamers:

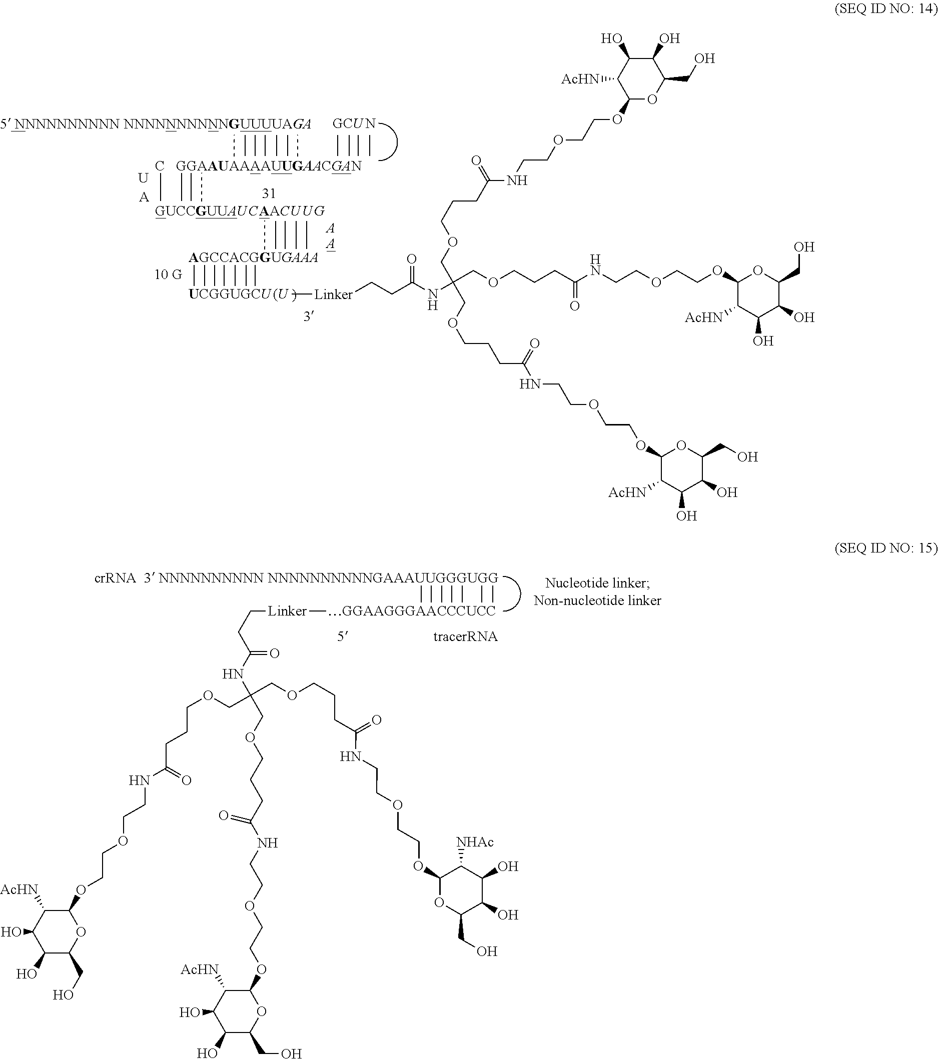

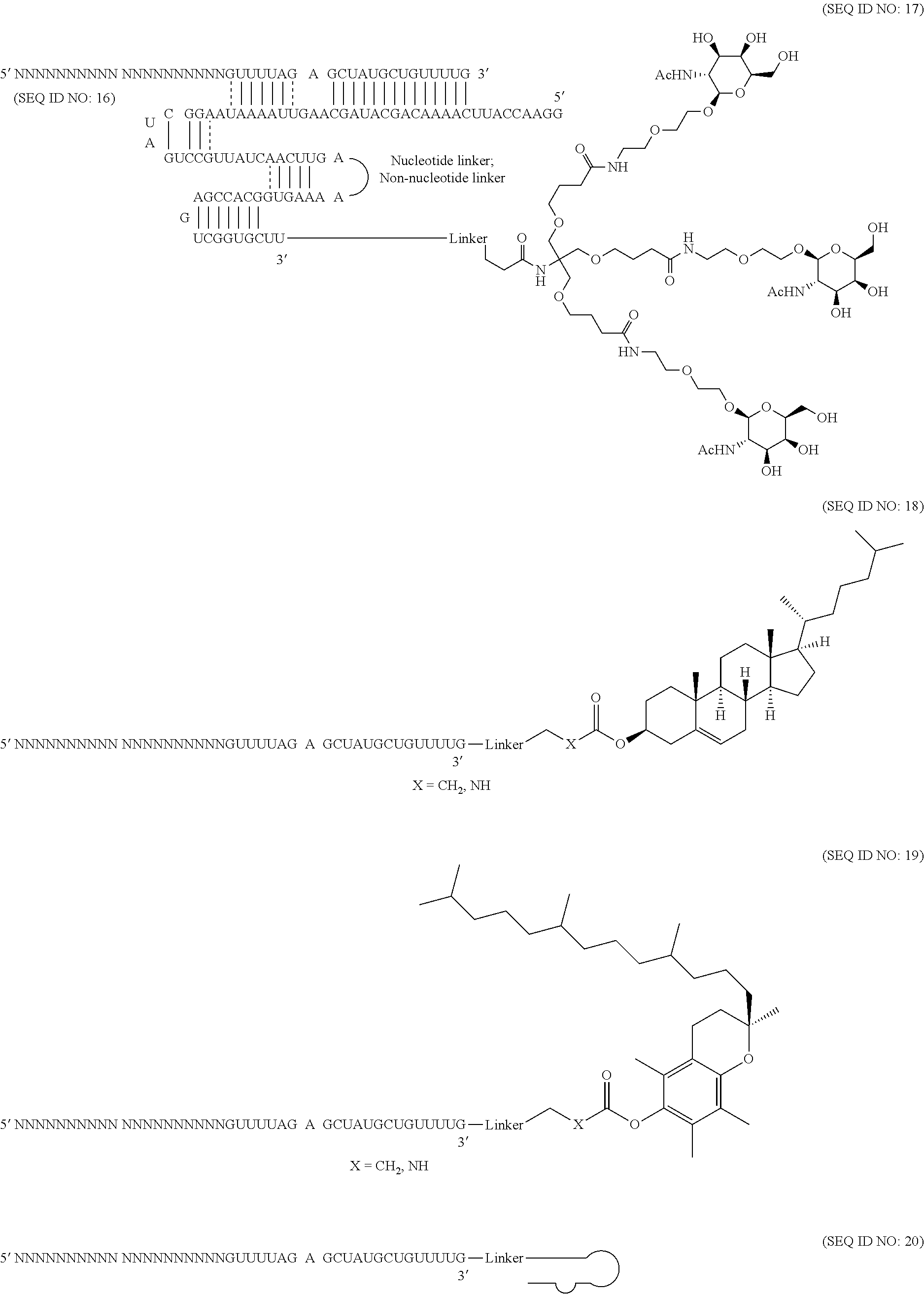

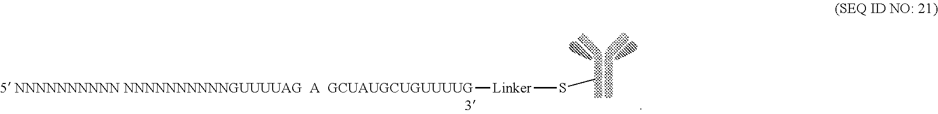

In some embodiments, the crRNA, dual guide RNAs, sgRNA and lgRNA are conjugated with single-stranded template repair (SSTR) donor DNA templates of sequences containing a 5′-homology arm and a downstream gene editing sequence, optionally followed by a 3′-homology arm. Non-limiting such examples are given below:

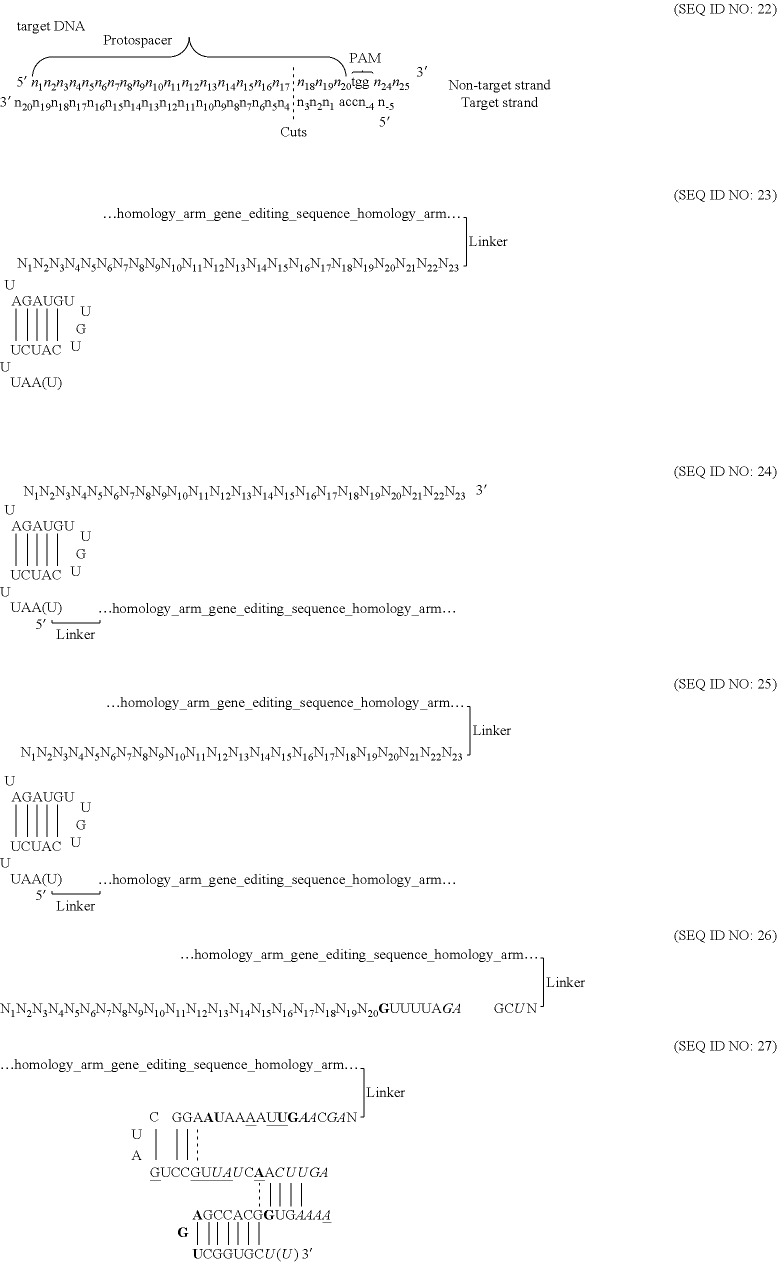

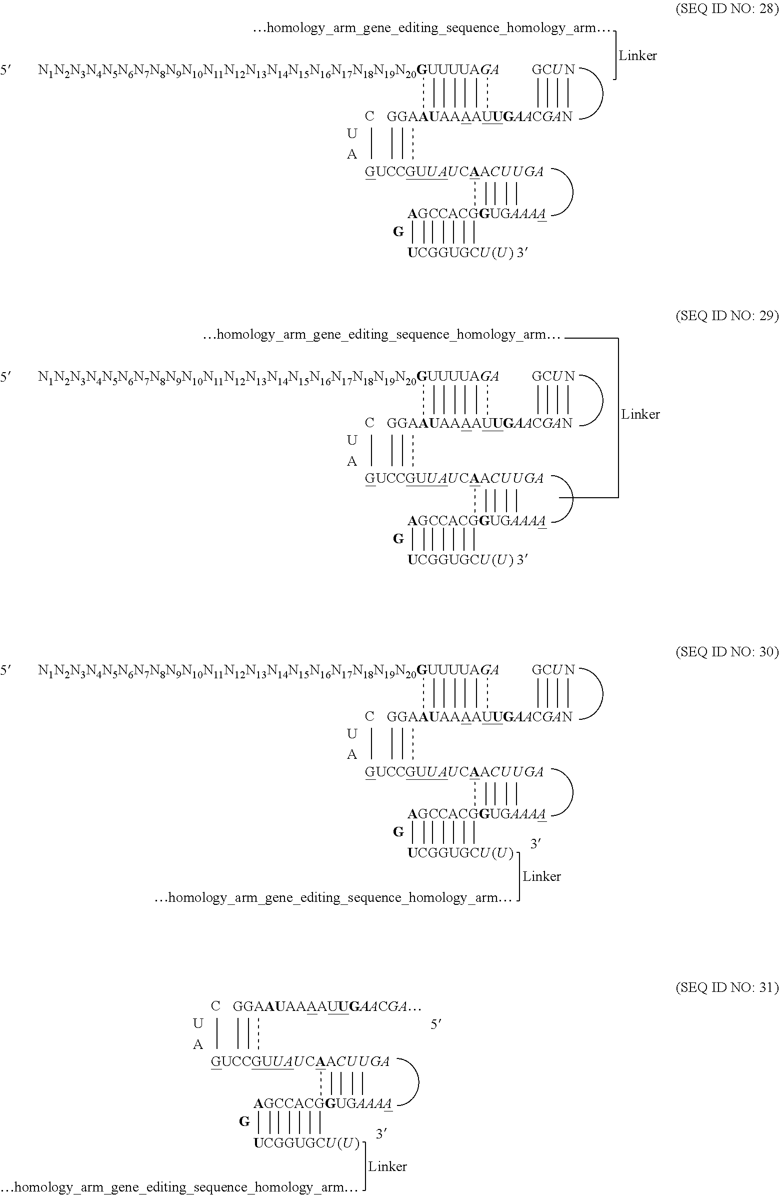

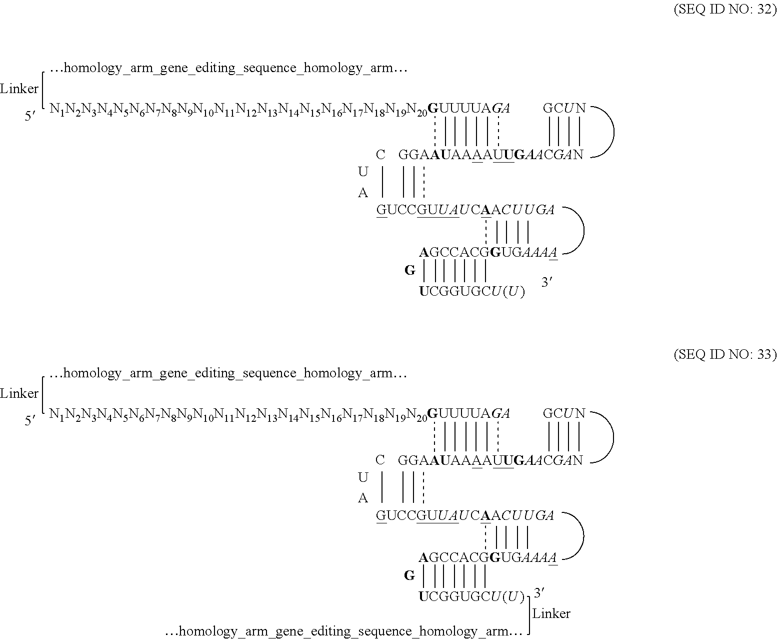

In certain embodiments, the conjugating linker “]” and “ [symbol] ” is an RNA tetraloop such as ANYA, CUYG, GNRA, UNAC and UNCG, wherein N could be uracil, adenine, cytosine, or guanine, R is cither guanine or adenine and Y is either uracil or cytosine.

In certain embodiments, the conjugating linker “]” and “ [symbol] ” is an nNt-linker, as described in U.S. Pat. No. 10,059,940, selected from a group comprising a 1,4-disubstituted 1,2,3-triazole formed between an alkyne and an azide via [3+2] cycloaddition catalyzed by Cu(I) or without Cu(I) catalysis (such as strain-promoted azide-alkyne cycloaddition (SPAAC)), a thioether by chemical ligation between a thiol and a maleimide, and other). functional groups, and “]” and “ [symbol] ” can be the same or different.

In certain embodiments, the conjugating linker “]” and “ [symbol] ” is either a nucleotide linker such as an RNA tetraloop or an nNt-linker.

In certain embodiments, homology arms flanking the gene editing sequence overlap with the non-target PAM containing strand of target DNA.

In certain embodiments, homology arms flanking the gene editing sequence overlap with the target strand of target DNA.

In some embodiments, the conjugating site is the 5-position of the nucleoside base U or C, or the 7-position of the nucleoside base 7-deazaguanine or 7-deazaadenine, or the 8-position of the nucleoside base purine.

In other embodiments, the conjugating site is the 2′- or 5′-position of the sugar moiety of the 5′-end nucleotide of crRNA, tracrRNA, a single strand HDR/SSTR donor template (ssDNA), sgRNA or lgRNA.

In other embodiments, the conjugating site is the 2′- or 3′-position of the sugar moiety of the 3′-end nucleotide of crRNA, tracrRNA, a single strand HDR/SSTR donor template (ssDNA), sgRNA or lgRNA.

In certain embodiments, as non-limiting examples, the lgRNA-ssDNA conjugates comprise the following structures:

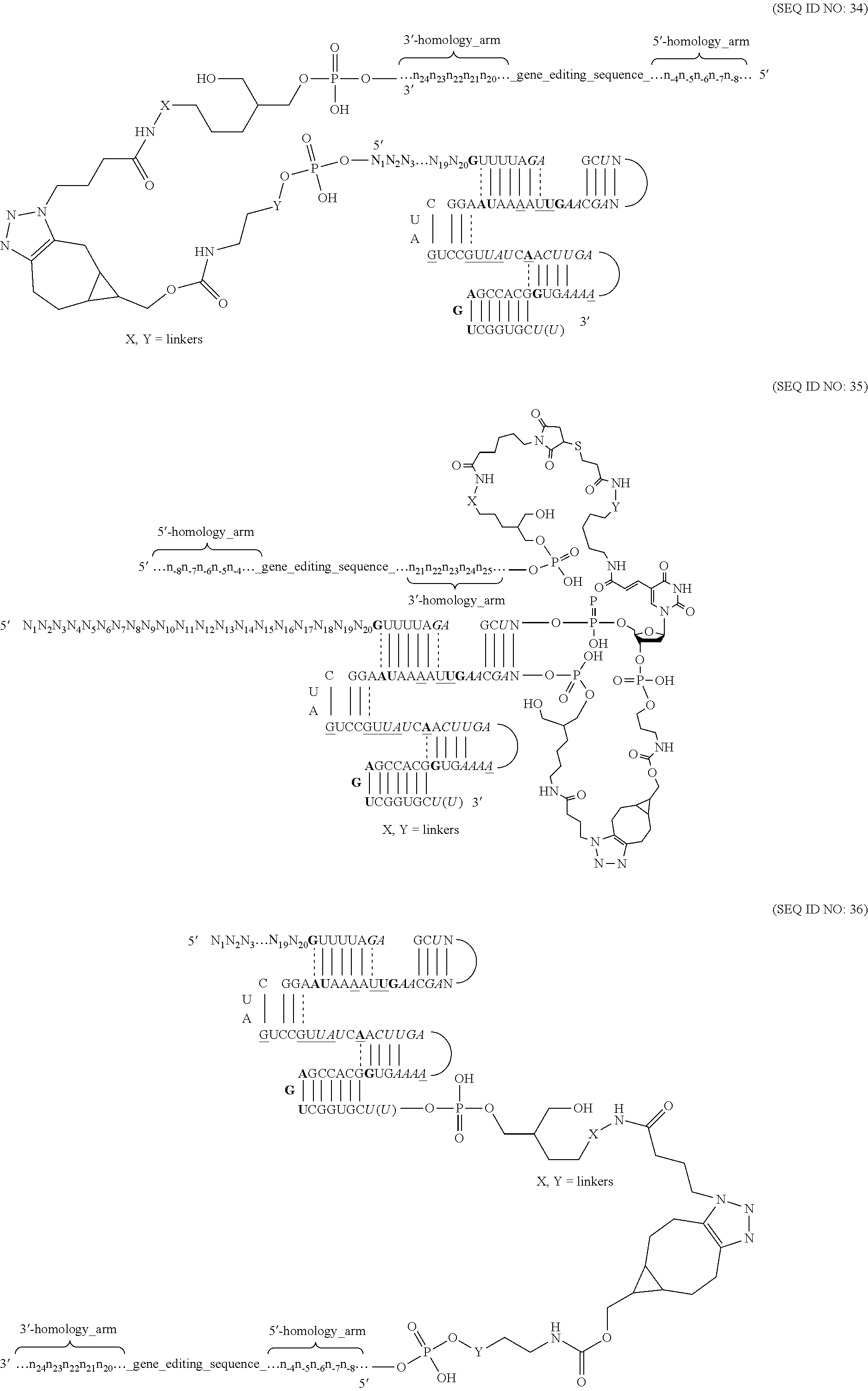

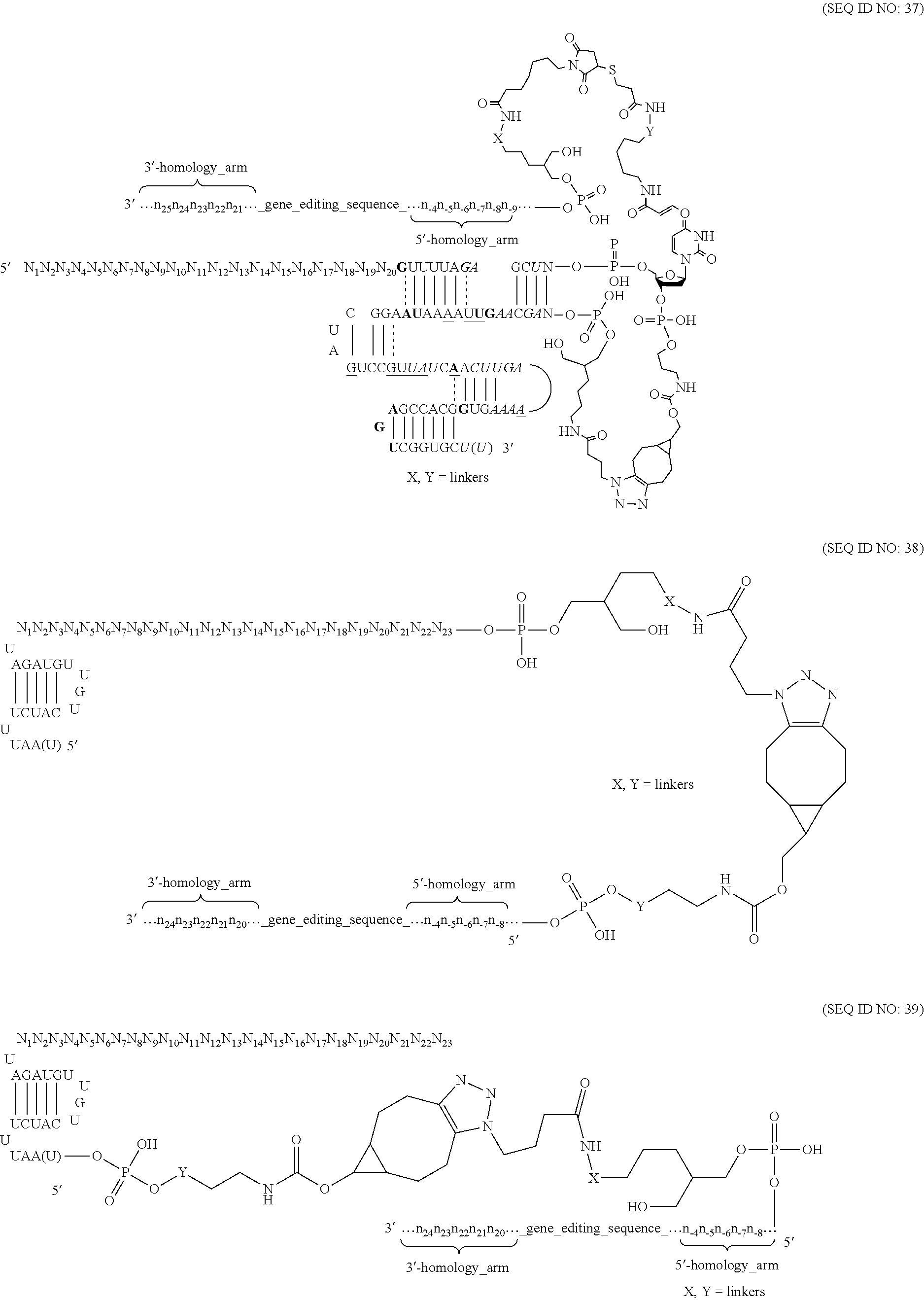

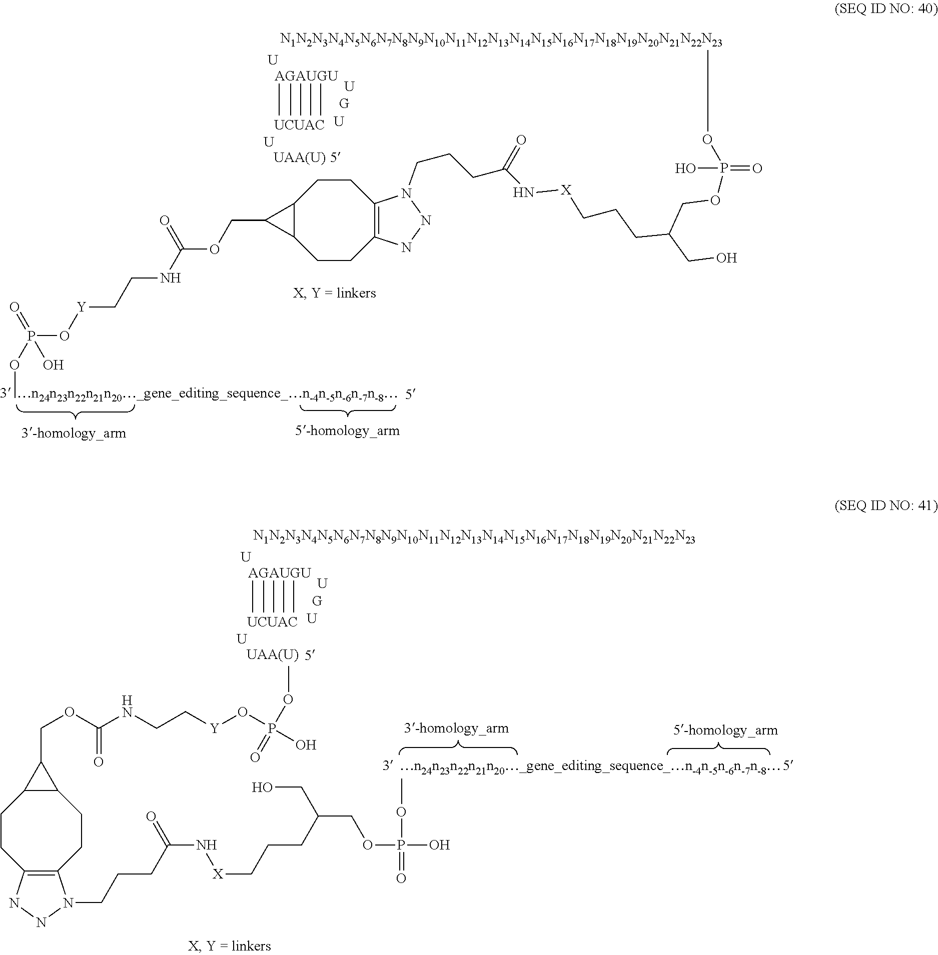

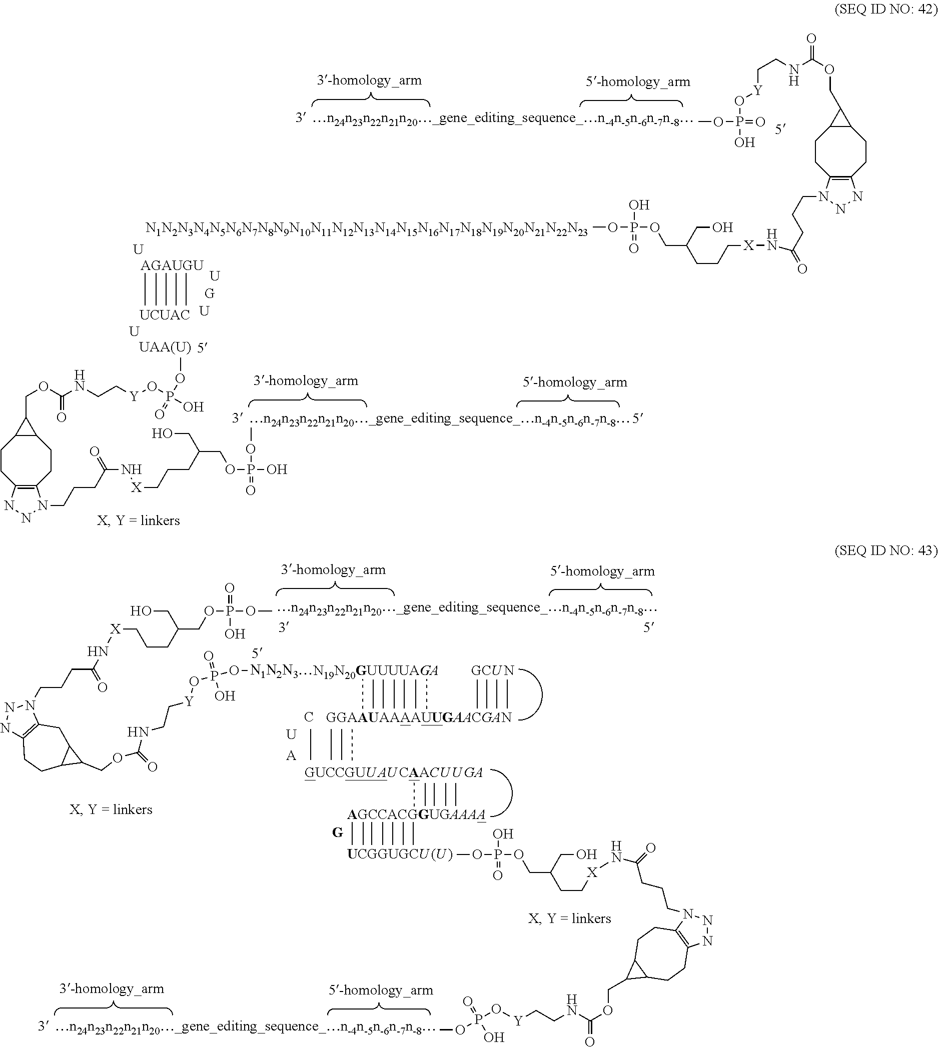

In some embodiments, the “gene-editing-sequence” comprises an oligonucleotide sequence to introduce insertion(s) of one or more stop codons selected from a group comprising 5′-(tga)-3′. 5′-(taa)-3′. 5′-(tag)-3′. 5′-(tga-ntga-ntga)-3′. 5′-(tga-ntga-ntaa)-3′. 5′-(tga-ntga-ntag)-3′, 5′-(tga-ntaa-ntga)-3′, 5′-(tga-ntaa-ntaa)-3′, 5′-(tga-ntaa-ntag)-3′, 5′-(tga-ntga-ntga)-3′, 5′-(tga-ntga-ntaa)-3′, 5′-(tga-ntga-ntag)-3′, 5′-(taa-ntga-ntga)-3′, 5′-(taa-ntga-ntaa)-3′, 5′-(taa-ntga-ntag)-3′, 5′-(taa-ntaa-ntga)-3′, 5′-(taa-ntaa-ntaa)-3′, 5′-(taa-ntaa-ntag)-3′, 5′-(taa-ntga-ntga)-3′, 5′-(taa-ntga-ntaa)-3′, 5′-(taa-ntga-ntag)-3′, 5′-(tag-ntga-ntga)-3′, 5′-(tag-ntga-ntaa)-3′, 5′-(tag-ntga-ntag)-3′, 5′-(tag-ntaa-ntga)-3′, 5′-(tag-ntaa-ntaa)-3′, 5′-(tag-ntaa-ntag)-3′, 5′-(tag-ntga-ntga)-3′, 5′-(tag-ntga-ntaa)-3′ and 5′-(tag-ntga-ntag)-3′, wherein n is any nucleotide, and said more stop codons comprise repetitive said sequence separated by absent or more nucleotides in between or different said sequences separated by absent or more nucleotides in between.

In some embodiments, the “gene-editing-sequence” comprises one or more sequences such as stop codons to deactivate integrated and episomal viral DNAs, oncogenic or pathogenic gene loci in animals and human.

In some embodiments, the “gene-editing-sequence” comprises one or more sequences such as stop codons to deactivate target gene(s) and to eliminate undesirable effects of off-target edits.

In some embodiments, the “gene-editing-sequence” comprises one or more methylated nucleic acid bases.

In some embodiments, the “gene-editing-sequence” comprises one or more 5-methyl cytosines.

In some embodiments, the “gene-editing-sequence” comprises one or more 5-methyl cytosines, which belong to a CpG island.

In some embodiments, the “gene-editing-sequence” comprises DNA sequence(s) to correct oncogenic or pathogenic gene mutations in animals and human.

In other embodiments, the conjugated single strand HDR/SSTR donor template (ssDNA) forms a duplex with its complementary stand, i.e. HDR donor template is a double strand DNA (dsDNA) covalently linked to guide RNA(s) via a linker to either of its double strands.

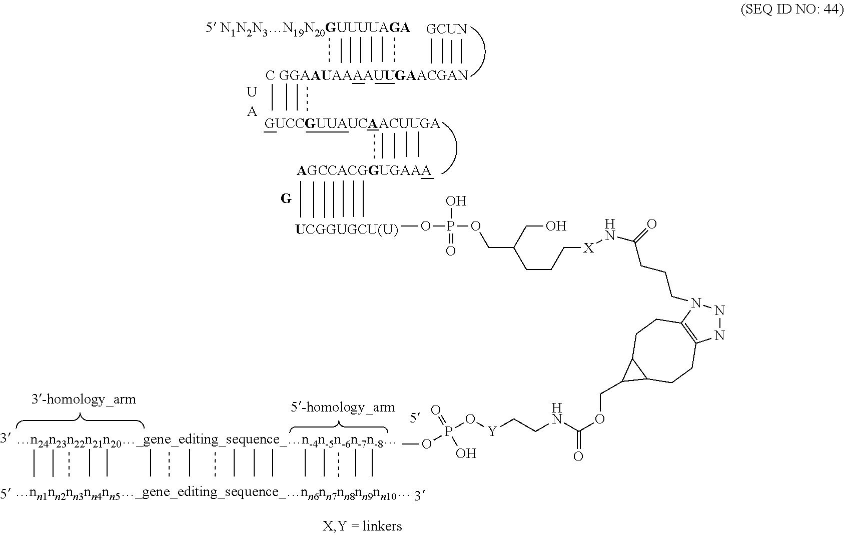

The said linker of X and Y is an L linker, a nucleotide linker or an nNt-linker and can be the same or different.

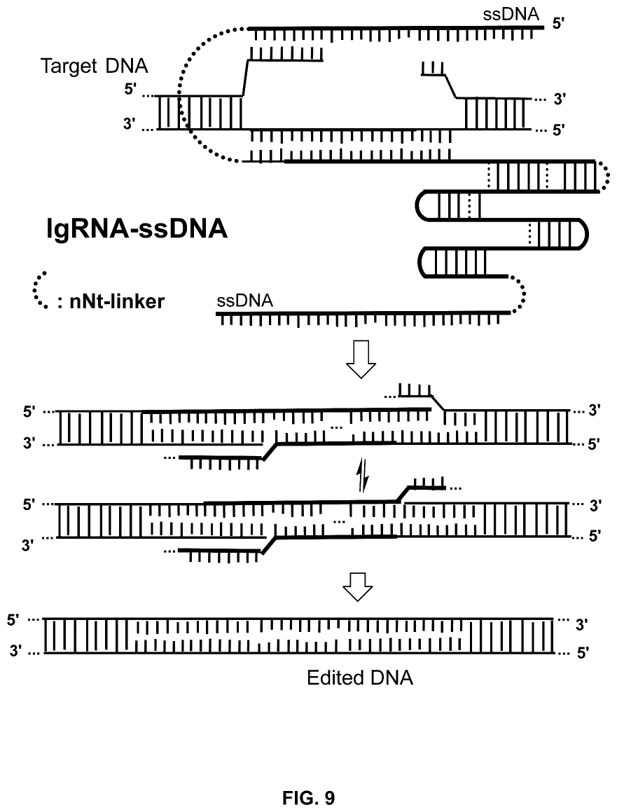

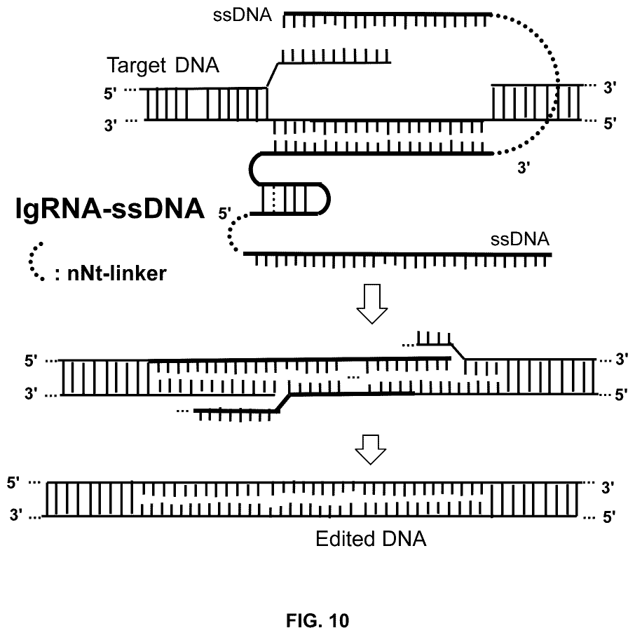

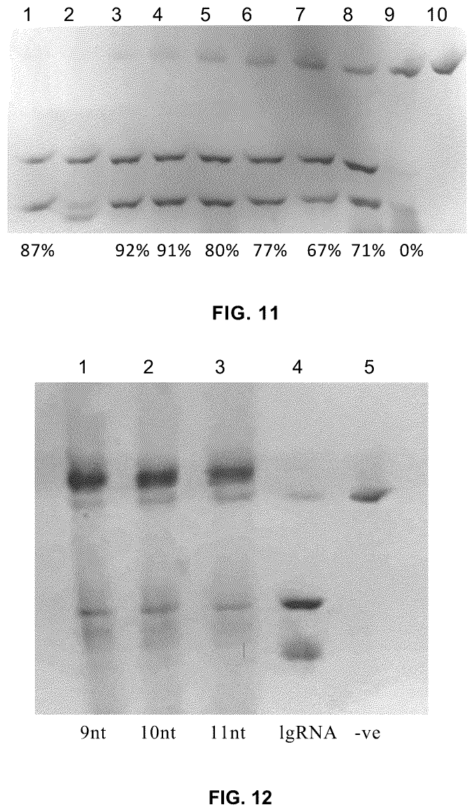

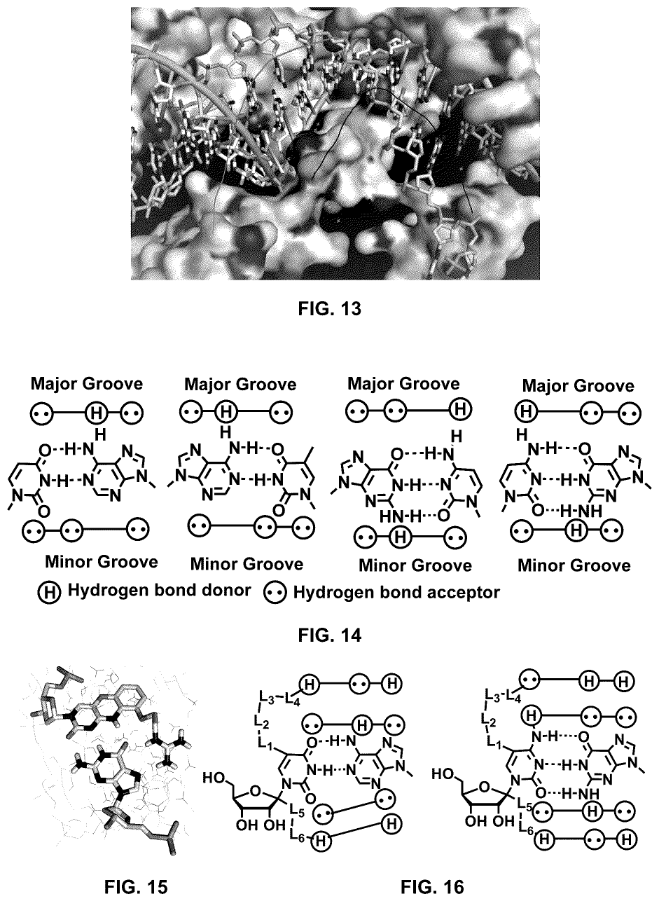

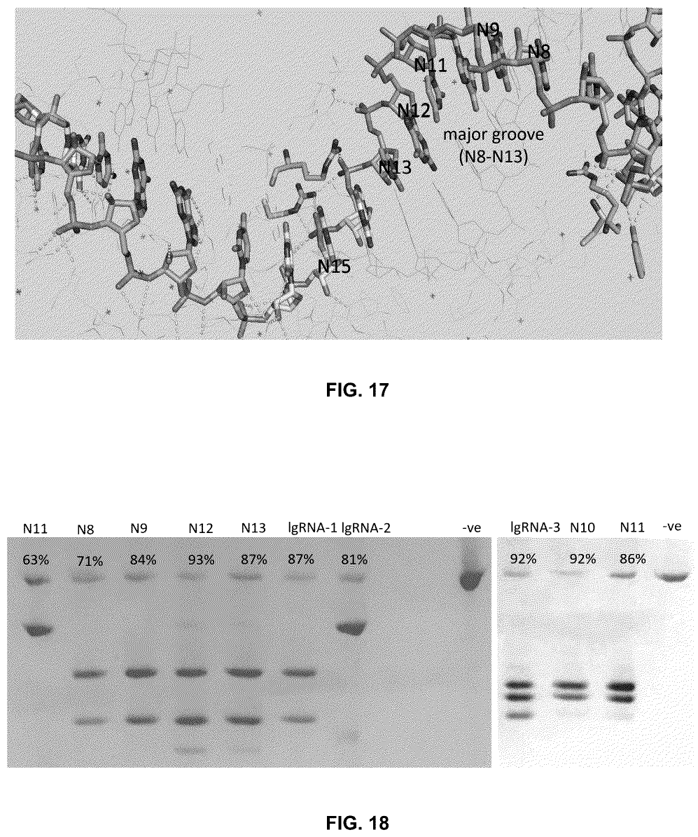

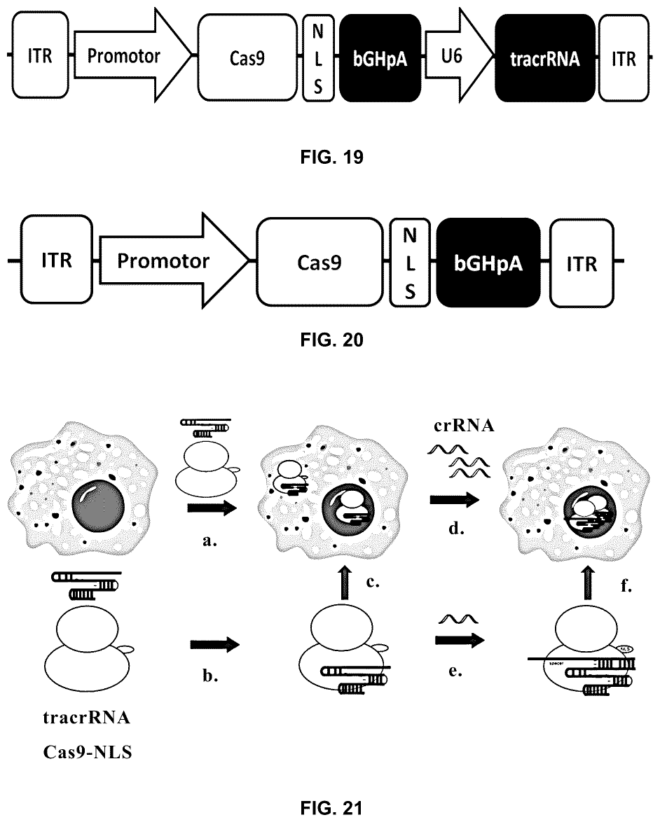

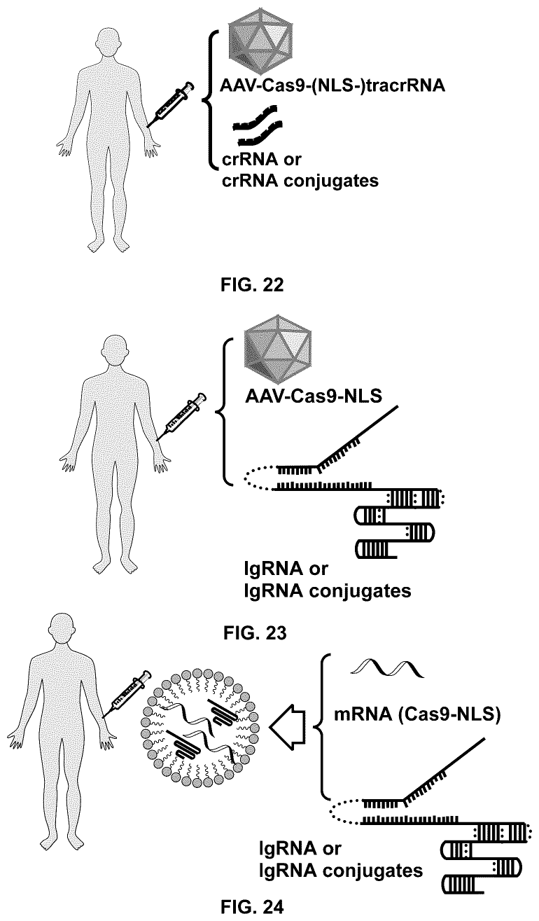

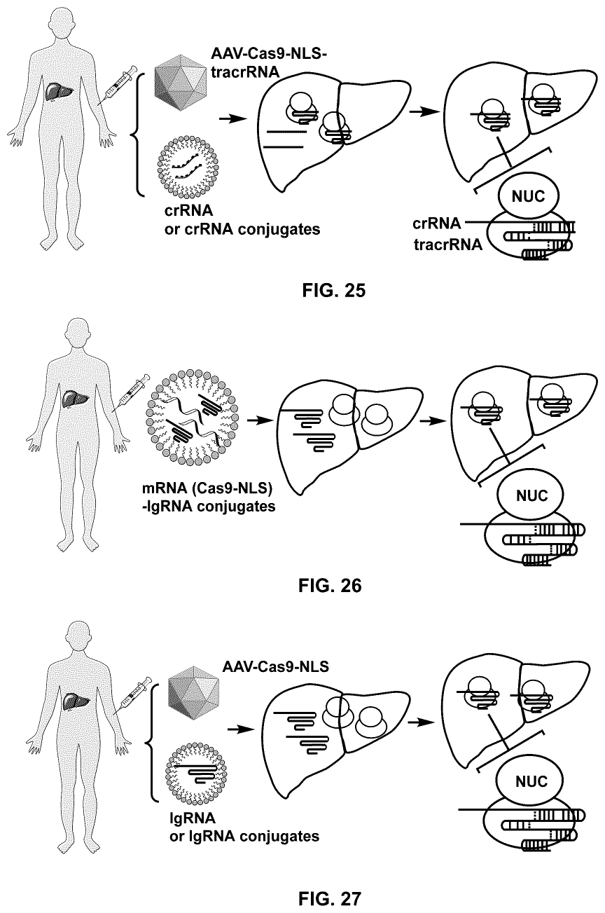

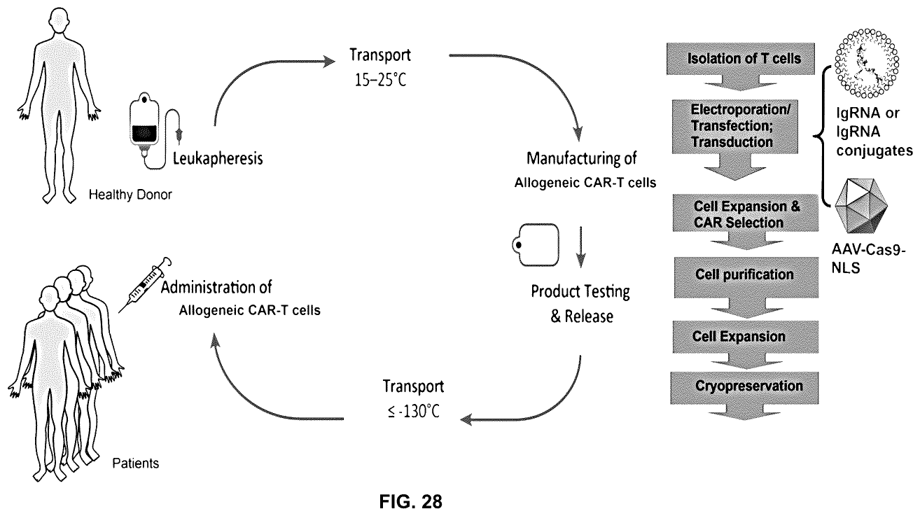

In some embodiments, the said conjugated oligonucleotide is a donor template which comprises two sequences overlapping with the target strand or with the non-target strand of the DNA duplex to be edited, e.g. 5′- and 3′-homology arms, and a gene editing sequence, and the said two sequences flanking the said gene editing sequence are optionally chemically modified, and the said 3′-homology arm comprises an optionally chemically modified DNA segment of 5 to 17 nucleotides complementary to 5′-end of the spacer of guide RNA, and is joined at its 5′-end by an oligonucleotide comprising said gene editing sequence and said 5′-homology arm as a template for DNA synthesis (i.e. FIGS. 9 and 10).

In some embodiments, the said conjugated oligonucleotide is a donor template which comprises two sequences overlapping with the target strand or with the non-target strand of the DNA duplex to be edited, e.g. 5′- and 3′-homology arms, and a gene editing sequence, and the said two sequences flanking the said gene editing sequence are optionally chemically modified, and the said 3′-homology arm comprises an optionally chemically modified chimeric DNA/RNA oligonucleotide segment of 5 to 17 nucleotides complementary to 5′-end segment of the cleaved DNA strand (containing 3′-OH) which is also the primer for chain extension, and is joined at its 5′-end by an oligonucleotide comprising said gene editing sequence and said 5′-homology arm as a template for DNA synthesis (i.e. FIGS. 9 and 10).

In some embodiments, the said conjugated oligonucleotide is a donor template which comprises two sequences overlapping with the target strand or with the non-target strand of the DNA duplex to be edited, e.g. 5′- and 3′-homology arms, and a gene editing sequence, and the said two sequences flanking the said gene editing sequence are optionally chemically modified, and the said 3′-homology arm comprises an optionally chemically modified DNA or chimeric DNA/RNA oligonucleotide segment of 7 to 14 nucleotides complementary to 5′-end segment of the cleaved DNA strand (containing 3′-OH) which is also the primer for chain extension, and is joined at its 5′-end by an oligonucleotide comprising said gene editing sequence and said 5′-homology arm as a template for DNA synthesis (i.e. FIGS. 9 and 10).

In some embodiments, the conjugated oligonucleotide is a donor template for DNA repair or serves as a bridging molecule to a second oligonucleotide via complementary hybridization.

In some embodiments, the lgRNA-ssDNA conjugate is further conjugated with one or more molecules such as PEG, non-PEG polymers, ligands for cellular receptors, lipids, oligonucleotides, antibodies, polysaccharides and peptides.

In some embodiments, the lgRNA is conjugated with an RNA segment comprising a template for a reverse transcriptase (RTT), a primer binding sequence (PBS) and an optional stabilization secondary structure (structure motif) in 5′→3′ direction (CONJ-6).

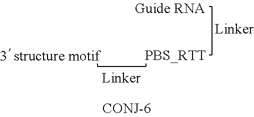

wherein the structure motif is evopreQ1, mpknot, a Zikavirus exoribonuclease-resistant RNA motif (xr-pegRNA), a G-quadruplex (G-PE), a stem-loop aptamer (sPE) or another structured nucleic acid (Chen, P. and Liu, D. Nat Rev Genet, 2023, 24, 161-177), and the conjugating linker “]” is a chemical nNt-linker or an oligonucleotide linker, and wherein “Guide RNA” is a chemically ligated guide RNA, an sgRNA or a crRNA, and PBS_RTT is a optionally modified single molecule RNA, and the Linker between “Guide RNA” and RTT is covalently joined at 3′-, 5′-terminal or a ligation site.

In some embodiments, at least one of the conjugating linkers “]” is a non-nucleotide linker.

In some embodiments, the structure motif is a chemically modified RNA for further stabilization (See examples: Glazier, D. A. and et al. Bioconjugate Chemistry, 2020, 31, 1213-1233), while the spacer sequence, sgRNA scaffold, PBS, and nucleotide linkers are each optionally chemically modified and RTT comprises nucleotide analogues compatible to DNA synthesis by reverse transcription.

In certain embodiments, PBS hybridizes with the primer which is the PAM distal fragment of the R-loop asymmetrically released from Cas9:lgRNA:DNA complexes after nicking to form a complementary duplex of 7-14 nt upstream and before a PAM proximal sequence (6-13 nt including cutting site, 5′→3′). The complementarity between RTT and the PAM proximal sequence (including PAM) is minimized by silent mutations (e.g., replacement with alternative codons).

In some embodiments, the structure motif is further conjugated at its 3′-end with one or more molecules such as PEG, non-PEG polymers, ligands for cellular receptors, lipids, oligonucleotides, antibodies, polysaccharides and peptides.

In some embodiments, the structure motif is further conjugated at its 3′-end with one or more peptides such as a cell penetrating peptide and a nuclear localization signal peptide.

### Non-Nucleotide Linkers (nNt-linker)

An nNt-Linker, formed by chemical ligation, comprises an M core structure of Formula M-1 to M-13 as non-limiting examples:

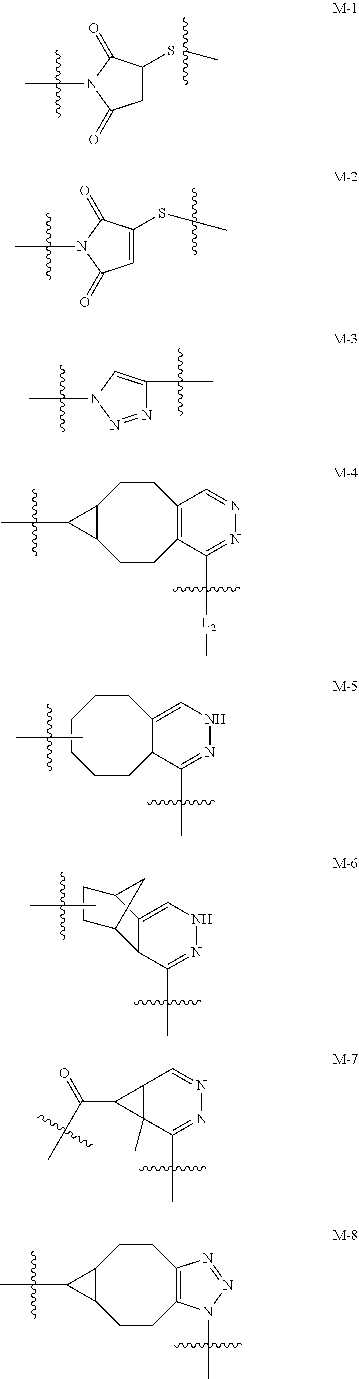

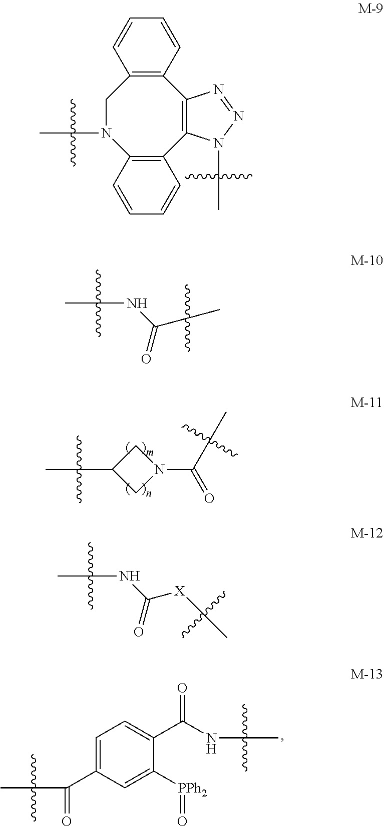

wherein X=O, S, NH, or CH₂, m=0 to 3 and n=0 to 3,
and two L linkers comprising Formula L-1 to L-23 as non-limiting examples:

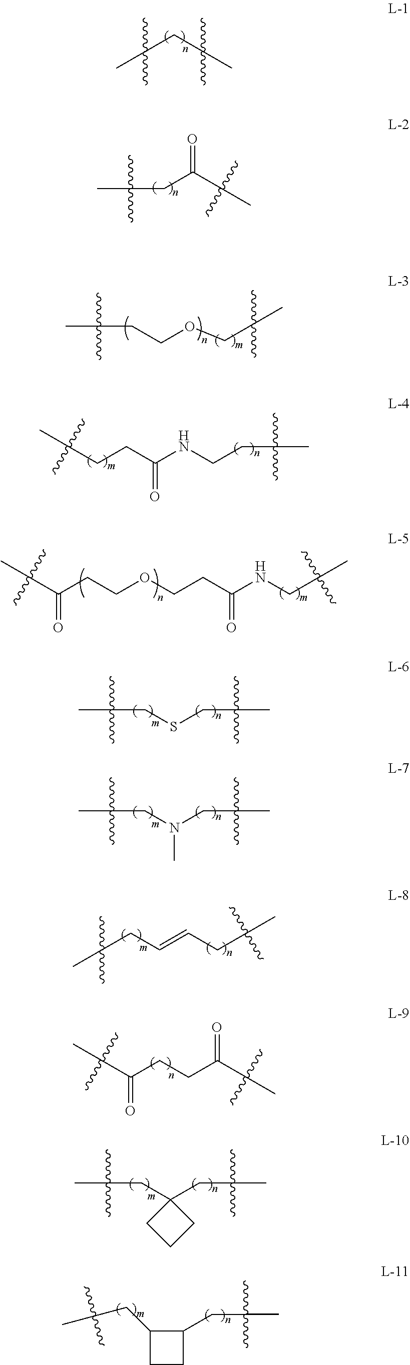

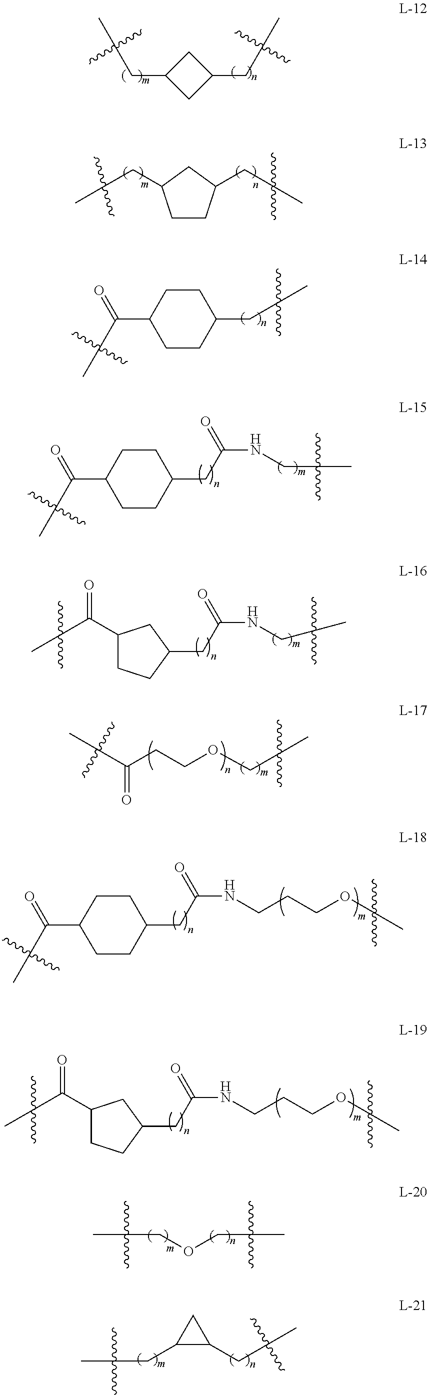

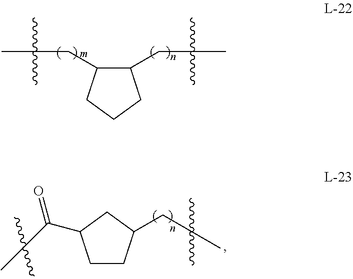

wherein m=0 to 16 and n=0 to 16,

said L linkers and said M core structure are joined as L-M-L, wherein the two L linkers are the same or different, and each L optionally comprises one or more structures of Formula L-1 to L-23 or partial structure(s), and attached to two terminal nucleotides of Formula Nuc-1 to Nuc-18 as non-limiting examples:

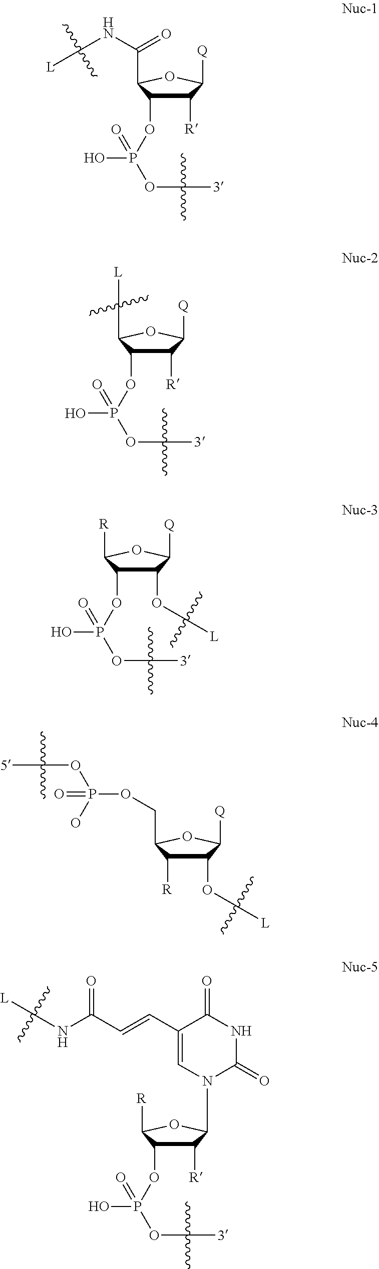

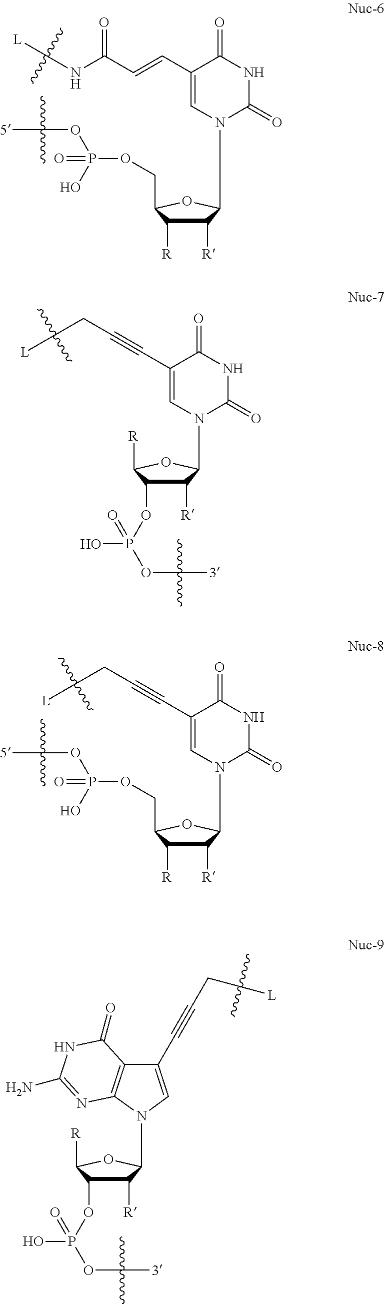

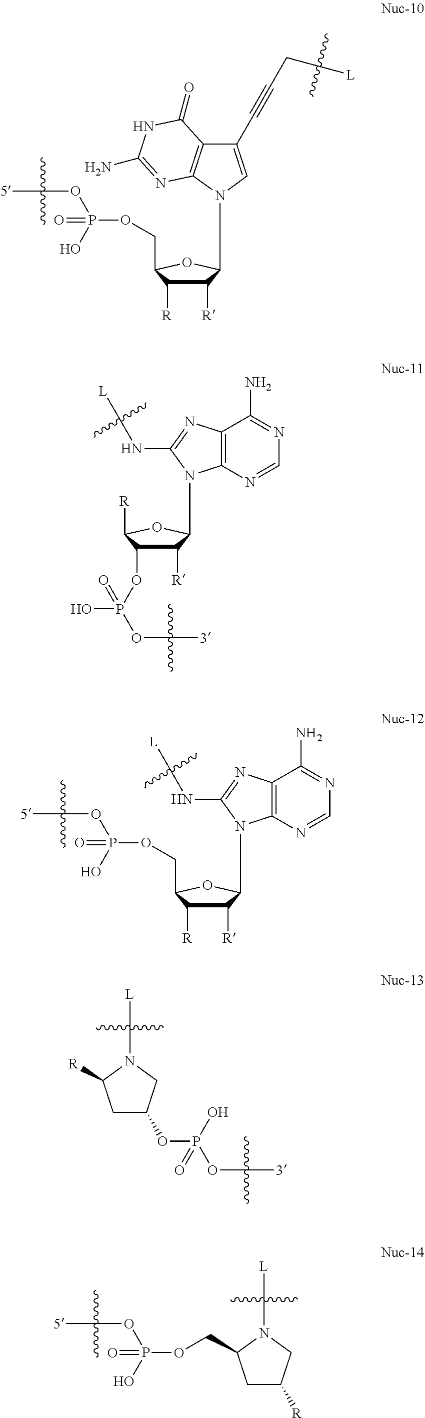

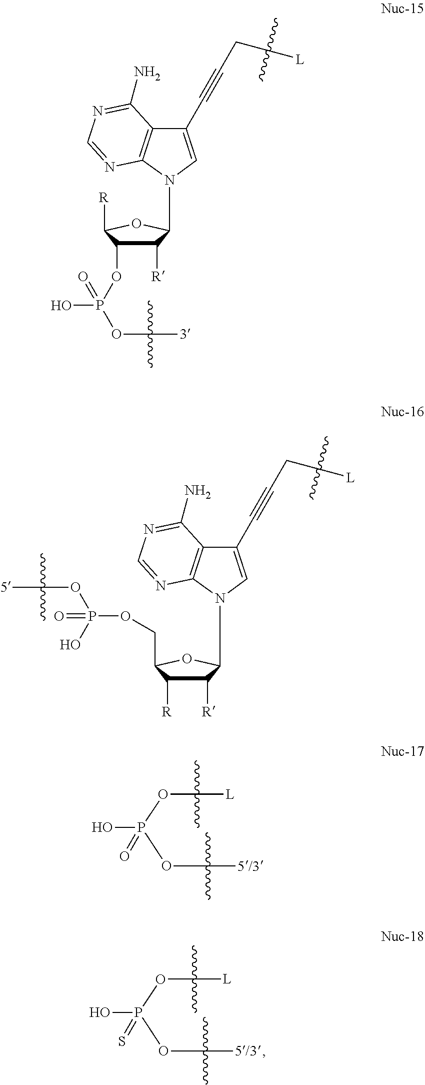

wherein the attached positions are

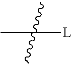

### to L-M-L and

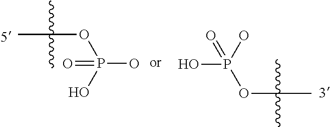 [SMILES: *OP(=O)(O)O ; *OP(=O)(O)O]

to upstream and downstream oligonucleotides, respectively, and wherein R is H, OH,

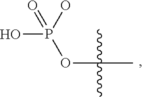 [SMILES: *OP(=O)(O)O]

### CH₂OH,

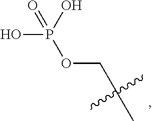 [SMILES: *COP(=O)(O)O]

F, NH₂, OMe, CH₂OMe, OCH₂CH₂OMe, an alkyl, a cycloalkyl, an aryl, or heteroaryl, R′ is H, OH,

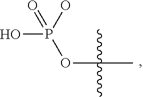 [SMILES: *OP(=O)(O)O]

### CH₂OH,

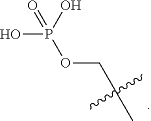 [SMILES: *COP(=O)(O)O]

F, NH₂, OMe, CH₂OMe, OCH₂CH₂OMe, an alkyl, a cycloalkyl, an aryl, or a heteroaryl, and Q is a natural or a non-natural nucleic acid base.

In some embodiments, the M core structure, L, and terminal nucleotides are optionally modified with substituents such as halogen (F, Cl, Br, I), lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, an optionally substituted aryl, an optionally substituted heteroaryl, an optionally substituted sulfonyl, or optionally substituted acyl, which includes but is not limited to C(═O) alkyl, NR′₂, CN, CO₂H, CO₂R′, CONH₂, CONHR′, CONR′₂, CH═CHCO₂H, or CH═CHCO₂R′, wherein R′is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, an nNt-Linker joins the 3′-terminal nucleotide of a crRNA and the 5′-terminal nucleotide of a tracrRNA. In some embodiments, an nNt-Linker joins the 5′-terminal nucleotide of a crRNA and the 3′-terminal nucleotide of a tracrRNA. In some embodiments, an nNt-Linker joins two oligonucleotide segments of tracrRNA.

In some embodiments, one of the two Ls in an nNt-linker (L-M-L) is covalently linked to guide RNA(s), and the other is covalently linked to a PEG polymer, a non-PEG polymer, a ligand for cellular receptors, a lipid, an oligonucleotide, an antibody, a polysaccharide or a peptide.

In some embodiments, one of the two Ls in nNt-linkers (L-M-L) is covalently linked to an exposed amino acid residue of Cas protein such as lysine, serine, and cysteine, and the other is covalently linked to a PEG polymer(s), a non-PEG polymer, a ligand for cellular receptors, a lipid, an oligonucleotide, an antibody, a polysaccharide(s) or a peptide.

In some embodiments, the nNt-linkers between the two nucleotides/nucleosides are represented by the following formulas:

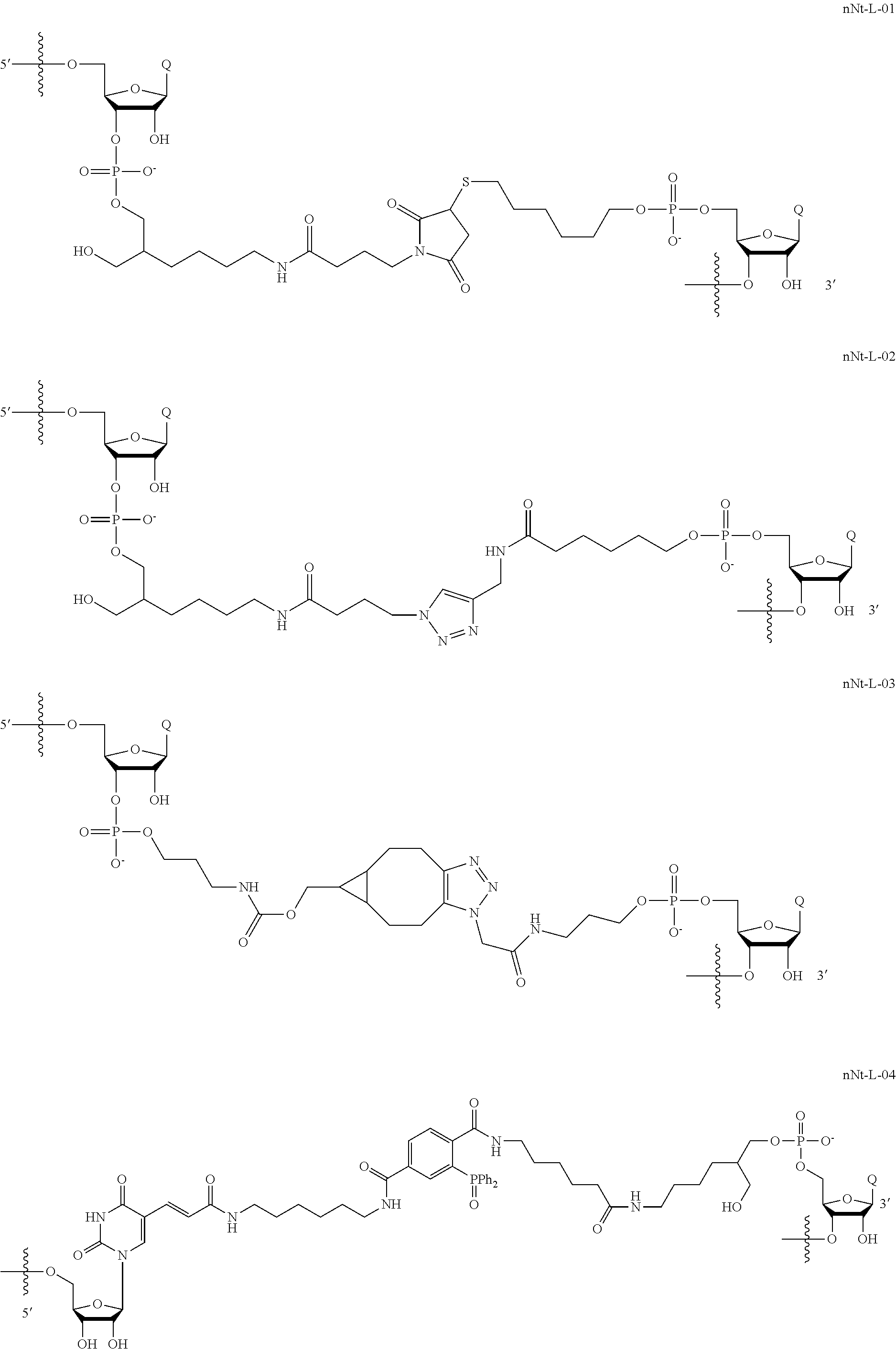

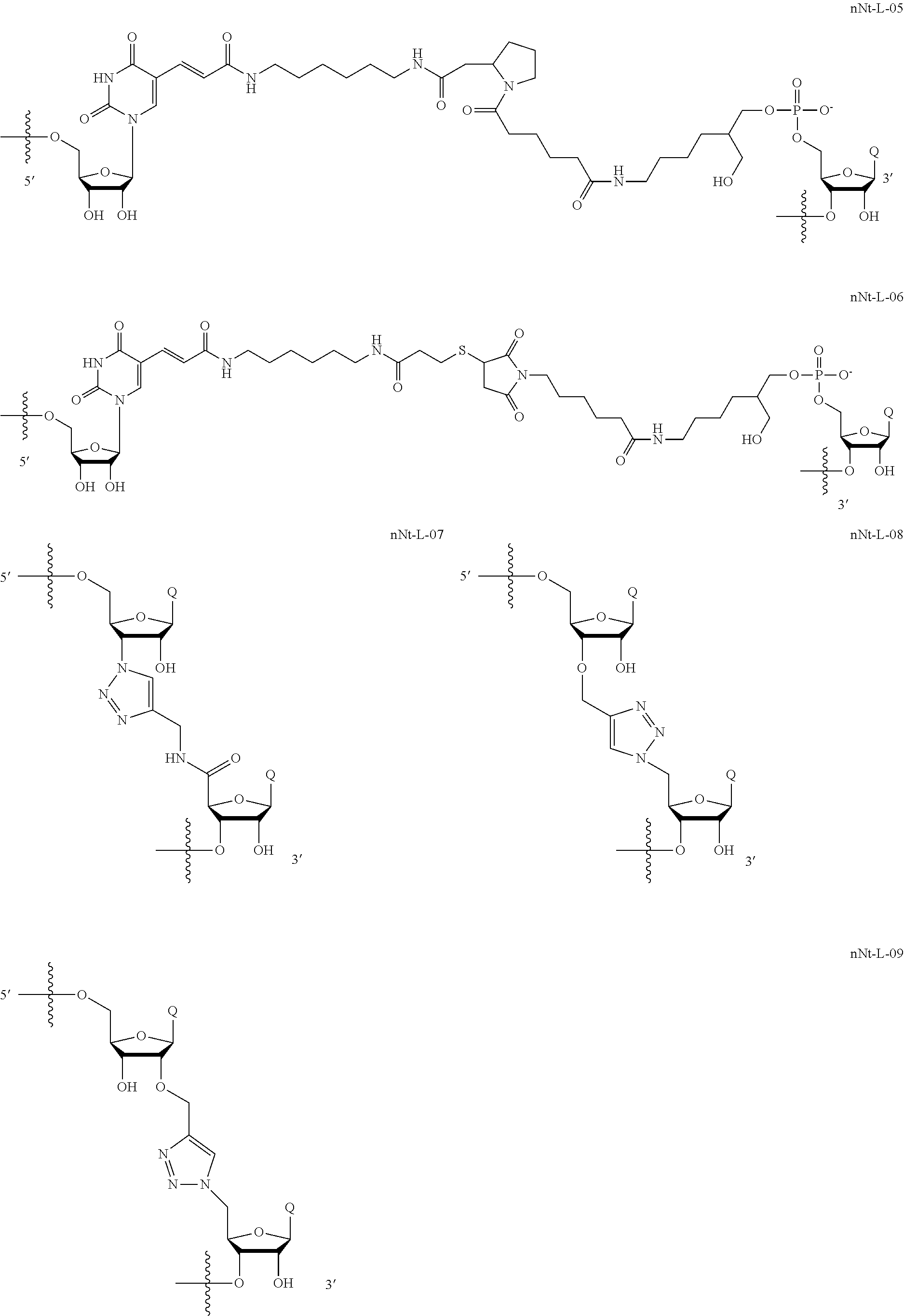

### CRISPR Effector Proteins

In some embodiments, CRISPR effector endonuclease is selected from Cas proteins of Type II, Class 2 including Streptococcus pyogenes-derived Cas9 (SpCas9, 4.1 kb), smaller Cas9 orthologues, including Staphylococcus aureus-derived Cas9 (SaCas9, 3.16 kb), Campylobacter jejuni-derived Cas9 (CjCas9, 2.95 kb), Streptococcus thermophilus Cas9 (St1Cas9, 3.3 kb), Neisseria meningitidis Cas9 (NmCas9, 3.2 kb), and many other variants of engineered Cas9 proteins such as SpCas9-HF1, eSpCas9, and HypaCas9, proteins of Type V, Class 2 including Cas12 (Cas12a (Cpf1), Cas12b (C2cl), Cas 12c, Cas12e, Cas12g, Cas12h, Cas12i, and etc.) and Cas 14, and proteins of Type VI, Class 2 such as Cas13a and Cas13b. The said CRISPR effector protein can be a nickase e.g. nCas9 such as a SpCas9-nickase (D10A or H840A), or a catalytically inactive protein e.g. dCas9 coupled/fused with a protein effector such as a DNA polymerase, FokI, transcription activator(s), transcription repressor(s), catalytic domains of DNA methyltransferase, histone acetyltransferase and deacetylase, reverse transcriptase (prime editor), and nucleic acid deaminases (base editor) at its either N- or C-terminal.

In another embodiment, the said CRISPR effector endonuclease is an artificial one comprising one or more functional domains derived from human.

In yet another embodiment, the said CRISPR effector endonuclease is a class 2 CRISPR Cas protein functionalized by site-directed mutagenesis to introduce orthogonal conjugating sites such as cysteines and remove deleterious conjugating sites (e.g. C80 in SpCas9), and corresponding RNP conjugates are prepared by selective conjugations such as PEGylation of cysteines by maleimide chemistry.

In yet another embodiment, the said CRISPR effector endonuclease is a class 2 CRISPR Cas protein fused with a human DNA or RNA polymerase via a peptide linker.

In yet another embodiment, the RNP complex conjugates are synthesized by reactions with crosslinking reagents, or by enzymes such as TGase which catalyzes amine transfer reaction between a primary amine at terminal side of PEG and the carboxamide of glutamine.

### Tissue Tropic Viral Vectors Encoding a Cas Protein or a Cas-TracrRNA Complex

In some embodiments, the said viral vector encoding a Cas effector endonuclease such as Cas9, or a Cas9-tracrRNA complex, is a retrovirus, lentivirus, adenovirus, AAV, or baculovirus.

In some embodiments, the said viral vector encoding a Cas effector endonuclease such as Cas9, or a Cas9-tracrRNA complex, is an engineered AAV or AAV chimera to enable high transduction efficiency at a targeted tissue by changing the tropism of AAV capsids and to have low immunogenicity by evading human preexisting anti-AAV capsid neutralizing antibodies.

In some embodiments, the said viral vector encoding a Cas effector endonuclease such as Cas9, or a Cas9-tracrRNA complex, is a native or an engineered AAV to enable brain tissue targeted delivery. Such AAV serotypes include as non-limiting examples AAV1, AAV2/DJ, AAV2/DJ8, AAV2g9, AAV2-retro and scAAV9.

In some embodiments, the said viral vector encoding a Cas effector endonuclease such as Cas9, or a Cas9-tracrRNA complex, is a native or an engineered AAV to enable liver tissue targeted delivery. Such AAV serotypes include as non-limiting examples AAV8 and AAV3.

In some embodiments, the said viral vector encoding a Cas effector endonuclease such as Cas9, or a Cas9-tracrRNA complex, is a native or an engineered AAV to enable muscle tissue targeted delivery. Such AAV serotypes include as non-limiting examples AAV6, AAV8 and AAV9.

In some embodiments, the said viral vector encoding a Cas effector endonuclease such as Cas9, or a Cas9-tracrRNA complex, is a native or an engineered AAV to enable heart tissue targeted delivery. Such AAV serotypes include as non-limiting examples AAV rh74 and AAV9.

In some embodiments, the said viral vector encoding a Cas effector endonuclease such as Cas9, or a Cas9-tracrRNA complex, is a native or an engineered AAV to enable retina tissue targeted delivery. Such AAV serotypes include as non-limiting examples AAV1, AAV2, AAV5, AAV8 and AAV9.

In some embodiments, the said viral vector encoding a Cas effector endonuclease such as Cas9, or a Cas9-tracrRNA complex, is a native or an engineered AAV to enable lung tissue targeted delivery. Such AAV serotypes include as non-limiting examples AAV9.

In some embodiments, the expression of a Cas effector endonuclease such as Cas9, or a Cas9-tracrRNA complex by the said viral vector, is driven by inducible tissue-specific promoters.

In some embodiments, the expression of a Cas effector endonuclease such as Cas9, or a Cas9-tracrRNA complex by the said viral vector, is driven by brain tissue-specific promoters such as pMecp2, hSyn1, TRE3G and EFS as non-limiting examples.

In some embodiments, the expression of a Cas effector endonuclease such as Cas9, or a Cas9-tracrRNA complex by the said viral vector, is driven by liver tissue-specific promoters such as TBG and HCRhAATp or by lung tissue-specific promoters such as EFS, as non-limiting examples.

In some embodiments, the expression of a Cas effector endonuclease such as Cas9, or a Cas9-tracrRNA complex by the said viral vector, is driven by heart tissue-specific promoters such as CMV, Myh6, CB and CK7-miniCMV as non-limiting examples.

In some embodiments, the expression of a Cas effector endonuclease such as Cas9, or a Cas9-tracrRNA complex by the said viral vector, is driven by retina tissue-specific promoters such as EFS, CMV, Spc512, pMecp2, and Picam2 as non-limiting examples.

In some embodiments, the expression of a Cas effector endonuclease such as Cas9, or a Cas9-tracrRNA complex by the said viral vector, is driven by muscle tissue-specific promoters such as CMV, EFS and CK8 as non-limiting examples.

In some embodiments, the said viral vector encoding a Cas effector endonuclease such as Cas9, or a Cas9-tracrRNA complex, is administrated locally or systematically to optimize tissue targeted deliveries in animals and human. The injection site is intravenous, subcutaneous, intramuscular, intradermal, intraperitoneal, intravitreal, intranasal, intratracheal, subretinal, intraocular, or intracardiac, or at other sites. Chemically modified lgRNA(s), sgRNA(s), crRNA(s), or their conjugates are administrated either in an aqueous solution gymnotically or with transfection reagents or in a non-viral carrier by co-injections or separate injections.

In some embodiments, the said viral vector encoding a Cas effector endonuclease such as Cas9, or a Cas9-tracrRNA complex, is administrated to isolated cells including T cells, and chemically modified lgRNA(s), crRNA(s) or their conjugates are administrated either in an aqueous solution gymnotically by electroporation or microinjection, or with transfection reagents or in a non-viral carrier.

In some embodiments, to reduce potential off-targets due to stable expression of Cas or Cas fusion proteins when the proteins are delivered in viral vectors such as AAV, one or more NLS sequences are added to guide RNA conjugates only. Reposition of NLS sequence(s) from proteins to guide RNA conjugates prevents the guide-independent off-target activities of Cas and Cas-effector fusion proteins (unbound by guide RNA conjugates) in cell nuclei and enables controlled gene manipulations by controlled dosing of guide RNA-peptide conjugates.

### Non-Viral Delivery of RNP Complexes

In some embodiments, guide RNA conjugates, conjugates of guide RNA(s)-Cas protein (RNP) complexes such as PEGylated RNP complexes, or mixed lgRNA conjugate(s) and an mRNA encoding a Cas-NLS or Cas protein are delivered with lipids well used in delivery of therapeutic nucleic acids such as DOPE, DOTAP, DOPS, DLin-MC3-DMA, cKK-E12, DSPC, POPE, 503013, PEG-DMG, bio-reducible lipid 8-014B, cholesterol, and etc.

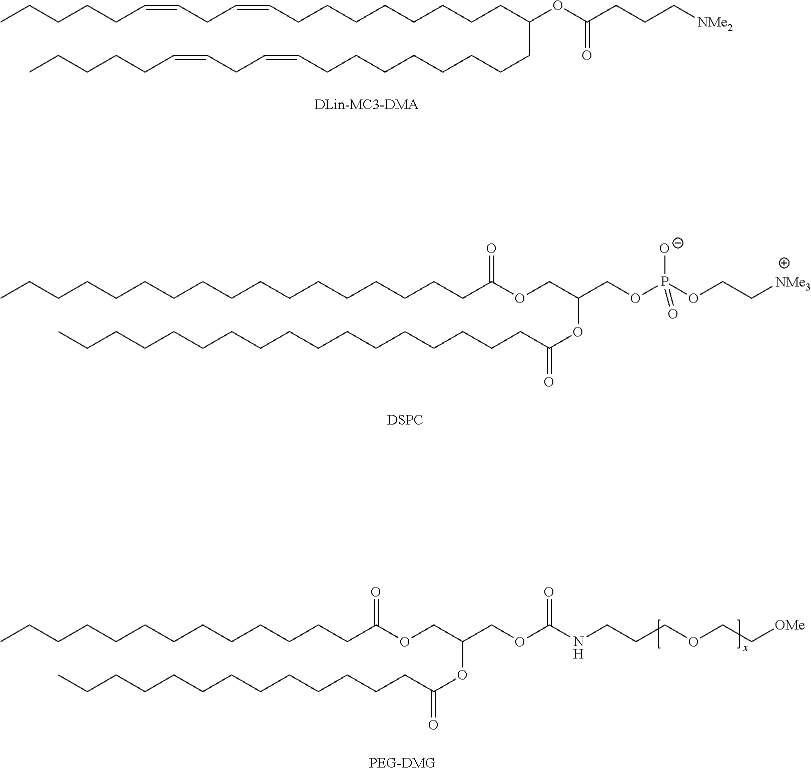

In other embodiments, conjugates of guide RNA(s)-Cas protein (RNP) complexes such as PEGylated RNP complexes are delivered within lipid nanoparticles, liposomes, or exosomes.

In yet another embodiment, conjugates of guide RNA(s) and an mRNA encoding a Cas protein are delivered within lipid nanoparticles, liposomes, or exosomes.

In yet another embodiment, conjugates of guide RNA(s) are delivered with lipids or peptides, or within lipid nanoparticles, liposomes, or exosomes to cells stably or inducibly expressing a Cas protein, or to cells with a viral vector encoding a Cas protein co-delivered.

In yet another embodiment, conjugates of crRNA(s) are delivered with lipids or peptides, or within lipid nanoparticles, liposomes, or exosomes to cells stably or inducibly expressing both a Cas9 protein and a tracrRNA, or to cells with a viral vector encoding both a Cas9 protein and a tracrRNA co-delivered.

In some embodiments, the said crRNA(s), or lgRNAs, or sgRNAs, or their conjugates or conjugates of guide RNA(s)-Cas protein (RNP) complexes are administrated by electroporation or microinjection.

### Conjugates of RNP Complexes

An aspect of the invention is directed to conjugates of guide RNA(s)-Cas protein (RNP) complexes such as a PEGylated CRISPR Cas protein-guide RNA complex(es), which are prepared by conjugation of a preformed guide RNA-CRISPR-Cas RNP(s) (RNP-I as an example),

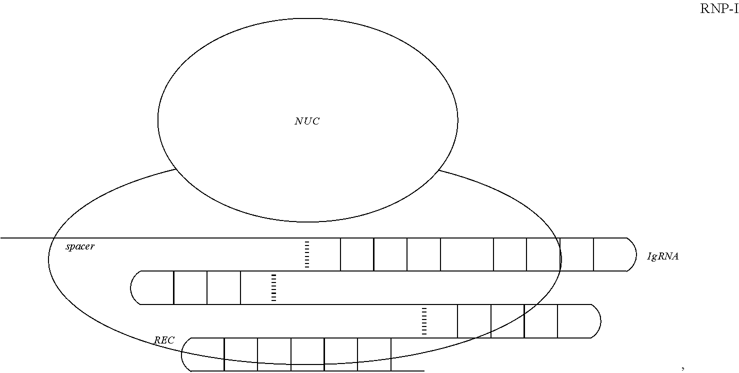

or prepared by conjugation of a Cas protein engineered for site-selective conjugations followed by complexing with a guide RNA(s) to form an RNP complex, wherein guide RNA(s) is an lgRNA, a crRNA, an sgRNA, or dual guide RNAs, and Cas protein is Cas9 comprising a nuclease lobe (NUC) and a recognition lobe (REC), an engineered Cas9, or a Cas protein other than the example of S. pyogenes Cas9 represented here; wherein lgRNA is composed of dual-modules (crRNA and tracrRNA) and a single nNt-linker, dual or multiple nNt-linkers (lgRNA) formed by chemical ligation(s) and the ligation sites are preferably located at tetraloop (A32-U37) and/or stem 2 (C70-G79) (of reported sgRNA engineered for S. pyogenes Cas9), while any 3′, 5′-phosphodiester of sgRNA can be replaced by an nNt-Linker as a ligation site; and wherein guide RNA(s) is chemically modified and optionally conjugated with one or more molecules selected from a group comprising PEG, non-PEG polymers, ligands for cellular receptors, lipids, oligonucleotides, antibodies, polysaccharides and peptides.

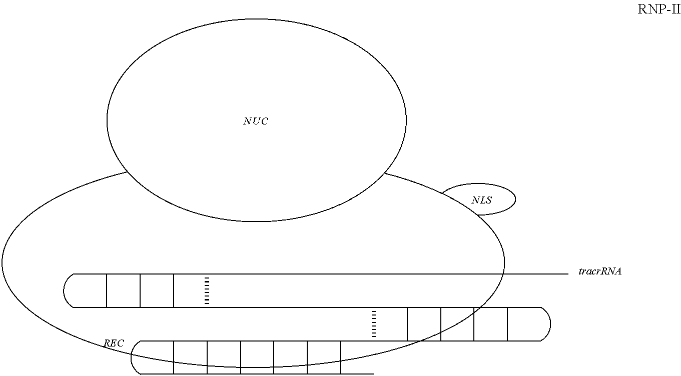

Another aspect of the invention is directed to viral vectors or plasmids, encoding binary CRISPR-Cas9-tracrRNA RNP complexes (RNP-II as an example) which further complex with crRNAs or crRNA conjugates in targeted cells, wherein Cas9 comprising a nuclease lobe (NUC) and a recognition lobe (REC) can be a Cas protein other than the example of S. pyogenes Cas9 represented here, and can be an engineered Cas protein.

Another aspect of the invention is directed to a PEGylated CRISPR Cas protein-guide RNA complex(es), wherein the guide RNA(s) is a crRNA, dual guide RNAs, an sgRNA or an lgRNA.

Another aspect of the invention is directed to a CRISPR Cas protein conjugated with one or more molecules selected from a group comprising PEG, non-PEG polymers, ligands for cellular receptors, lipids, oligonucleotides, antibodies, polysaccharides and peptides.

Another aspect of the invention is directed to a conjugate of CRISPR Cas protein-guide RNA complex(es), wherein the guide RNA(s) is a conjugate of a crRNA, dual guide RNAs, an sgRNA or an lgRNA with one or more single strand DNAs (ssDNA) as a donor template for gene editing.

Another aspect of the invention is directed to a conjugate of CRISPR Cas protein-guide RNA complex(es), wherein the said guide RNA(s) is a conjugate of a crRNA, dual guide RNAs, an sgRNA or an lgRNA with one or more single strand DNAs as a donor template for gene editing, and the CRISPR Cas protein is conjugated with one or more molecules selected from a group comprising PEG, non-PEG polymers, ligands for cellular receptors, lipids, oligonucleotides, antibodies, polysaccharides and peptides.

Some non-limiting examples of PEGylating reagents are given below.

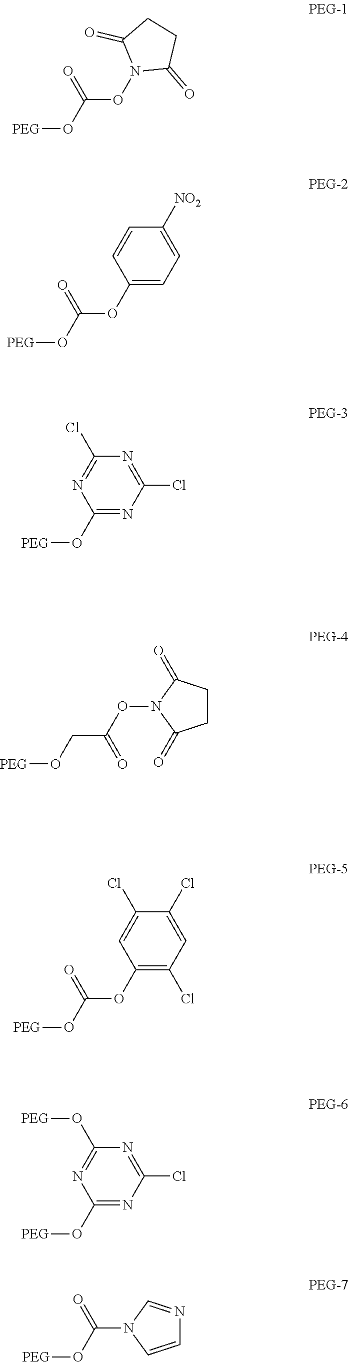

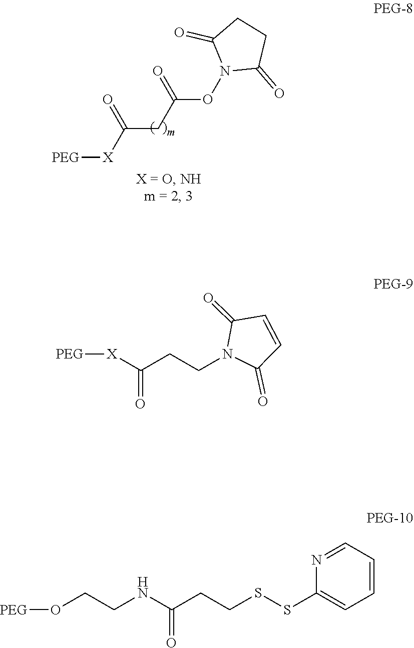

The PEG can be either linear or branched and conjugated at a lysine or cysteine residue:

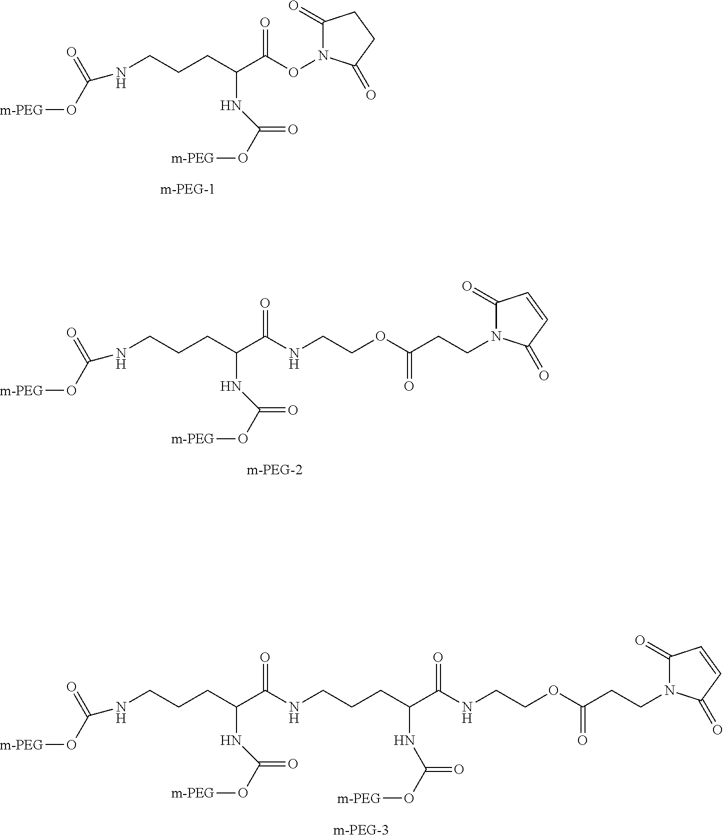

Another aspect of the invention is directed to a lipid conjugated Cas protein-guide RNA complex, which is prepared by lipid conjugation of a preformed CRISPR-Cas RNP complex or by lipid conjugation of a Cas protein engineered for site-selective conjugations followed by complexing with a guide RNA to form an RNP complex.

Some non-limiting examples of lipid conjugating reagents are given below:

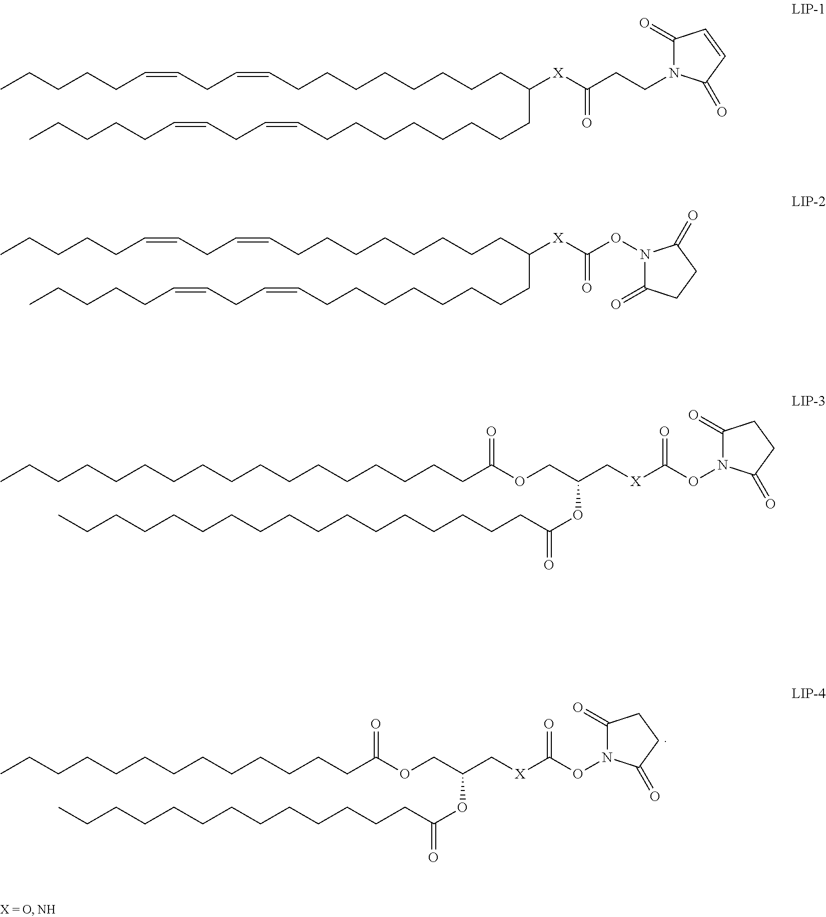

Another aspect of the invention is directed to a peptide conjugated Cas protein-guide RNA complex, which is prepared by peptide conjugation of a preformed CRISPR-Cas RNP complex or by peptide conjugation of a Cas protein engineered for site-selective conjugations followed by complexing with a guide RNA to form an RNP complex. Non-limiting examples include conjugates of short peptides of nuclear localization signal (NLS).

Another aspect of the invention is directed to a Cas protein-guide RNA complex conjugated with small molecule ligands of cellular receptors, which are prepared by ligand conjugation of a preformed CRISPR-Cas RNP complex or by ligand conjugation of a Cas protein engineered for site-selective conjugations followed by complexing with a guide RNA to form a RNP complex. Non-limiting examples include asialoglycoprotein receptor ligands (ASGPrL) such as N-acetylgalactosamine (GalNAc) and lactobionic acid.

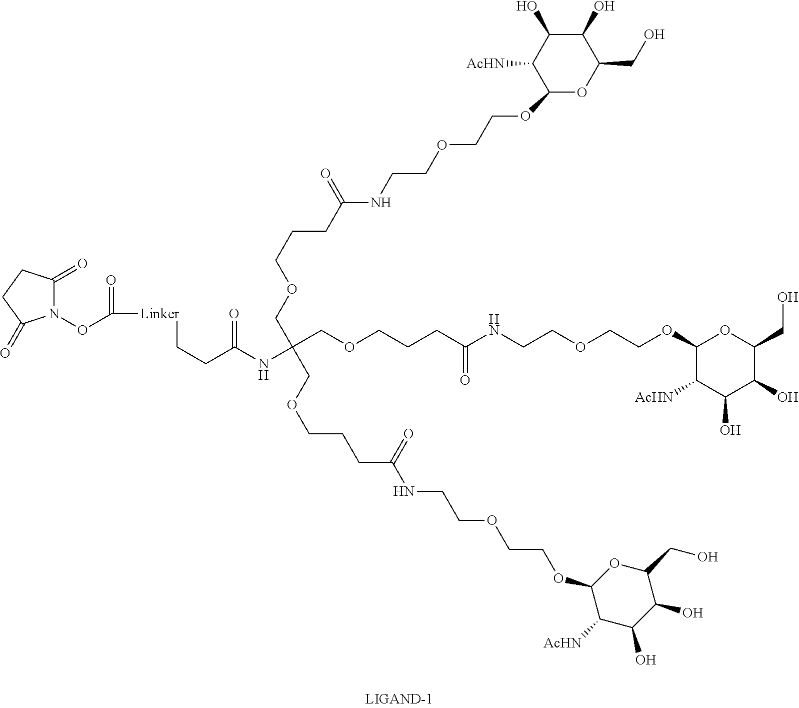

Yet another aspect of the invention is directed to a Cas protein-guide RNA complex conjugated with carbohydrates, which is prepared by carbohydrate conjugation of a preformed CRISPR-Cas RNP complex or by carbohydrate conjugation of a Cas protein engineered for site-selective conjugations followed by complexing with a guide RNA to form an RNP complex. Non-limiting examples include oligosaccharides and polysaccharides.

Yet another aspect of the invention is directed to conjugates of an engineered Cas9 protein mutated at C80 and incorporating one or more cysteines for site-selective conjugations by site-directed mutagenesis.

The present invention relates to compositions of conjugates of a guide RNA(s)-Cas protein (RNP) complex and their uses as medicinal agents in treatment of viral infectious diseases and as gene regulation, disruption and/or correction-based therapeutics. The conjugate(s) comprises a guide RNA-Cas protein (RNP) complex and one or more molecules selected from a group comprising PEG, non-PEG polymers, ligands for cellular receptors, lipids, oligonucleotides, antibodies, polysaccharides, glycans and peptides and chemically linked to a Cas protein and/or guide RNA(s).

The said uses may optionally include delivering an HDR template, wherein the template provides expression of a normal or less aberrant form of the protein to alleviate or eliminate a condition or disease state. The said HDR template may optionally be conjugated to guide RNA(s) and/or Cas protein.

Some aspects of the invention are directed to a donor template containing a “gene-editing-sequence” comprising one or more gene regulatory sequences or stop codons, and in some embodiments, the donor template(s) is conjugated to guide RNA(s) as part(s) of an lgRNA.

Some aspects of the invention are directed to a donor template containing a “gene-editing-sequence” comprising absent, one or more 5-methylcytosines (thus either hypermethylated or hypomethylated), and in some embodiments, the 5-methylcytosine donor template(s) is conjugated to guide RNA(s) as part(s) of an lgRNA.

Some aspects of the invention are directed to PEGylated CRISPR-Cas9-lgRNA-conjugate and transfection reagent(s) systems.

Some aspects of the invention are directed to the use of conjugates of a guide RNA(s)-Cas protein (RNP) complex such as a PEGylated CRISPR-Cas9-lgRNA-conjugate system for antiviral therapy targeting against proviral DNAs or integrated viral DNA and episomal circular DNAs.

In some embodiments, non-limiting examples of targeted viral genomic sequences of HBV include:

(SEQ ID NO: 45)
ctctgctagatcccagagtg [aGG],
(SEQ ID NO: 46)
gctatcgctggatgtgtctg [cGG],
(SEQ ID NO: 47)
tggacttctctcaattttct [a[G[G]G]G]],
(SEQ ID NO: 48)
gggggatcacccgtgtgtct [tGG],
(SEQ ID NO: 49)
tatgtggatgatgtggtactgg [gGG],
(SEQ ID NO: 50)
cctcaccatacagcactc [gGG],
(SEQ ID NO: 51)
gtgttggggtgagttgatgaatc [tGG],

wherein nucleotides in [ ] are PAMs, or any sequence of 17-20 nt immediately next to a PAM sequence.

In some embodiments, the therapeutic Cas9-lgRNA RNP conjugates comprise multiple lgRNAs targeting at different sites in viral genome (multiplex editing).

In some embodiments, multiple crgRNAs, including but not limited to sequences of YMDD and its mutations at catalytic domain of HBV polymerase, corresponding to

(SEQ ID NO: 52)
tatgtggatgat gtggtactgg [gGG],
(SEQ ID NO: 53)
tatatggatgat gtggtattgg [gGG],
(SEQ ID NO: 54)
tatgtggatgat gtggtattgg [gGG],
(SEQ ID NO: 55)
tatatagatgat gtggtactgg [gGG],

are ligated to tracrgRNA to result in a mixture of lgRNAs or lgRNA conjugates, and thus a mixture of Cas9-lgRNA RNP conjugates to target drug resistance in therapies based on direct-acting antiviral agents (DAA).

In some embodiments, non-limiting examples of targeted viral genomic sequences of HIV include:

(SEQ ID NO: 56)
gattggcaga actacacacc [aGG],
(SEQ ID NO: 57)
atcagatatc cactgacctt [tGG],
(SEQ ID NO: 58)
gcgtggcctg ggcgggactg [gGG],
(SEQ ID NO: 59)
cagcagttct tgaagtactc [cGG],

wherein nucleotides in [ ] are PAMs, or any sequence of 17-20 nt immediately next to a PAM sequence.

In some embodiments, non-limiting examples of targeted viral genomic sequences of herpesviridae virus such as HSV and EBV include:

(SEQ ID NO: 60)
gccctggaccaacccggccc [gGG],
(SEQ ID NO: 61)
ggccgctgccccgctccggg [tGG],
(SEQ ID NO: 62)
ggaagacaatgtgccgcca [tGG],
(SEQ ID NO: 63)
tctggaccagaaggctccgg [cGG],
(SEQ ID NO: 64)
gctgccgcggagggtgatga [cGG],
(SEQ ID NO: 65)
ggtggcccaccgggtccgct [gGG],
(SEQ ID NO: 66)
gtcctcgagggggccgtcgc [gGG],

wherein nucleotides in [ ] are PAMs, or any sequence of 17-20 nt immediately next to a PAM sequence.

In some embodiments, non-limiting examples of targeted genomic sequences include 17-20 nt of genes encoding endogenous T-cell receptors (TCR), HLA class I (HLA-I) or T-cell inhibitory receptors or signal molecules such as programmed cell death protein 1 (PD1) and cytotoxic T lymphocyte-associate protein 4 (CTLA4), immediately next to a PAM sequence.

Other aspects of the invention are directed to the use of said CRISPR-Cas-guide RNA conjugate systems for gene therapy and treatments of human infectious diseases.

In some embodiments, targeted sequences are human genomic sequences incorporating disease causing mutations, e.g.

(SEQ ID NO: 67)
5′-aaa gaa aat atm mmt ggt gtt-3′,

wherein m is a mutation, and the “gene-editing-sequence” comprises

(SEQ ID NO: 68)
5′-aaa gaa aat ate ttt ggt gtt-3′.

Non-limiting examples of such human single-gene disorders are given below (Table 1). Other examples include human polygenic disorders such as heart disease and diabetes.

TABLE 1
Examples single-gene disorders and their prevalence

| | |
|---|---|
| | Prevalence |
| Disorder | (approximate) |

Autosomal dominant

| | |
|---|---|
| Familial hypercholesterolemia | 1 in 500 |
| Polycystic kidney disease | 1 in 1250 |
| Neurofibromatosis type I | 1 in 2,500 |
| Hereditary spherocytosis | 1 in 5,000 |
| Marfan syndrome | 1 in 4,000 |
| Huntington's disease | 1 in 15,000 |

Autosomal recessive

| | |
|---|---|
| Sickle cell anaemia | 1 in 625 |
| Cystic fibrosis | 1 in 2,000 |
| Tay-Sachs disease | 1 in 3,000 |
| Phenylketonuria | 1 in 12,000 |
| Mucopolysaccharidoses | 1 in 25,000 |
| Lysosomal acid lipase deficiency | 1 in 40,000 |
| Glycogen storage diseases | 1 in 50,000 |

X-linked

| | |
|---|---|
| Duchenne muscular dystrophy | 1 in 7,000 |
| Hemophilia | 1 in 10,000 |

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates that may need to be independently confirmed.

This disclosure is not limited to particular embodiments described, as such may vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### EXAMPLES

The following examples further illustrate embodiments of the disclosed invention, which are not limited by these examples.

In addition, alternative structures of lgRNAs and their conjugates joined by two or more triazole nNt-linkers instead of orthogonal thioether/triazole linkers (EXAMPLES 11, 12 and 13) can be better prepared by a next-generation synthesis using a combination of nucleic acid chain extension on an azido substituted solid support (to avoid challenging post-synthesis NHS chemistry) and sequential triazole-formation-mediated ligations enabled by an efficient regio-specific diazo transfer reaction (WO2023/101993 and US2023/167441 are cited herein and incorporated by reference in their entirety).

### Example 1: Synthesis of SaCas9 crRNA-GalNAc Conjugate

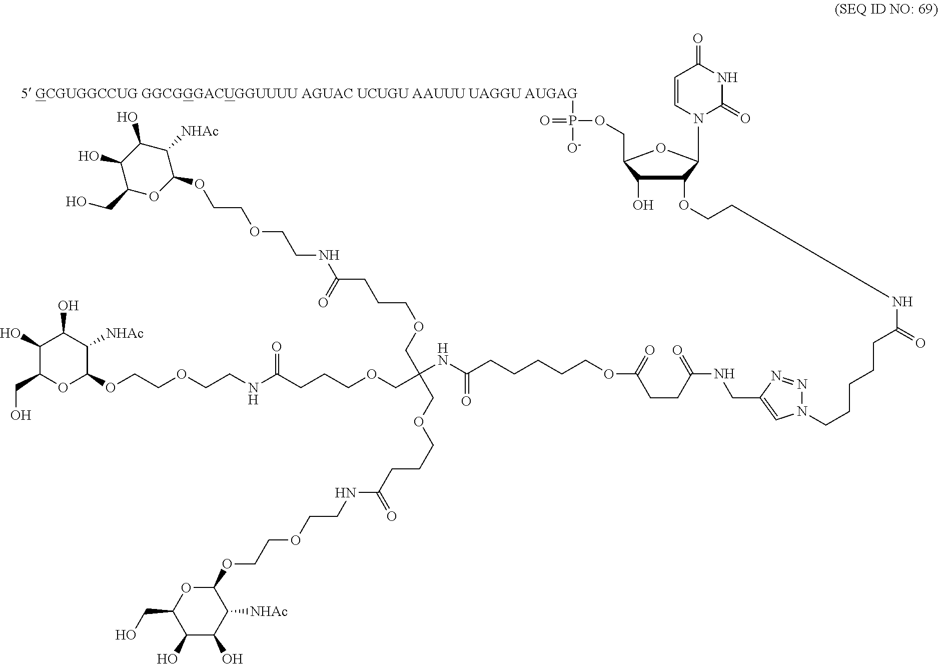

STEP 1. The 3′-amino-crRNA is prepared using 2′-TBS protected RNA phosphoramidite monomers with t-butylphenoxyacetyl protection of the A, G and C nucleobases and unprotected uracil. 0.3 M Benzylthiotetrazole in acetonitrile (Link Technologies) is used as the coupling agent, t-butylphenoxyacetic anhydride as the capping agent and 0.1 M iodine as the oxidizing agent. Oligonucleotide synthesis is carried out on an Applied Biosystems 394 automated DNA/RNA synthesizer using the standard 1.0 μmole RNA phosphoramidite cycle. Uridine on CPG is prepared as before (US20160215275A1 is cited herein and incorporated by reference in their entirety) and packed into a twist column. All β-cyanoethyl phosphoramidite monomers are dissolved in anhydrous acetonitrile to a concentration of 0.1 M immediately prior to use. Stepwise coupling efficiencies are determined by automated trityl cation conductivity monitoring and in all cases are >97.5%.

Fmoc is then cleaved by treatment with 20% piperidine in DMF. The resulting 3′-end aminoethyl oligonucleotide is then treated with NHS ester of 6-azido caproic acid in DMF.

Cleavage of oligonucleotides from the solid support and deprotection are achieved by exposure to concentrated aqueous ammonia/ethanol (3/1 v/v) for 2 h at room temperature followed by heating in a sealed tube for 45 min at 55° C. and desilylation in 1.0 M TBAF in THF for 24 h to give 3′-azide-crRNA.

Alternatively, the step of reaction with NHS ester is skipped, and the above resulting fully deprotected oligonucleotide is dissolved in 0.5 M Na₂CO₃/NaHCO₃ buffer (pH 8.75) and incubated with succinimidyl-6-azidohexanate (20 eq.) in DMSO to give 3′-azide-crRNA.

STEP 2. To a solution of tri-GalNAc-alkyne and 3′-azide-crRNA (0.2 nmol of each) in 0.2 M NaCl (50 μL) at room temperature are added tris-hydroxypropyl triazole ligand (28 nmol in 42 μL 0.2 M NaCl), sodium ascorbate (40 nmol in 4 μL 0.2 M NaCl) and CuSO₄·5H₂O (4 nmol in 4.0 μL 0.2 M NaCl), under argon. The reaction mixture is kept under argon at room temperature for the desired time, and formamide (50 μL) is added. The reaction is analyzed by loading directly onto a 20% polyacrylamide electrophoresis gel, and purified by reversed-phase HPLC to give the crRNA-tri-GalNAc conjugate.

### Example 2: Cas9::crRNA-GalNAc::tracrRNA Complex

Cas9 protein:

Recombinant Cas9 protein is available from New England BioLabs, Inc. and other providers or is expressed and purified from E. coli by a routinely used protocol (Anders, C. and Jinek, M. Methods Enzymol. 2014, 546, 1-20). The purity and concentration of Cas9 protein are analyzed by SDS-PAGE.

Cas9::crRNA-GalNAc::tracrRNA complex:

tracrRNA is synthesized on an Applied Biosystems 394 automated DNA/RNA synthesizer as reported. Cas9, crRNA-GalNAc and tracrRNA are preincubated in a 1:5:5 molar ratio in the cleavage buffer to reconstitute the Cas9::crRNA-GalNAc::tracrRNA (RNP) complex.

Alternatively, tracrRNA is prepared by in vitro transcription at the presence of a RNA polymerase such as bacteriophage T7 RNA polymerase.

### Example 3: PEGylation of CRISPR-Cas RNP Complex

The RNP (guide RNA-Cas protein) PEGylation is performed at different concentrations of m-PEG-pNP or m-PEG-NHS. RNP at 2 mg/mL in saline is incubated with varying concentrations of m-PEG-pNP or m-PEG-NHS (0.5, 1, 2, 4 and 6 mg/mL) in 1.5-mL microfuge tubes. The reaction is allowed to proceed at 30° C. for 3 hours. The PEGylated RNP complex(es) is used for in vitro DNA cleavages according to reported procedures without further purification.

Alternatively, before its storage at −80° C. or uses, size exclusion chromatography (SEC) of PEGylated RNP complex(es) is performed on a Akta Purifier using a HiLoad 16/60 S200 superdex column with gel filtration buffer (20 mM HEPES pH 7.5, 150 mM KCl, 10% (v/v) glycerol) with a flow rate of 1 mL/min. PEGylated RNP complex(es) is loaded in volumes no greater than 1 mL.

### Example 4: Expression and purification of SpCas9 (Cys-SpCas9: MIC, C80A, S145C, S204C, S469C, S1159C)

SpCas9 expression plasmid containing amino acid substitutions are generated by standard PCR and molecular cloning.

The primers are synthesized on an Applied Biosystems 394 automated DNA/RNA synthesizer using the standard 1.0 μmole DNA phosphoramidite cycle. Nucleoside on CPG support is packed into a twist column. All B-cyanoethyl phosphoramidite monomers are dissolved in anhydrous acetonitrile to a concentration of 0.1 M immediately prior to use. Stepwise coupling efficiencies are determined by automated trityl cation conductivity monitoring and in all cases are >99%.

The Cys-SpCas9 is expressed in E. coli strain Rosetta 2 DE3 cells, and the protein is isolated and purified as described (Anders, et al. Methods Enzymol. 2014, 546, 1-20).

### Example 5: PEGylation of Cys-SpCas9 Protein

PEGylation of Cys-SpCas9 protein is performed at different concentrations of m-PEG-Maleimide. Protein at 2 mg/mL in saline is incubated with varying concentrations of m-PEG-Maleimide (0.5, 1, 2, 4 and 6 mg/mL) in 1.5-mL microfuge tubes. The reaction is allowed to proceed at 30° C. for 3 hours.

Before its storage at −80° C. or uses, size exclusion chromatography (SEC) of PEGylated RNP complex(es) is performed on a Akta Purifier using a HiLoad 16/60 S200 superdex column with gel filtration buffer (20 mM HEPES pH 7.5, 150 mM KCl, 10% (v/v) glycerol) with a flow rate of 1 mL/min. PEGylated RNP complex(es) is loaded in volumes no greater than 1 mL.

### Example 6: Cys-SpCas9::lgRNA Complex

The PEGylated Cys-SpCas9 protein and lgRNA are preincubated in a 1:2 molar ratio in the cleavage buffer to reconstitute the Cas::lgRNA complex. The PEGylated RNP complex(es) is used for in vitro DNA cleavages according to reported procedures without further purification.

### Example 7: ON-1 (SEQ ID NO: 70)

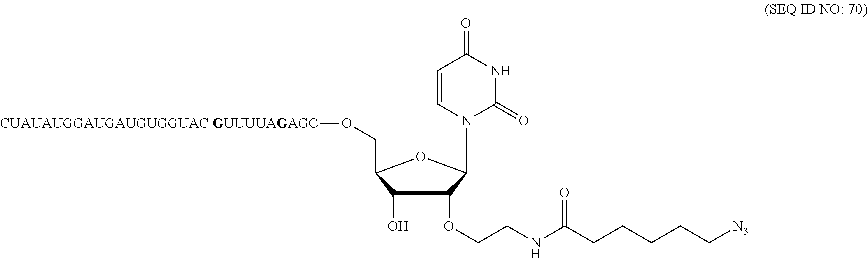

ON-1 is synthesized in a way similar to the synthesis of 3′-azide-crRNA in EXAMPLE 1, except the oligonucleotide is truncated at its 3′-end. The oligonucleotide is cleaved off the solid phase and fully deprotected, and purified as described in EXAMPLE 1.

### Example 8: ON-2

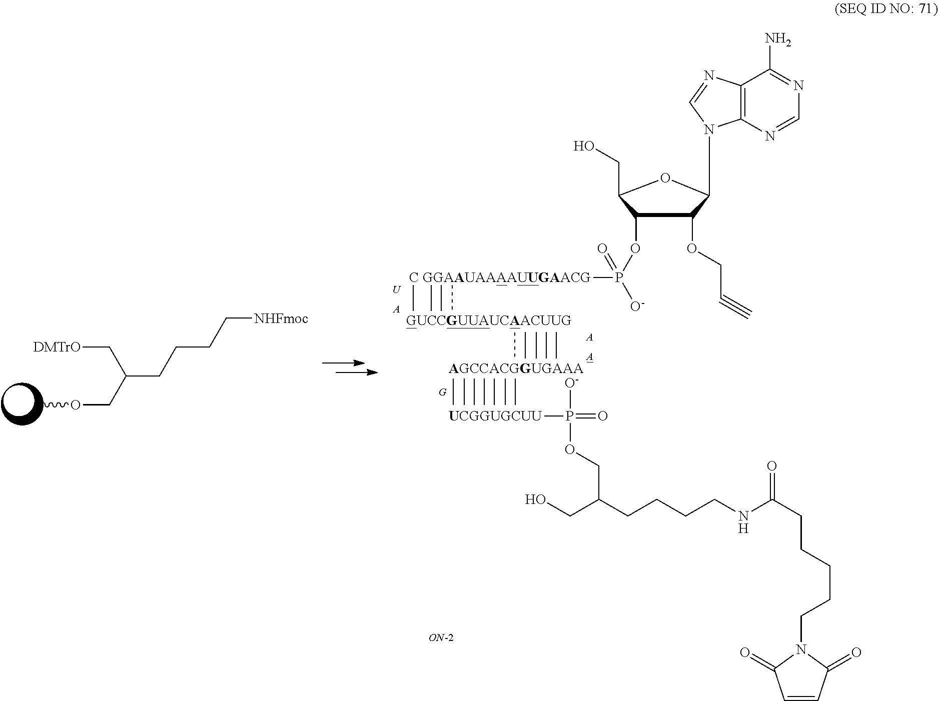

ON-2 is synthesized in a way similar to the synthesis of ON-1, except that a solid phase support linked to Fmoc-hexylamine is used, and 2′-propargyl adenosine phosphoramidite is used to introduce an alkyne.

Fmoc is then cleaved by treatment with 20% piperidine in DMF. The resulting 3′-end aminohexyl oligonucleotide is then treated with NHS ester of 6-maleimide caproic acid in DMF.

Cleavage of oligonucleotides from the solid support and full deprotection are achieved by exposure to concentrated aqueous ammonia/ethanol (3/1 v/v) for 2 h at room temperature followed by heating in a sealed tube for 45 min at 55° C. and desilylation in 1.0 M TBAF in THF for 24 h.

Alternatively, the step of reaction with NHS ester is skipped, and the above resulting fully deprotected oligonucleotide is dissolved in 0.5 M Na₂CO₃/NaHCO₃ buffer (pH 8.75) and incubated with NHS ester of 6-maleimide caproic acid (20 eq.) in DMSO to give ON-2.

### Example 9: ON-3 (SEQ ID NO: 72)

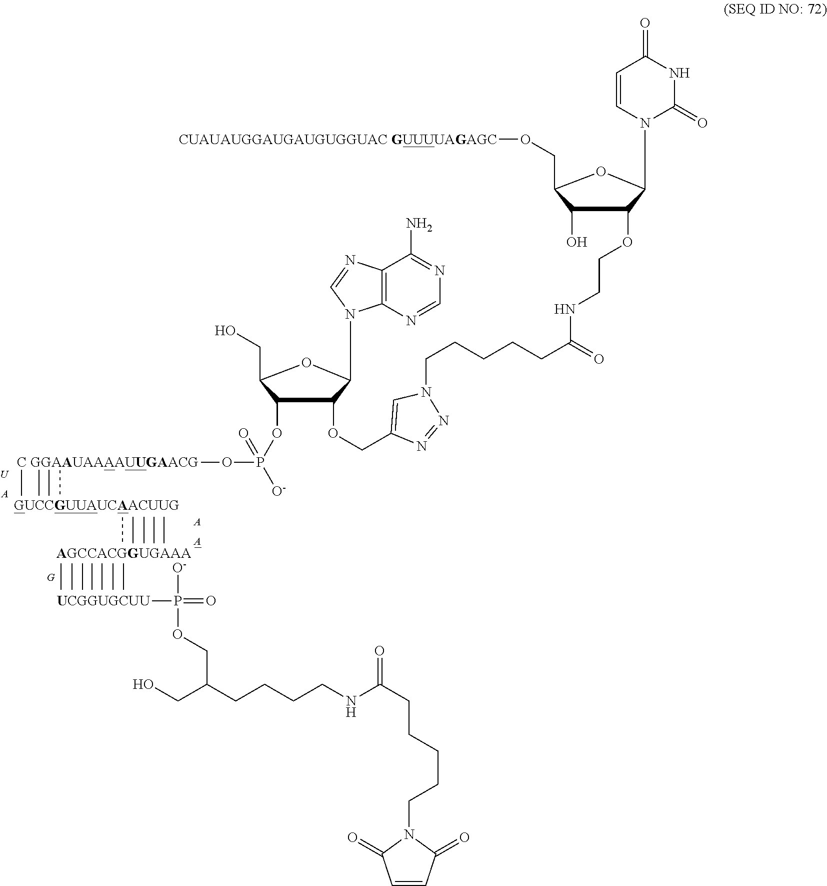

A solution of alkyne ON-1 and azide ON-2 (0.2 nmol of each) in 0.2 M NaCl (50 μL) is annealed for 30 min at room temperature. In the meantime, tris-hydroxypropyl triazole ligand (28 nmol in 42 μL 0.2 M NaCl), sodium ascorbate (40 nmol in 4 μL 0.2 M NaCl) and CuSO₄·5H₂O (4 nmol in 4.0 μL 0.2 M NaCl), are added under argon. The reaction mixture is kept under argon at room temperature for the desired time, and formamide (50 μL) is added. The reaction is analyzed by loading directly onto a 20% polyacrylamide electrophoresis gel, and purified by reversed-phase HPLC.

### Example 10: ON-4 (SEQ ID NO: 73)

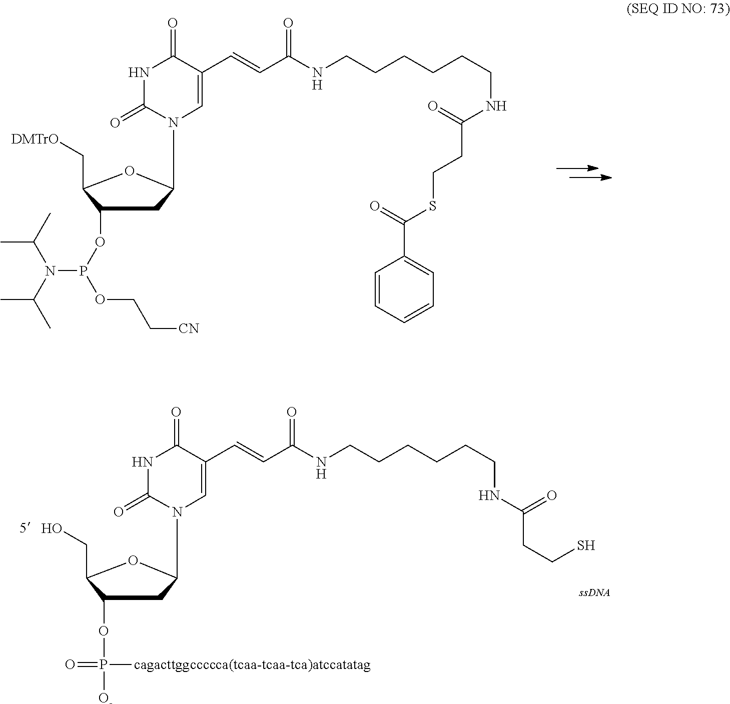

ON-4 is synthesized in a way similar to the synthesis of primers in Example 4.

Cleavage of oligonucleotides from the solid support and deprotection are achieved by exposure to concentrated aqueous ammonia/ethanol (3/1 v/v) for 2 h at room temperature followed by heating in a sealed tube for 45 min at 55° C.

### Example 11: 1gRNA (-3′, 5′-) ssDNA (SEQ ID NO: 74) and sgRNA (-3′, 5′-) ssDNA (SEQ ID NO: 75)

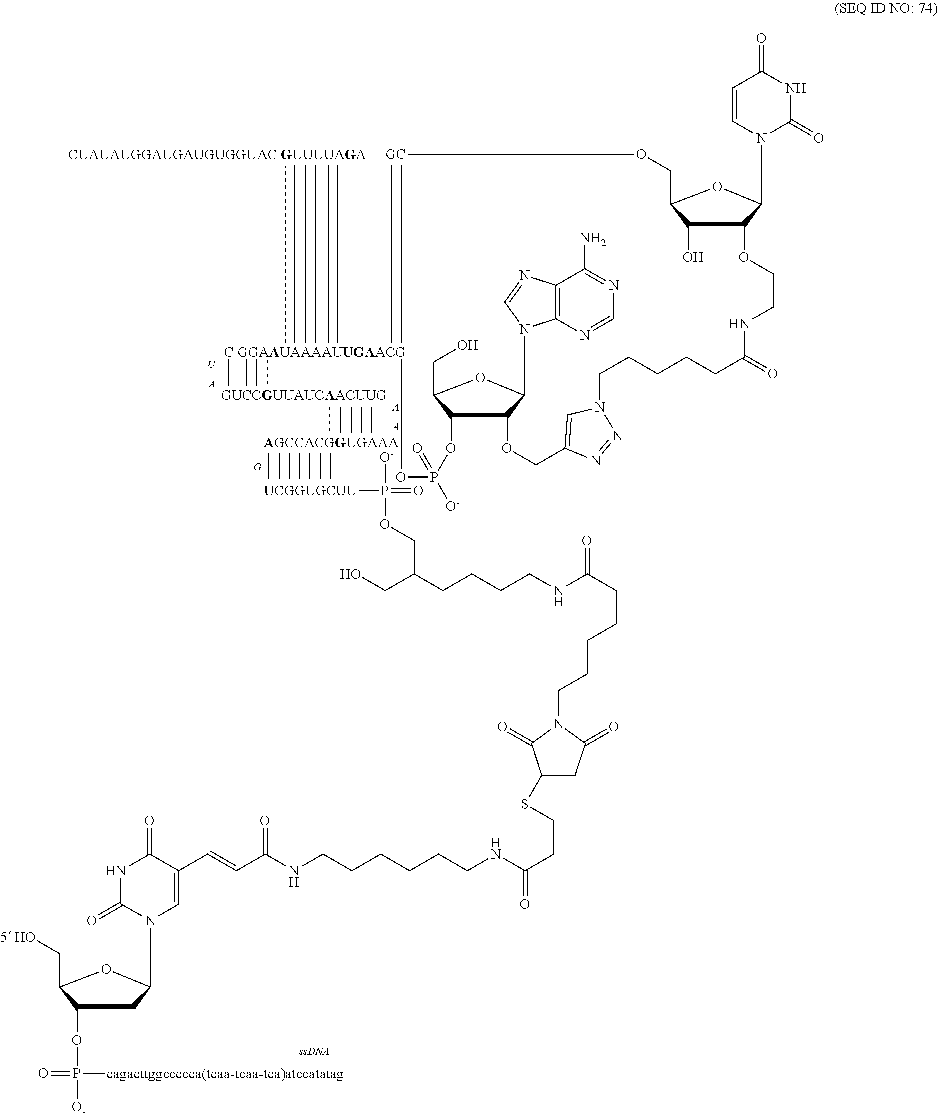

The ON-3 oligonucleotide carrying a maleimido group is incubated with 5′-SH-oligonucleotide (ON-4, 1:1 molar ratio) in 0.1 M triethylammonium acetate (TEAA) at pH 7.0 overnight at room temperature. The reaction mixture is analyzed and separated by HPLC to give the lgRNA-ssDNA.

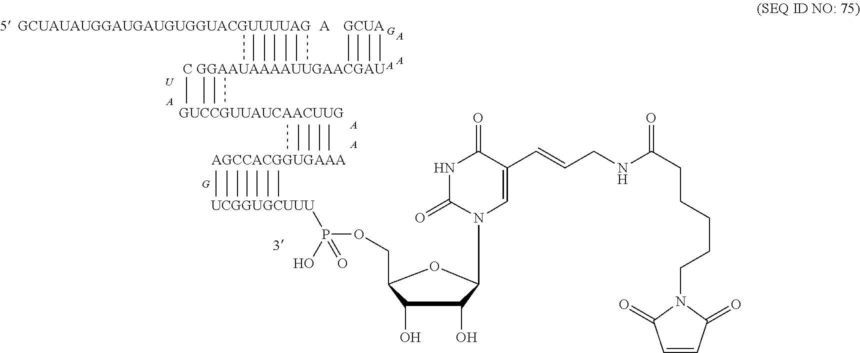

Alternatively, ON-3 oligonucleotide is substituted with an sgRNA carrying a maleimido group (ON-5 (SEQ ID NO: 75)) prepared from an sgRNA (incorporated an amine at its 3′-end) by enzymatic single nucleotide addition at the presence of a terminal deoxyribonucleotidyl transferase (TdT), followed by reacting the amine to introduce a maleimido group via NHS chemistry. The said amine at the 3′-end is incorporated from a uridine triphosphate analogue.

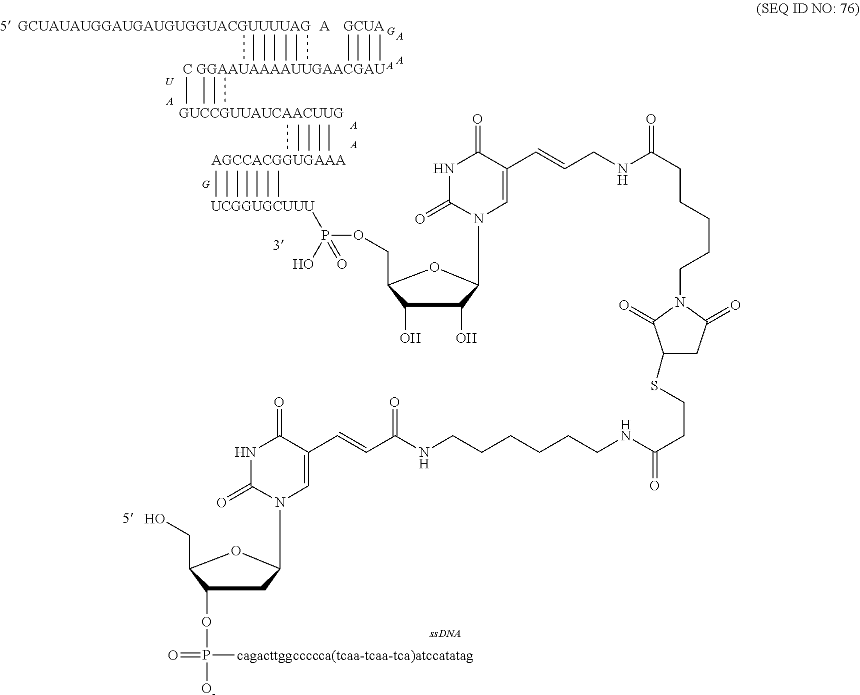

The ON-5 oligonucleotide carrying a maleimido group is incubated with 5′-SH-oligonucleotide (ON-4, 1:1 molar ratio) in 0.1 M triethylammonium acetate (TEAA) at pH 7.0 overnight at room temperature. The reaction mixture is analyzed and separated by HPLC to give the sgRNA-ssDNA.

### Example 12: ssDNA (-3′, 5′-) lgRNA (SEQ ID NO: 77)

The ssDNA oligonucleotide carrying a maleimido group at its 3′-end is incubated with the 5′-SH-lgRNA (1:1 molar ratio) in 0.1 M triethylammonium acetate (TEAA) at pH 7.0 overnight at room temperature. The reaction mixture is analyzed and separated by HPLC to give the lgRNA-ssDNA.

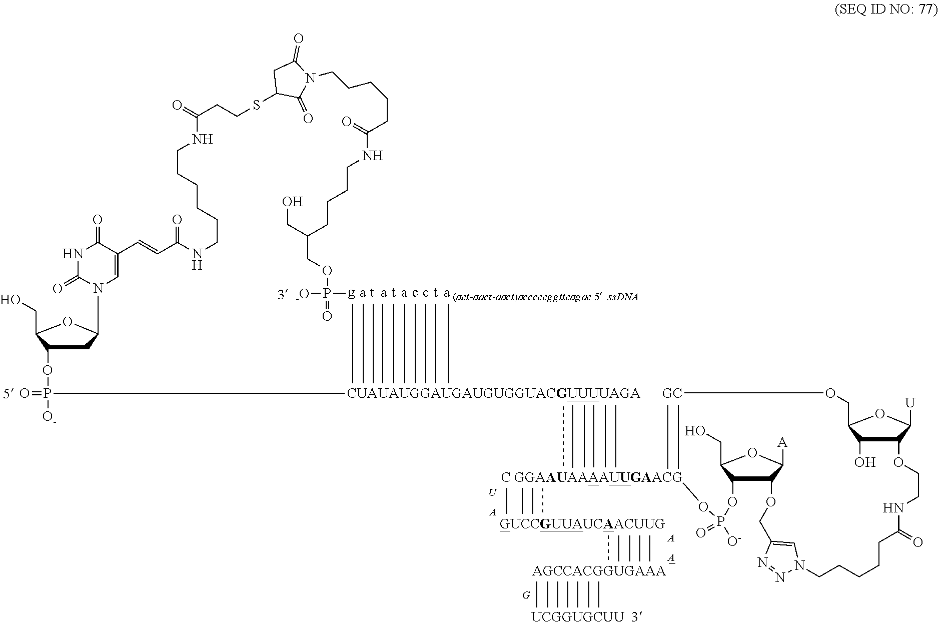

### Example 13: lgRNA (-3′, 5′-) ssDNA (SEQ ID NO: 78)

The RNA/DNA chimeric 5′-SH-tracrgRNA2-ssDNA oligonucleotide is synthesized on an Applied Biosystems 394 automated DNA/RNA synthesizer in a way similar to EXAMPLE 4 and 10. The tracrgRNA1 carrying a maleimido group at its 3′-end is synthesized in a way similar to the synthesis of ON-2 (SEQ ID NO: 71), and then ligated with ON-1 (SEQ ID NO: 70), as illustrated in EXAMPLE 9. The resulting oligonucleotide (crgRNA-tracrgRNA1) is incubated with the 5′-SH-tracrgRNA2-ssDNA (1:1 molar ratio) in 0.1 M triethylammonium acetate (TEAA) at pH 7.0 overnight at room temperature. The reaction mixture is analyzed and separated by HPLC to give the lgRNA-ssDNA.

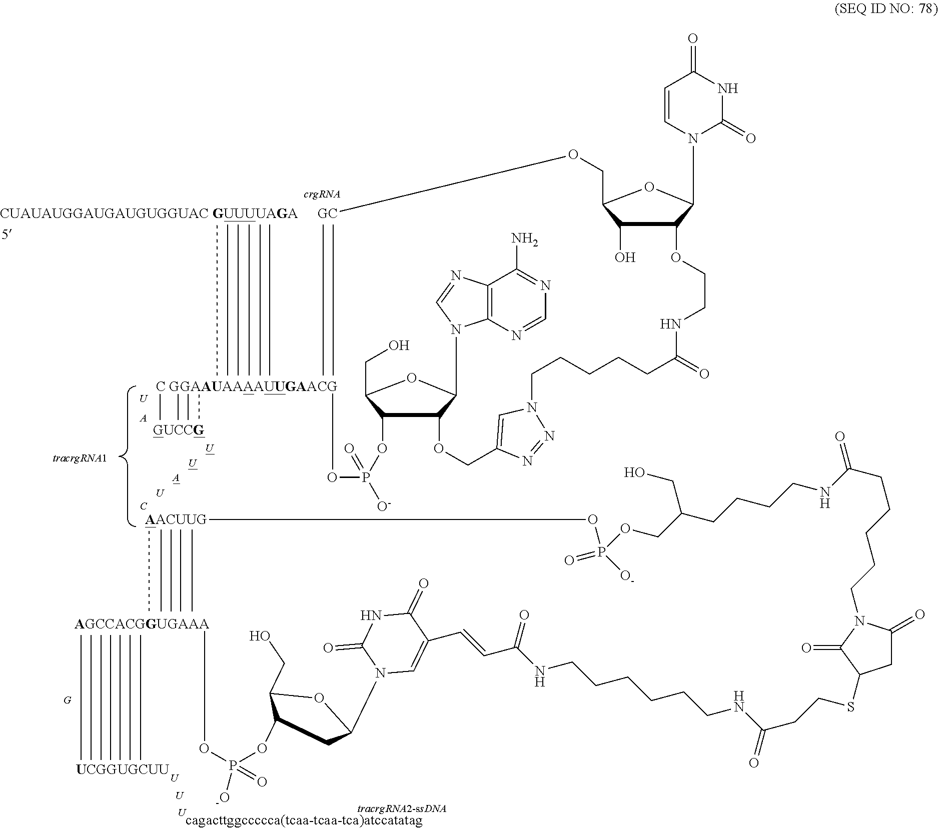

### Example 14: Cys-SpCas9/lgRNA-ssDNA Complex

The PEGylated Cys-SpCas9 protein and lgRNA-ssDNA are preincubated in a 1:2 molar ratio in the cleavage buffer to reconstitute the Cas9/lgRNA-ssDNA complex. The PEGylated RNP complex(es) is used for DNA cleavages/editing according to reported procedures without further purification.

### Example 15: In Vitro Cleavage Assay for lgRNA-ssDNA Optimization

Recombinant Cas9 protein was purchased from New England BioLabs, Inc. Cas9 and lgRNA or lgRNA-ssDNA were preincubated in a 1:1 molar ratio in the cleavage buffer to reconstitute the RNP complex.

A synthetic dsDNA was dissolved in the cleavage buffer and added to the RNP complex. The reaction mixture was incubated at 37° C. for 1 h, and DNA loading dyes (6×) was added. The resulting mixture was heated at 95° C. for 5 min, cooled to room temperature, and resolved by a 1% Agarose gel.

### Example 16: Cys-SpCas9-nickase (H840A)/lgRNA-ssDNA Complex

A Cys-SpCas9-nickase (H840A) conjugate and lgRNA-ssDNA are preincubated in a 1:2 molar ratio in the cleavage buffer to reconstitute the Cas9/lgRNA-ssDNA complex. The RNP complex(es) is used for DNA cleavages/editing according to reported procedures without further purification.

### Example 17: Cellular Transfections, and Assays of Cas9/lgRNA-ssDNA

a. Transfection with cationic lipids (Liu, D. et al. Nature Biotechnology 2015, 33, 73-80):

Purified synthetic lgRNA-ssDNA or a mixture of synthetic lgRNA-ssDNAs is incubated with purified Cas9 protein for 5 min, and then complexed with the cationic lipid reagent in OPTIMEM media. The resulting mixture is applied to the cells for 4 h at 37° C.

b. Transfection with cell-penetrating peptides (Kim, H. et al. Genome Res. 2014, 24: 1012-1019):

Cell-penetrating peptide (CPP) is conjugated to a purified recombinant Cys-Cas9 protein by dropwise mixing of 1 mg Cas9 protein (2 mg/mL) with 50 μg 4-maleimidobutyryl-GGGRRRRRRRRRLLLL (m9R; 2 mg/mL) in PBS (pH 7.4) followed by incubation on a rotator at room temperature for 2 h. To remove unconjugated m9R, the samples are dialyzed against DPBS (pH 7.4) at 4° C. for 24 h using 50 kDa molecular weight cutoff membranes. Cys-Cas9-m9R protein is collected from the dialysis membrane and the protein concentration is determined using the Bradford assay (Biorad).

Synthetic lgRNA-ssDNA or a mixture of synthetic lgRNA-ssDNAs is complexed with CPP: lgRNA-ssDNA (1 μg) in 1 μl of deionized water is gently added to the C3G9R4LC peptide (9R) in lgRNA:peptide weight ratios that range from 1:2.5 to 1:40 in 100 μl of DPBS (pH 7.4). This mixture is incubated at room temperature for 30 min and diluted 10-fold using RNase-free deionized water.

150 μl Cys-Cas9-m9R (2 μM) protein is mixed with 100 μl lgRNA-SSDNA:9R (10:50 μg) complex and the resulting mixture is applied to the cells for 4 h at 37° C. Cells can also be treated with Cys-Cas9-m9R and lgRNA-ssDNA: 9R sequentially.

### Example 18: Anti-HBV in Cells

The antiviral assay is performed according to reported procedures (Hu, W. et al. Proc Natl Acad Sci USA 2014, 110: 11461-11466; Lin, Su. et al. Molecular Therapy—Nucleic Acids, 2014, 3, e186). Delivery to cell lines is either cationic lipid or CPP based delivery of Cys-Cas9/lgRNA-ssDNA complexes instead of plasmid transfection/transduction using gRNA/Cas9 expression vectors.

Alternatively, cells are treated with lgRNA-ssDNA and mRNA encoding Cas9 protein (lgRNA-ssDNA/mRNA˜10:1) either as a mixture or sequentially in LNPs formulated with an amine-to-RNA-phosphate ratio of about 3-6 (N:P), or cells are treated with lgRNA-ssDNA in LNPs formulated with an amine-to-RNA-phosphate ratio of about 3-6 (N:P) and AAV vector encoding Cas9 protein.

### Example 19: Anti-HBV in Chimeric Mice

The antiviral assay in HBV infected chimeric mice is performed according to a reported procedure except conjugates of Cys-Cas9/lgRNA-ssDNA RNP complexes are administrated instead of small interfering RNAs (Thi, E. P. et al. ACS Infec. Dis. 2019, 5, 725-737). All animals are bred under specific pathogen-free conditions in accordance with the ethical guidelines set forth by the National Institutes of Health for care of laboratory animals. The cDNA-uPA/SCID (cDNA-uPA (+/wt)/SCID (+/+)) hemizygote mice are generated as described. Cryopreserved human hepatocytes (2-year-old female, Hispanic, BD195, BD Biosciences) are transplanted into 2-4-week-old hemizygous cDNA-uPA/SCID mice via the spleen under anesthesia. The human hepatocytes are allowed to expand for 10-12 weeks and the replacement index are tested by measuring human albumin (h-Alb) in blood collected from tail vein using clinical chemistry analyzer (BioMajesty Series JCA-BM6050, JEOL Ltd.) with latex agglutination immunonephelometry (LZ Test “Eiken” U-ALB, Eiken Chemical Co., Ltd.). Male chimeric mice with more than 7.0 mg/mL h-Alb concentration in blood are judged as PXB mice whose replacement index is more than 70%.

PXB mice (>70% replacement index, 13-15 weeks old) are infected with HBV by intravenous injection through the tail vein with 1×10⁵ copies of HBV containing serum from previously infected animals. Eight weeks post infection, animals with HBV DNA titers greater than 1.0×10⁶ copies/mL and h-Alb greater than 7.0 mg/ml are selected (n=5 per group). Cys-Cas9/lgRNA-ssDNA complexes are dosed via the lateral tail vein in a volume of 0.2 mL per animal. Animals are euthanized at various time points by exsanguination under isoflurane anesthesia. Liver tissue is collected from the median or left lateral lobe from each animal for DNA extraction and for NGS. Editing efficiency and off-targets are determined as described (Finn, J. D. et al. Cell Reports 2018, 22, 2227-2235; Tsai, S. Q. et al. Nat. Methods 2017, 14, 607-614).

Blood is collected into serum separator tubes. Serum HBV DNA is assayed by qPCR and serum HBsAg measured by chemiluminescence enzyme immunoassay (ARCHITECT, Abbott). Serum HBeAg is also assessed using a chemiluminescence enzyme immunoassay (ARCHITECT, Abbott). Liver total and 3.5 kb HBV (pg)RNA at day 42 (study termination) are analyzed by Quantigene 2.0 b DNA assay (Affymetrix), and data is normalized to human glyceraldehyde-3-phosphate dehydrogenase (GAPDH) mRNA. Immunohistochemical analysis for HBcAg is conducted on liver sections at day 42.

Alternatively, lgRNA-ssDNA and mRNA encoding Cas9 protein (lgRNA-ssDNA/mRNA˜10:1) are administrated either as a mixture or sequentially in LNPs formulated with an amine-to-RNA-phosphate ratio of about 3-6 (N:P), or AAV vector encoding Cas9 protein and lgRNA-ssDNA in LNPs formulated with an amine-to-RNA-phosphate ratio of about 3-6 (N:P) are administrated sequentially.

### Example 20: Anti-HIV in Humanized Mice

The antiviral assay in HIV infected humanized mice is performed according to a reported procedure except Cys-Cas9/lgRNA-ssDNA RNP complexes are administrated instead of the AAV9-CRISPR-Cas9 vector (Dash, P. K. et al. Nat. Comm. 2019, 10, 1-20).

CD34+HSC are enriched from human cord blood or fetal liver cells using immune-magnetic beads. CD34+cell purity was >90% by flow cytometry. Cells are transplanted into newborn mice irradiated at 1 Gy using a RS-2000 X-Ray Irradiator by intrahepatic (i.h.) injection of 50,000 cells/mouse in 20 μl phosphate-buffered saline (PBS) with a 30-gauge needle. At 18 weeks of age, NSG-hu mice are infected intraperitoneally (i.p.) with HIV-1_{NL4-3} at 10⁴ tissue culture infective dose₅₀ (TCID₅₀)/ml.

Cys-Cas9/lgRNA-ssDNA complexes are dosed via the lateral tail vein in a volume of 0.2 mL per animal. HIV-1 nucleic acids are detected by ddPCR and editing efficiency and off-targets are determined as described.

Alternatively, lgRNA-ssDNA and mRNA encoding Cas9 protein (ssDNA-lgRNA/mRNA˜10:1) are administrated either as a mixture or sequentially in LNPs formulated with an amine-to-RNA-phosphate ratio of about 3-6 (N:P), or AAV vector encoding Cas9 protein and lgRNA-ssDNA in LNPs formulated with an amine-to-RNA-phosphate ratio of about 3-6 (N:P) are administrated sequentially.

### Example 21: AAV-EFS_CMV::NLS-SaCas9-NLS-polyA;U6-tracrRNA Plasmid

5'-caccact tatac ctaaa attac agaat ctact aaaac
aaggc aaaat gccgt gttta tctcg tcaac ttgtt ggcga
gattt tt-3' (top, SEQ ID NO: 79)
5'-ggccaa aaatc tcgcc aacaa gttga cgaga taaac
acggc atttt gcctt gtttt agtag attct gtaat tttag
gtata agt-3' (bottom, SEQ ID NO: 80)

The top and bottom strands of oligonucleotides for cDNA encoding tracrRNA of SaCas9 system are synthesized using DNA phosphoramidite monomers with routine protection of the A, G and C nucleobases and unprotected thymine. 0.45 M tetrazole in acetonitrile is used as the coupling agent, acetic anhydride as the capping agent and 0.1 M iodine as the oxidizing agent. Oligonucleotide synthesis is carried out on an Applied Biosystems 394 automated DNA/RNA synthesizer using the standard 1.0 μmole DNA phosphoramidite cycle. Nucleoside on CPG support is packed into a twist column. All β-cyanoethyl phosphoramidite monomers are dissolved in anhydrous acetonitrile to a concentration of 0.1 M immediately prior to use. Stepwise coupling efficiencies are determined by automated trityl cation conductivity monitoring and in all cases are >99%.

Viral plasmids are produced utilizing standard recombinant DNA cloning techniques.

The above cDNA strands (1:1) are phosphorylated and annealed, and cloned into a linearized pX601-AAV-EFS_CMV::NLS-SaCas9-NLS-polyA3×HA-bGHpA;U 6::BsaI-sgRNA expression plasmid vector (Addgene gene plasmid #61591) as following. The AAV vector is digested with BsaI and NotI to remove the insert of sgRNA scaffold, treated with antarctic phosphatase, and purified with a Quick nucleotide removal kit (QIAGEN). An equal amount of complementary oligonucleotide is mixed in T4 polynucleotide kinase (PNK) buffer for annealing. These annealed seed pairs are phosphorylated with T4 PNK and ligated into the BsaI- and NotI digested AAV using T7 DNA ligase.

### Example 22: Virus Production

The AAV::SaCas9 virus is packaged using a triple plasmid transfection method as described (Chew, et al. Nature Methods, 2016). 293 FT cells (Life Technologies) are plated in growth media consisting of DMEM+glutaMAX+pyruvate+10% FBS (Life Technologies), supplemented with 1× MEM non-essential amino acids (Gibco) in 150 mm plates. Confluency at transfection is between 70-90%. 20 μg of pHelper plasmid, 10 μg of pRepCap plasmid (encoding capsid proteins), and 10 μg of AAV plasmid carrying the construct of interest are mixed in 500 μL of DMEM, and 200 μg of PEI “MAX” (Polysciences) (40 kDa, 1 mg/mL in H₂O, pH 7.1) are added for PEI:DNA mass ratio of 5:1. The mixture is incubated for 15 minutes, and transferred dropwise to the cell media. The day after transfection, media is changed to DMEM+glutamax+pyruvate+2% FBS. Cells are harvested 48-72 hours after transfection by scrapping or dissociation with 1×PBS (pH7.2)+5 mM EDTA, and pelleted at 1500 g for 12 min. Cell pellets are resuspended in 1-5 mL of lysis buffer (Tris HCl pH 7.5+2 mM MgCl2+150 mM NaCl), and freeze-thawed 3× between dry-ice-ethanol bath and 37° C. water bath. Cell debris is clarified via 4000 g for 5 minutes, and the supernatant is collected.

The collected AAV supernatant is treated with 50 U/mL Benzonase and 1 U/mL Riboshredder for 30 minutes at 37° C. After incubation, the lysate is concentrated to <3 mL by ultrafiltration with Amicon Ultra-15 (50 kDa MWCO) (Millipore), and loaded on top of a discontinuous density gradient consisting of 2 mL each of 15%, 25%, 40%, 60% Optiprep (Sigma-Aldrich) in an 11.2 mL Optiscal polypropylene tube (Beckman-Coulter). The tubes are ultracentrifuged at 58000 rpm, at 18° C., for 1.5 hours, on an NVT65 rotor. The 40% fraction is extracted, and dialyzed with 1×PBS (pH 7.2) supplemented with 35 mM NaCl, using Amicon Ultra-15 (50 kDa or 100 kDa MWCO) (Millipore). The purified AAVs are stored at −80° C. as 25 μL aliquots.

AAV titers (vector genomes) are quantified via hydrolysis probe qPCR against standard curves generated from linearized parental AAV plasmids.

### Example 23: Generation of Cas9-tracrRNA Stable Cells and Transfection With crRNA(s)

Cells are plated at 2×10⁴ per well in a 96-well plate in 100 μL of growth media. Purified AAVs are applied at confluency of 70-90%. Culture media is replaced with fresh growth media the next day, and the cells are incubated at 37° C. and 5% CO₂ for 24 hours. Culture media is replaced with fresh media, and the cells are transfected with crRNAs either gymnotically or with transfection reagents.

### Example 24: Mice Injection

AAV vector and crRNAs are delivered to 5-6 week old male mice intravenously via lateral tail vein injection. All dosages of AAV are adjusted to 100 μL or 200 μL with sterile phosphate buffered saline (PBS), pH 7.4 (Gibco) before the injection.

### Example 25: Generation of Inducible Cas9 Stable Cells and Transfection With lgRNA(s)

Cells are transduced with Edit-R Inducible Lentiviral hEF1α-Blast-Cas9 Nuclease Particles as detailed in the manufacturer's protocol. Cells are plated at 5×10⁴ cells per well in a 24-well plate using Tet-free growth medium, incubated at 37° C. in a humidified CO₂-incubator overnight, and transduced with Edit-R Inducible Lentiviral hEF1α-Blast-Cas9 Nuclease. The incubation is continued for 4-6 hours, and the medium is replaced with Tet-free growth medium and the incubation is continued for 24-48 hours. Inducible Cas9 stable cells are selected in the medium with Tet-free selection medium containing the appropriate amount of blasticidin and expanded.

The selected inducible Cas9 stable cells are then induced in freshly prepared doxycycline solution for at least 24 hours. The cells are transfected using Lipofectamine RNAiMax and the lgRNA(s). The medium is replaced with fresh medium after 12 hours. Cells are then grown for 72 hours with the media replaced when necessary.

### EXAMPLE 26: Synthesis of lgRNA-m5C-ssDNA for Epigenome Editing by a STAR Editor

LgRNA-m5C-ssDNA is synthesized similarly to the sgRNA (-3′, 5′-) ssDNA (SEQ ID NO: 75). m5C-ssDNA installed with 5-methyl cytosines at specific position(s) is synthesized and used instead. 5-Methyl cytosine is introduced into ssDNA using 5′-dimethoxytrityl-N-acetyl-5-methyl-2′-deoxycytidine,3′- [(2-cyanoethyl)-(N,N-diisopropyl)]-phosphoramidite. The lgRNA-m5C-ssDNA product is synthesized by chemical ligations of its shortmers, separated and purified as before.

### Example 27: DNA Methylation of Genomic DNA

Cells are transfected with LgRNA-m5C-ssDNA similarly to EXAMPLE 16. Genomic DNA is extracted and sequenced according to a reported procedure by whole genome bisulfite sequencing (WGBS) and analysis (Swain, T. and et al. Nucleic Acids Research, 2024, 52, 474-491).

### Equivalents

The present disclosure is not to be limited in terms of the particular embodiments described in this application, which are intended as single illustrations of individual aspects of the present disclosure. Many modifications and variations of this present disclosure can be made without departing from its spirit and scope, as will be apparent to those skilled in the art. Functionally equivalent methods and apparatuses within the scope of the present disclosure, in addition to those enumerated herein, will be apparent to those skilled in the art from the foregoing descriptions. Such modifications and variations are intended to fall within the scope of the present disclosure. It is to be understood that this present disclosure is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 or 1 to 3 items refers to groups having 1, 2, or 3 items. Similarly, a group having 1-5 or 1 to 5 items refers to groups having 1, 2, 3, 4, or 5 items, and so forth.

All patents, patent applications, provisional applications, and publications referred to or cited herein are incorporated by reference in their entirety, including all figures and tables, to the extent they are not inconsistent with the explicit teachings of this specification.

DNA sequence of Cys-SpCas9 protein:

(SEQ ID NO: 81)
tgcgacaagaagtacagcatcggcctggacatcggcaccaactctgtggg
ctgggccgtgatcaccgacgagtacaaggtgcccagcaagaaattcaagg
tgctgggcaacaccgaccggcacagcatcaagaagaacctgatcggagcc
ctgctgttcgacagcggcgaaacagccgaggccacccggctgaagagaac
cgccagaagaagatacaccagacggaagaaccggatcgcctatctgcaag
agatcttcagcaacgagatggccaaggtggacgacagcttcttccacaga
ctggaagagtccttcctggtggaagaggataagaagcacgagcggcaccc
catcttcggcaacatcgtggacgaggtggcctaccacgagaagtacccca
ccatctaccacctgagaaagaaactggtggactgcaccgacaaggccgac
ctgcggctgatctatctggccctggcccacatgatcaagttccggggcca
cttcctgatcgagggcgacctgaaccccgacaacagcgacgtggacaagc
tgttcatccagctggtgcagacctacaaccagctgttcgaggaaaacccc
atcaacgcctgcggcgtggacgccaaggccatcctgtctgccagactgag
caagagcagacggctggaaaatctgatcgcccagctgcccggcgagaaga
agaatggcctgttcggaaacctgattgccctgagcctgggcctgaccccc
aacttcaagagcaacttcgacctggccgaggatgccaaactgcagctgag
caaggacacctacgacgacgacctggacaacctgctggcccagatcggcg
accagtacgccgacctgtttctggccgccaagaacctgtccgacgccatc
ctgctgagcgacatcctgagagtgaacaccgagatcaccaaggcccccct
gagcgcctctatgatcaagagatacgacgagcaccaccaggacctgaccc
tgctgaaagctctcgtgcggcagcagctgcctgagaagtacaaagagatt
ttcttcgaccagagcaagaacggctacgccggctacattgacggcggagc
cagccaggaagagttctacaagttcatcaagcccatcctggaaaagatgg
acggcaccgaggaactgctcgtgaagctgaacagagaggacctgctgcgg
aagcagcggaccttcgacaacggcagcatcccccaccagatccacctggg
agagctgcacgccattctgcggcggcaggaagatttttacccattcctga
aggacaaccgggaaaagatcgagaagatcctgaccttccgcatcccctac
tacgtgggccctctggccaggggaaacagcagattcgcctggatgaccag
aaagtgcgaggaaaccatcaccccctggaacttcgaggaagtggtggaca
agggcgcttccgcccagagcttcatcgagcggatgaccaacttcgataag
aacctgcccaacgagaaggtgctgcccaagcacagcctgctgtacgagta
cttcaccgtgtataacgagctgaccaaagtgaaatacgtgaccgagggaa
tgagaaagcccgccttcctgagcggcgagcagaaaaaggccatcgtggac
ctgctgttcaagaccaaccggaaagtgaccgtgaagcagctgaaagagga
ctacttcaagaaaatcgagtgcttcgactccgtggaaatctccggcgtgg
aagatcggttcaacgcctccctgggcacataccacgatctgctgaaaatt
atcaaggacaaggacttcctggacaatgaggaaaacgaggacattctgga
agatatcgtgctgaccctgacactgtttgaggacagagagatgatcgagg
aacggctgaaaacctatgcccacctgttcgacgacaaagtgatgaagcag
ctgaagcggcggagatacaccggctggggcaggctgagccggaagctgat
caacggcatccgggacaagcagtccggcaagacaatcctggatttcctga
agtccgacggcttcgccaacagaaacttcatgcagctgatccacgacgac
agcctgacctttaaagaggacatccagaaagcccaggtgtccggccaggg
cgatagcctgcacgagcacattgccaatctggccggcagccccgccatta
agaagggcatcctgcagacagtgaaggtggtggacgagctcgtgaaagtg
atgggccggcacaagcccgagaacatcgtgatcgaaatggccagagagaa
ccagaccacccagaagggacagaagaacagccgcgagagaatgaagcgga
tcgaagagggcatcaaagagctgggcagccagatcctgaaagaacacccc
gtggaaaacacccagctgcagaacgagaagctgtacctgtactacctgca
gaatgggcgggatatgtacgtggaccaggaactggacatcaaccggctgt
ccgactacgatgtggaccatatcgtgcctcagagctttctgaaggacgac
tccatcgacaacaaggtgctgaccagaagcgacaagaaccggggcaagag
cgacaacgtgccctccgaagaggtcgtgaagaagatgaagaactactggc
ggcagctgctgaacgccaagctgattacccagagaaagttcgacaatctg
accaaggccgagagaggcggcctgagcgaactggataaggccggcttcat
caagagacagctggtggaaacccggcagatcacaaagcacgtggcacaga
tcctggactcccggatgaacactaagtacgacgagaatgacaagctgatc
cgggaagtgaaagtgatcaccctgaagtccaagctggtgtccgatttccg
gaaggatttccagttttacaaagtgcgcgagatcaacaactaccaccacg
cccacgacgcctacctgaacgccgtcgtgggaaccgccctgatcaaaaag
taccctaagctggaaagcgagttcgtgtacggcgactacaaggtgtacga
cgtgcggaagatgatcgccaagagcgagcaggaaatcggcaaggctaccg
ccaagtacttcttctacagcaacatcatgaactttttcaagaccgagatt
accctggccaacggcgagatccggaagcggcctctgatcgagacaaacgg
cgaaaccggggagatcgtgtgggataagggccgggattttgccaccgtgc
ggaaagtgctgagcatgccccaagtgaatatcgtgaaaaagaccgaggtg
cagacaggcggcttcagcaaagagtctatcctgcccaagaggaacagcga
taagctgatcgccagaaagaaggactgggaccctaagaagtacggcggct
tcgacagccccaccgtggcctattctgtgctggtggtggccaaagtggaa
aagggcaagtccaagaaactgaagtgcgtgaaagagctgctggggatcac
catcatggaaagaagcagcttcgagaagaatcccatcgactttctggaag
ccaagggctacaaagaagtgaaaaaggacctgatcatcaagctgcctaag
tactccctgttcgagctggaaaacggccggaagagaatgctggcctctgc
cggcgaactgcagaagggaaacgaactggccctgccctccaaatatgtga
acttcctgtacctggccagccactatgagaagctgaagggctcccccgag
gataatgagcagaaacagctgtttgtggaacagcacaagcactacctgga
cgagatcatcgagcagatcagcgagttctccaagagagtgatcctggccg
acgctaatctggacaaagtgctgtccgcctacaacaagcaccgggataag
cccatcagagagcaggccgagaatatcatccacctgtttaccctgaccaa
tctgggagcccctgccgccttcaagtactttgacaccaccatcgaccgga
agaggtacaccagcaccaaagaggtgctggacgccaccctgatccaccag
agcatcaccggcctgtacgagacacggatcgacctgtctcagctgggagg
cgac

Sequence of Cys-SpCas9-NLS protein:

(SEQ ID NO: 82)
CDKKYSIGLDIGTNSVGWAVITDEYKVPSKKFKVLGNTDRHSIKKNLIGA
LLFDSGETAEATRLKRTARRRYTRRKNRIAYLQEIFSNEMAKVDDSFFHR
LEESFLVEEDKKHERHPIFGNIVDEVAYHEKYPTIYHLRKKLVCSTDKAD
LRLIYLALAHMIKFRGHFLIEGDLNPDNSDVDKLFIQLVQTYNQLFEENP
INACGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKCEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKCVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGDAYPYDVPDYASLGSGSPKKKRKVD

What is claimed is:

1. A conjugate of CRISPR-Cas protein-guide RNA(s) complex, comprising
a. a gRNA-Cas protein (RNP) complex and
b. a DNA template chemically linked to said gRNA comprising at least one 5-methylcytosine to form a gRNA-DNA conjugate.

2. Said gRNA-DNA conjugate of claim 1 covalently linked with one or more molecules selected from the group consisting of PEGs, non-PEG polymers, ligands of cellular receptors, lipids, oligonucleotides, polysaccharides, glycans, peptides, aptamers and/or antibodies to form a gRNA conjugate, and the said more molecules can be the same or different.

3. Said conjugate of CRISPR-Cas protein-guide RNA(s) complex of claim 1, wherein said Cas protein is covalently linked with one or more molecules selected from the group consisting of PEG, non-PEG polymers, ligands of cellular receptors, lipids, oligonucleotides, antibodies, polysaccharides, aptamers, glycans and peptides to form a Cas protein conjugate, and the said more molecules can be the same or different.

4. Said gRNA-DNA conjugate of claim 2 comprising absent, one or more nucleotides modified at sugar moieties selected from the group consisting of: 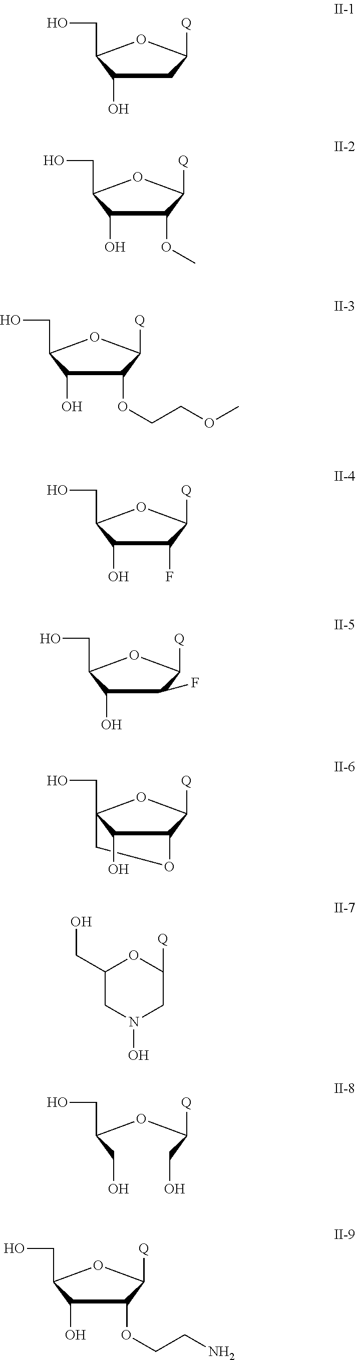 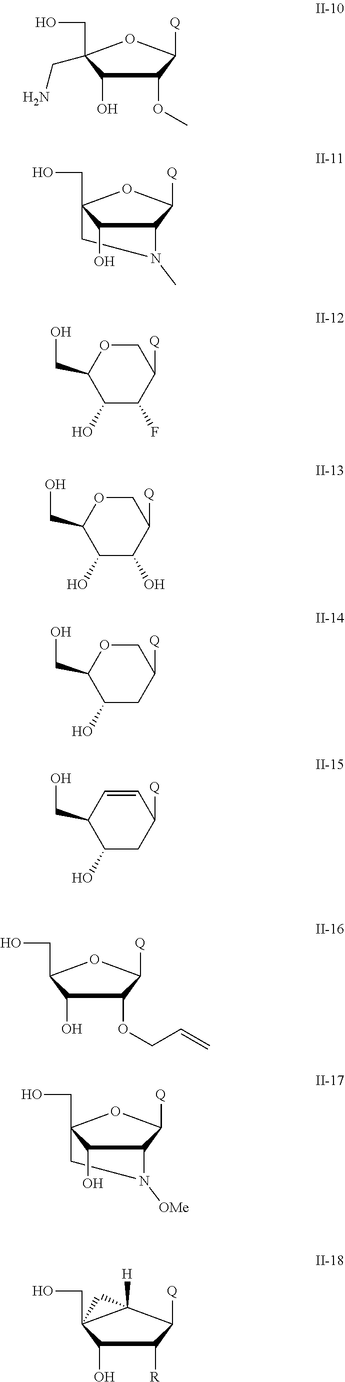 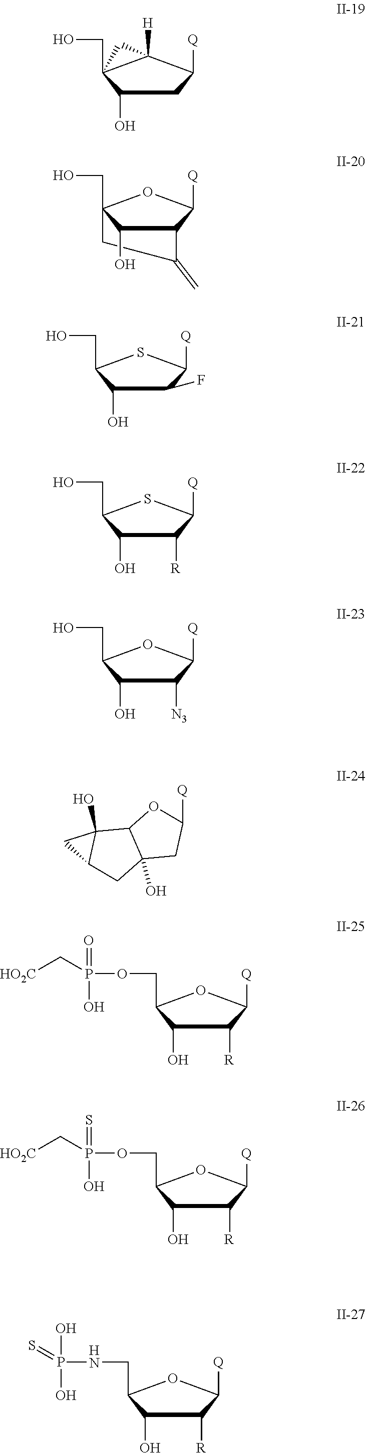 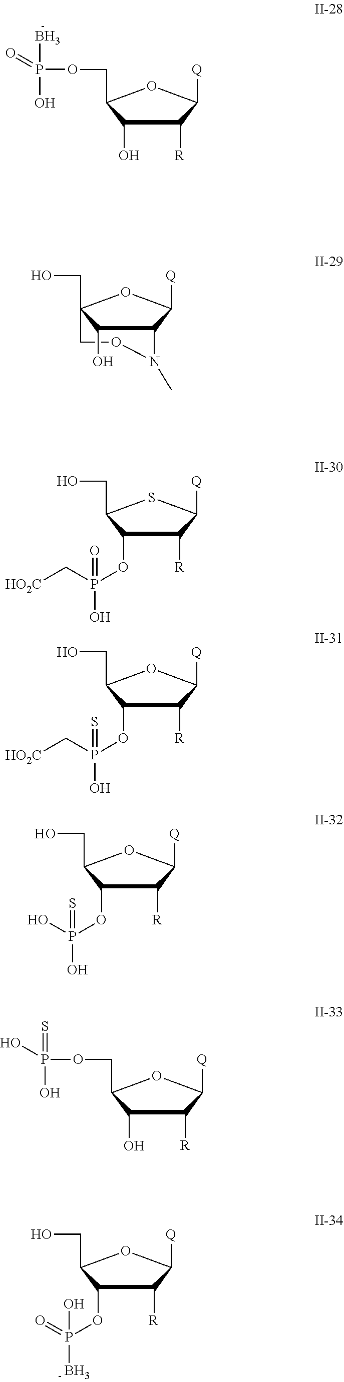 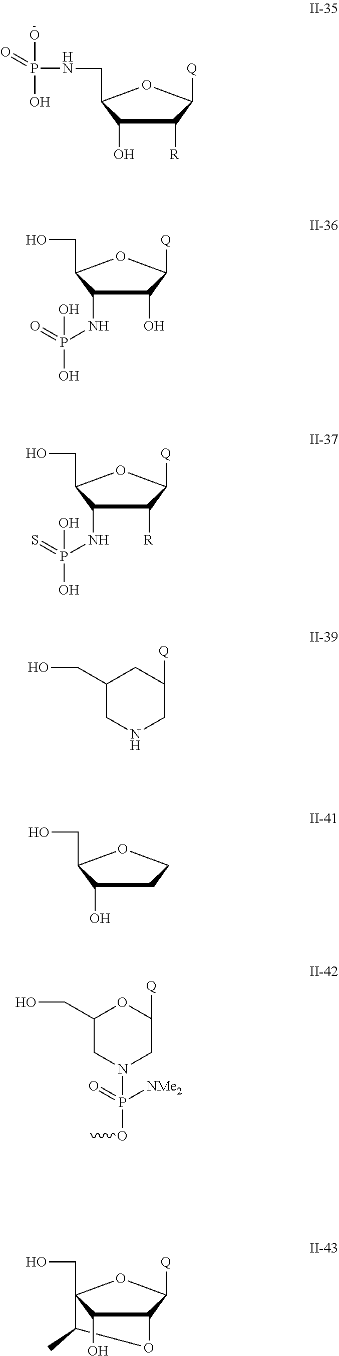
wherein R is H, OH, F, OMe, or OCH₂CH₂OCH₃ and Q is an optionally modified nucleobase, and said nucleobases are selected from the group consisting of: 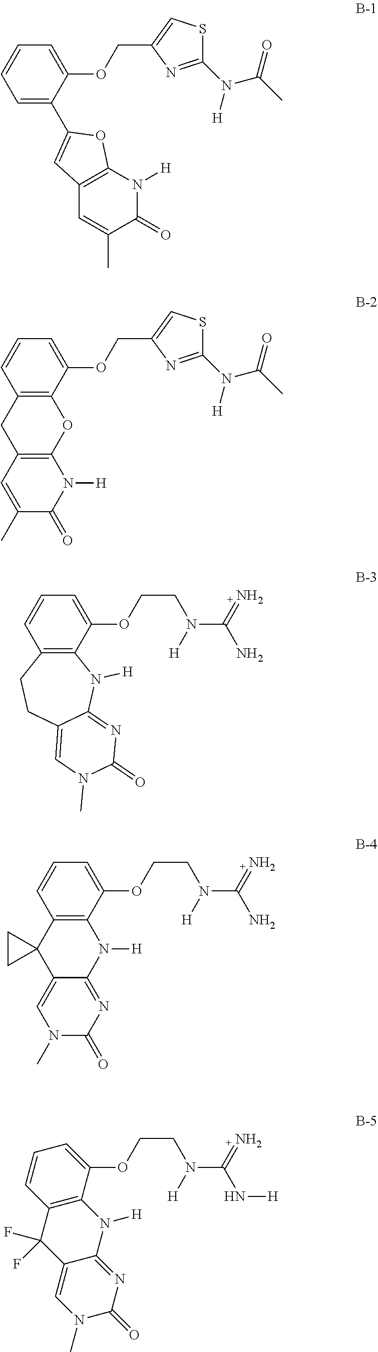 [SMILES: CC(=O)Nc1nc(COc2ccccc2-c2cc3cc(C)c(=O)[nH]c3o2)cs1 ; CC(=O)Nc1nc(COc2cccc3c2Oc2[nH]c(=O)c(C)cc2C3)cs1 ; Cn1cc2c(nc1=O)Nc1c(cccc1OCCNC(=N)N)CC2 ; Cn1cc2c(nc1=O)Nc1c(OCCNC(=N)N)cccc1C21CC1 ; Cn1cc2c(nc1=O)Nc1c(OCCNC(=N)N)cccc1C2(F)F] 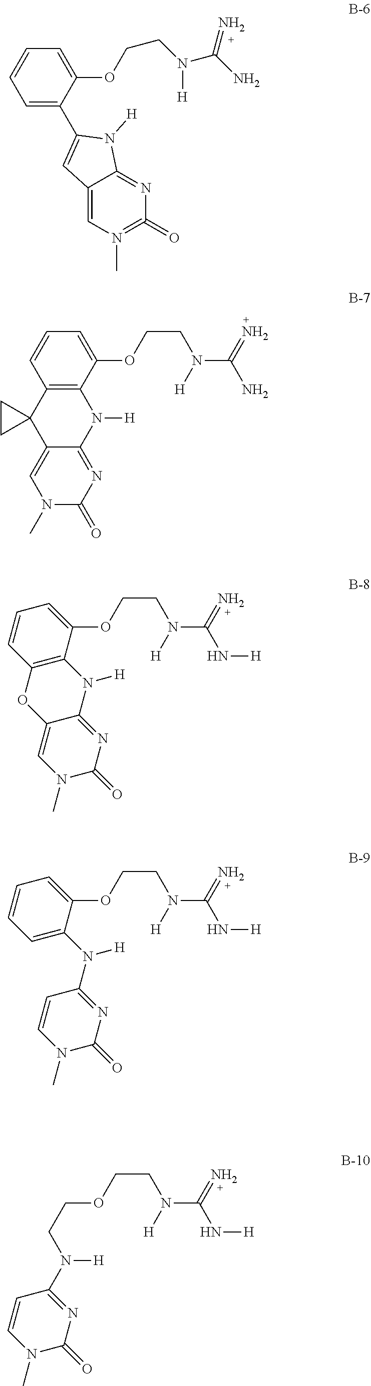 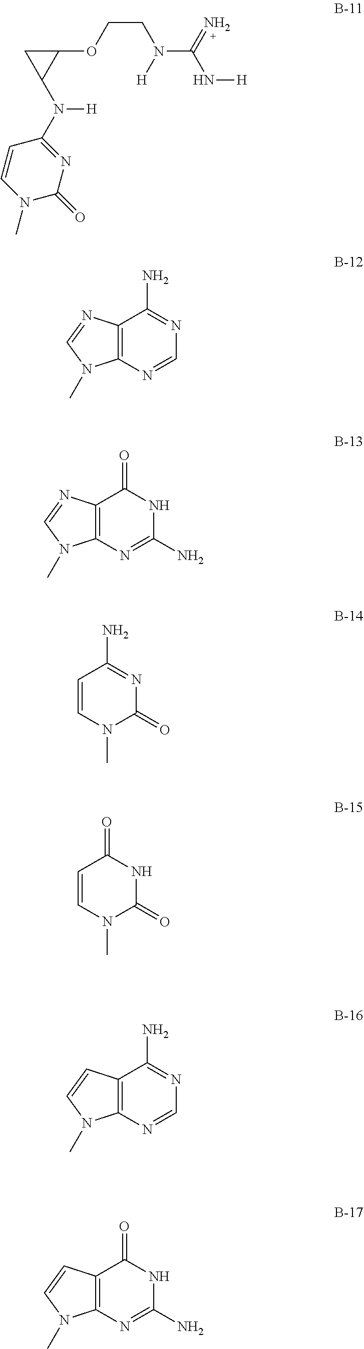 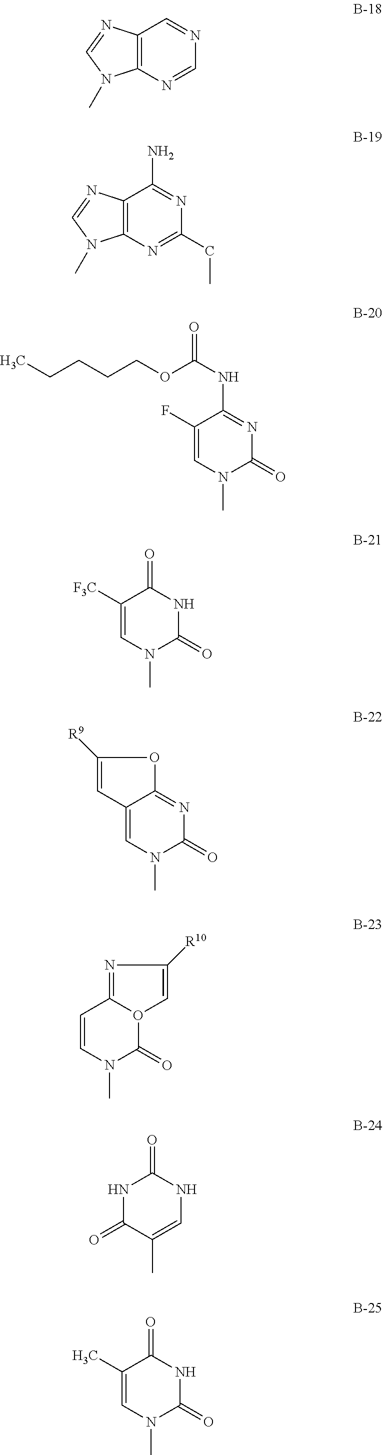 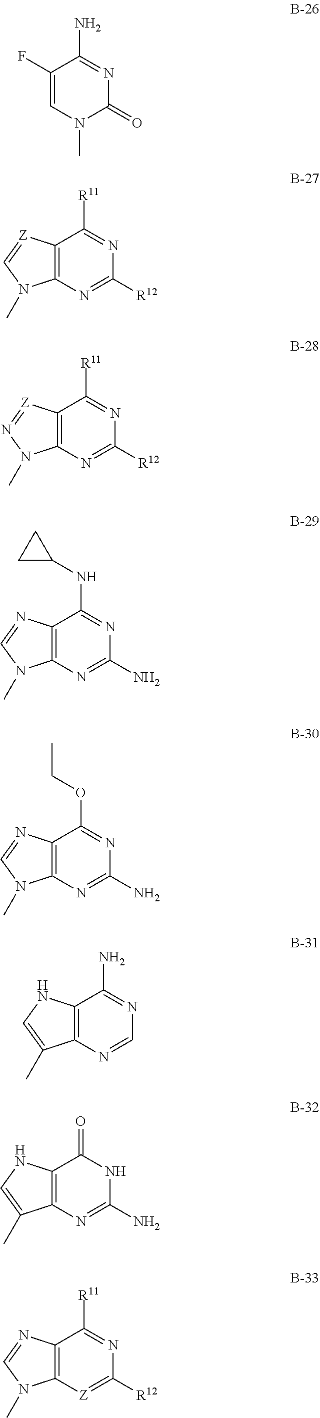 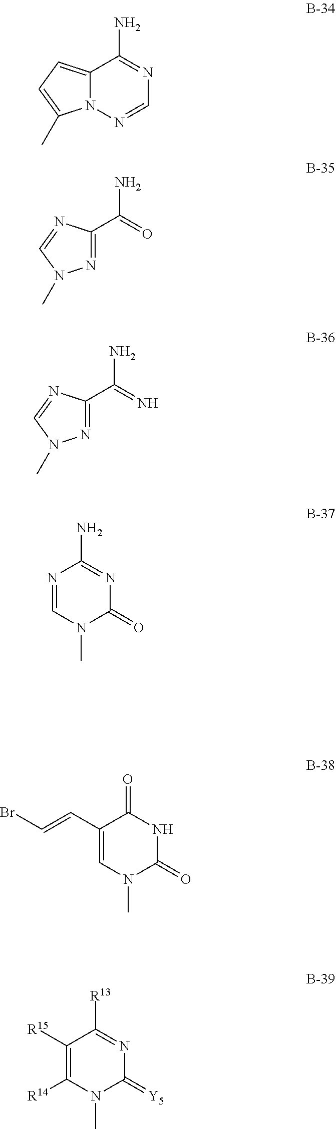 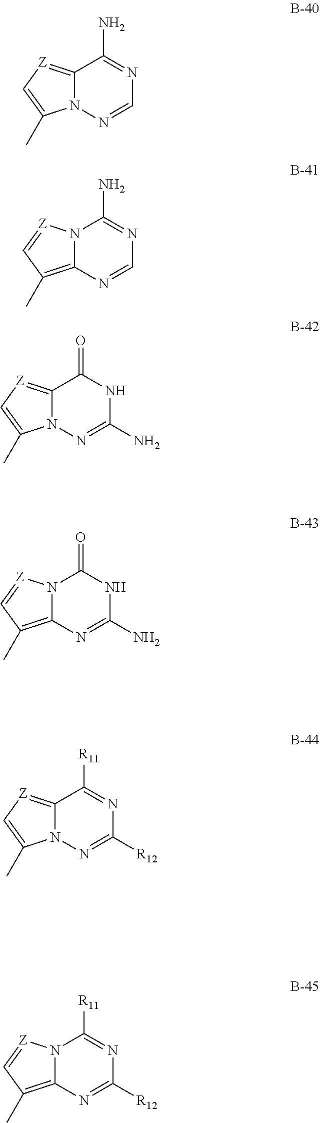
wherein:
i. Z is N or CR¹⁶;
ii. R⁹, R¹⁰, R¹¹, R¹², R¹³, R¹⁴, R¹⁵ and R¹⁶ are independently H, F, Cl, Br, I, OH, OR′, SH, SR′, SeH, SeR′, NH₂, NHR′, NHOH, NHOR′, NR′OR′, NR′₂, NHNH₂, NR′NH₂, NR′NHR′, NHNR′₂, NR′NR′₂, lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, CN, CO₂H, CO₂R′, CONH₂, CONHR′, CONR′₂, CH═CHCO₂H, or CH═CHCO₂R′, wherein R′is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

5. Said gRNA-DNA conjugate of claim 2 comprising a spacer selected from sequences of 12˜20 nt in HIV genomes, of which each thymine is replaced with uracil.

6. Said gRNA-DNA conjugate of claim 2 comprising a spacer selected from sequences of 12˜20 nt in HBV genomes, of which each thymine is replaced with uracil.

7. Said gRNA-DNA conjugate of claim 2 comprising a spacer selected from sequences of 12˜20 nt in HSV genomes, of which each thymine is replaced with uracil.

8. Said gRNA-DNA conjugate of claim 2 comprising a spacer selected from sequences of 12˜20 nt in EBV genomes, of which each thymine is replaced with uracil.

9. Said gRNA-DNA conjugate of claim 2 comprising a spacer selected from sequences of 12˜20 nt of a host genome to be edited, of which each thymine is replaced with uracil.

10. Said gRNA-DNA conjugate of claim 2 comprising a gRNA and one or two conjugated ssDNA templates, which comprises two sequences overlapping with the target strand or with the non-target strand of the DNA duplex to be edited and a gene editing sequence, and the said two sequences flanking the said gene editing sequence are optionally chemically modified, wherein said conjugation is by a non-nucleotide linker or an oligonucleotide linker.

11. Said gRNA-DNA conjugate of claim 10, wherein said gene editing sequence comprises an oligonucleotide sequence to introduce insertion(s) of one or more transcription cis-regulatory elements, and said more elements comprises repetitive sequence separated by absent or more nucleotides in between or different sequences separated by absent or more nucleotides in between.

12. Said gRNA-DNA conjugate of claim 10, wherein the 3′-homology arm of said DNA template comprises an optionally chemically modified DNA or chimeric DNA/RNA oligonucleotide segment of 7 to 14 nucleotides complementary to the PAM distal 5′-end of the spacer sequence of gRNA and next to a non-complementary gene editing sequence at the 5′-end of said segment to minimize the self-inhibition.

13. Said conjugate of CRISPR-Cas protein-guide RNA(s) complex of claim 1 comprising a mixture of gRNAs or gRNA conjugates of various spacers targeting at different loci of target genomes, and/or sequences correlated with drug resistance variants or viral quasispecies of a single locus of target genomes.

14. Said conjugate of CRISPR-Cas protein-guide RNA(s) complex of claim 1, wherein said Cas protein is a recombinant engineered class 2 endonuclease, deactivated class 2 Cas endonuclease, nickase, or Cas-effector fusion protein.

15. Said conjugate of CRISPR-Cas protein-guide RNA(s) complex of claim 14, wherein said Cas protein is a recombinant engineered endonuclease comprising at least two cysteines, and at least one of the said cysteines are introduced by site directed mutations, and the said cysteines are conjugated with molecules for epitope masking and/or targeted delivery.

16. Said conjugate(s) of a CRISPR-Cas protein-guide RNA complex of claim 1, wherein said complex is PEGylated.

17. Said conjugate(s) of CRISPR-Cas protein-guide RNA(s) complex of claim 1 comprising covalently linked molecules for targeted cellular delivery.

18. Pharmaceutical agents comprising conjugates of a CRISPR-Cas protein-guide RNA complex.

19. Pharmaceutical agents comprising guide RNA conjugates and mRNA, plasmid, or viral vector encoding Cas protein to form said conjugate(s) of CRISPR-Cas protein-guide RNA complex of claim 18 in targeted cells.

20. A method of gene editing with conjugates of a CRISPR-Cas protein guide RNA complex, comprising the following steps:
a. cleaving DNA to be edited, leading to a double strand break or a nick;
b. hybridizing the resulting single DNA strand of the cleavage product with the 3′-homology arm of conjugated donor template and extending the 3′-end of complementary broken strand using said template to edit the target gene by introducing insertions, deletions or point mutations included in the gene editing sequence, wherein the conjugated donor DNA template was hypomethylated or hypermethylated at the 5-position of its cytosines.
